(19) United States
(12) Patent Application Publication
(10) Pub. No.: US 2024/0109855 A1
(43) Pub. Date: Apr. 4, 2024

(54) ANTI-QUORUM SENSING, ANTI-BIOFILM, AND INFLAMMATION ATTENUATING COMPOUNDS, COMPOSITIONS, AND METHODS OF USING SAME

(71) Applicants: B. G. NEGEV TECHNOLOGIES AND APPLICATIONS LTD., AT BEN-GURION UNIVERSITY, Beer Sheva (IL); VELLORE INSTITUTE OF TECHNOLOGY, CHENNAI, Chennai (IN)

(72) Inventors: Jacob GOPAS, Beer Sheva (IL); Ariel KUSHMARO, Beer Yahkov (IL); Yael SHLICHTER, Beer Sheva (IL); Rajendran SARAVANAKUMAR, Trichy (IN); Subramani MUTHURAMAN, Chennai (IN)

(21) Appl. No.: 18/271,948

(22) PCT Filed: Jan. 13, 2022
(86) PCT No.: PCT/IL2022/050054

§ 371 Date: Jul. 12, 2023

Related U.S. Application Data
(60) Provisional application No. 63/137,297, filed on Jan. 14, 2021.

(51) Int. Cl. C07D 295/104; A61P 31/04; C07D 211/76

(52) U.S. Cl. CPC C07D 295/104; A61P 31/04; C07D 211/76

(57) ABSTRACT

The present invention is directed to compounds having anti quorum sensing activity, anti-inflammatory activity or both, compositions comprising same, and methods of using same, such for treating a subject afflicted with a disease.

### CROSS-REFERENCE TO RELATED APPLICATIONS

This application claims the benefit of priority of U.S. Provisional Patent Application No. 63/137,297, filed Jan. 14, 2021 entitled “ANTI-QUORUM SENSING, ANTI-BIOFILM, AND INFLAMMATION ATTENUATING COMPOUNDS, COMPOSITIONS, AND METHODS OF USING SAME”, the contents of which are incorporated herein by reference in their entirety.

### FIELD OF INVENTION

The present invention relates to synthetic anti-quorum sensing, anti-biofilm and inflammation attenuating compounds, such as derivatives of Coumaperine, Piperlongumine, and Curcumin, including methods of using same.

### BACKGROUND

One way for bacteria to resist or tolerate antibacterial agents is to develop and live in biofilms, that are difficult to eliminate. The biofilm structure provides bacteria with physical stability enabling them to handle various environmental challenges, and especially to decrease permeability of antibiotic agents, and provide fertile ground for bacterial gene transfer including antibiotic resistant genes. To develop such biofilms bacteria often use a characteristic behavior termed Quorum Sensing (QS). This is a wide-spread phenomenon that occurs via chemical signaling molecules known as auto-inducers (AIs). QS provides bacteria with information regarding population density and synchronized gene expression. QS is also crucial to gene activating processes related to bacterial virulence, such as toxin secretion, host penetration, and colonization.

The persistence of biofilms on medical devices (catheters, feeding, waste tubes, implants etc.), as well as chronic infections in the host, generates a heavy burden on the health care system. Biofilms may induce persistent infections that are difficult to eradicate and cause the release of pro-inflammatory cytokines. In addition, catheters have a high risk of being contaminated by opportunist and/or obligate pathogens resulting in catheter failure, catheter-related infections, pervasiveness in hospital environments, and the dissemination of antimicrobial resistance. Such infections are characterized by inflammation and can also lead to implant failure.

Inflammation is regulated by a number of cellular factors including nuclear factor kappa B (NF-kB), a transcription factor that regulates many genes involved in inflammation, cell proliferation, and apoptosis, and whose expression is known to be up-regulated in inflammatory diseases. The NF-kB family comprises five members, RelA(p65), relB, c-Rel, p105/p50, and p100/p52. To act as transcription factors they form dimers, for example p65 and p50. Studies have shown that burn wound patients have a higher risk of infection by the opportunist bacterium Pseudomonas aeruginosa (PA) and that the QS system contributes to PA's virulence in a burn wound model. Such bacteria can also modulate the NF-kB signaling pathway, depending on the host cellular status that most benefits the pathogen.

Natural compounds from plants have long been used to treat microbial infections and have gained much attention as a source of quorum sensing inhibitors. For example, malabaricone C isolated from the bark of Myristica cinnamomea inhibited quorum sensing regulated biofilm formation in P. aeruginosa (PAO1) and violacein production by Chromobacterium violaceum (CV026). Recently, dietary phytochemicals with medical uses in humans have been investigated for anti-quorum sensing activity owing to their non-toxicity. In addition, many plant-derived natural products are known to contain NF-kB modulatory activity.

One example for a plant derived antimicrobial compound is curcumin, a major constituent of turmeric that was found to inhibit quorum sensing regulated biofilm formation in uropathogens. Piperine, a bioactive constituent of black pepper was also shown to inhibit biofilm-formation by interfering with quorum sensing activity in Streptococcus mutans. An additional natural product, coumaperine, is an amide alkaloid found in white pepper (Piper Nigrum), structurally similar to Piperine whose bioactivity has only been scarcely explored. Nonetheless, application of compounds having an antimicrobial activity may induce selection favoring resilience bacteria resistant to antibiotics.

Therefore, there is still a great need for synthetic compounds, e.g., derivatives of natural compounds as described above, having any one of: anti-QS activity, anti-biofilm activity, NF-kB signaling suppressing activity, cytotoxic activity, or any combination thereof, which also possibly lack an antimicrobial activity.

### SUMMARY

According to a first aspect, there is provided a compound represented by Formula I:

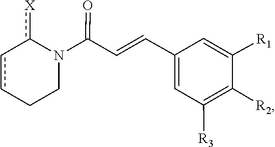

wherein: [symbol] represents a single or a double bond; X comprises H, S, O, NH or NH₂; each of R₁, R₂ and R₃ independently represents hydrogen, a substituent is selected from the group comprising halogen, nitro, —OR, —NR₂, —NHR, —SR, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; wherein each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; if R₁ and R₃ are hydrogen, then R₂ is devoid of halo, —OR, and nitro; or if R₁, R₃ or both are —OR, then R₂ is devoid of —OR; and wherein at least one of R₁, R₂ and R₃ represents the substituent.

According to another aspect, there is provided a compound represented by Formula IIA:

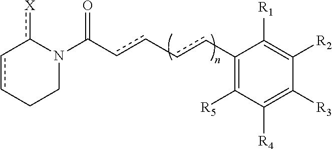

### or by Formula IIB:

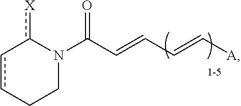

wherein: A is selected form the group consisting of optionally substituted heteroaryl, optionally substituted bicyclic aryl, optionally substituted bicyclic heteroaryl, optionally substituted bicyclic cycloalkyl, optionally substituted bicyclic heterocyclyl, optionally substituted aliphatic polycyclyl, optionally substituted aromatic polycyclyl and optionally substituted heteroaromatic polycyclyl; represents a single or a [symbol] double bond; X comprises H, S, O, NH or NH₂; n is 1 to 5; each R₁, R₂, R₃, R₄ and R₅ independently represents hydrogen or a substituent selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; wherein each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; or wherein (i) R₁ and R₂ or (ii) R₃ and R₂ are interconnected, so as to form a cyclic ring optionally comprising at least one heteroatom; and if n is 1, then at least two of R₁, R₂, R₃, R₄ and R₅ independently represent the substituent.

According to another aspect, there is provided a compound represented by Formula III:

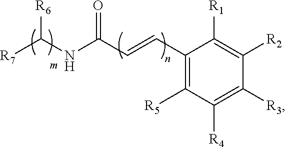

wherein each n and m is independently 1 to 5; each R₁, R₂, R₃, R₄, R₅ and R₆ independently represents hydrogen or a substituent selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; each R independently represents hydrogen, or is selected from the group comprising optionally substituted C₁-C₁₀ alkyl, optionally substituted ₃-C₁₀ cycloalkyl, optionally substituted C₃-C₁₀ heterocyclyl, optionally substituted heteroaryl, and optionally substituted aryl or a combination thereof; or wherein (i) R₁ and R₂ or (ii) R₃ and R₂ are interconnected, so as to form a cyclic ring optionally comprising at least one heteroatom; and R₇ is selected from the group comprising hydrogen, halogen, vinyl, allyl, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof.

According to another aspect, there is provided a compound represented by Formula IV:

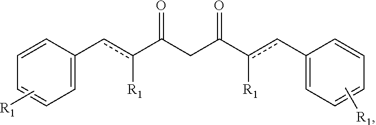

wherein: [symbol] represents a single or a double bond; each R₁ independently represents hydrogen or a substituent selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; each R independently represents hydrogen or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; and wherein the compound is devoid of

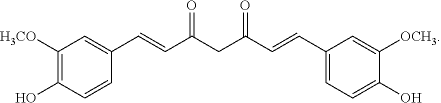 [SMILES: COc1cc(/C=C/C(=O)CC(=O)/C=C/c2ccc(O)c(OC)c2)ccc1O]

According to another aspect, there is provided a pharmaceutical composition comprising the compound of the invention and an acceptable carrier.

According to another aspect, there is provided a pharmaceutical composition for use in treatment of a subject in need of any one of: inhibition of quorum sensing, inhibition of biofilm production, inhibition of nuclear factor kappa B (NF-kB) signaling, induction of cell death, treatment of an infectious disease, treatment of an inflammatory disease, treatment of cell proliferation related disease, and any combination thereof, wherein the composition comprises a compound represented by Formula V:

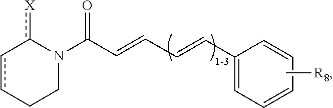

wherein: [symbol] represents a single or a double bond; R₈ represents hydrogen or a substituent selected from the group comprising halogen, nitro, —OR, —NR₂, —NHR, —SR, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof.

According to another aspect, there is provided a method for treating a subject afflicted with a disease selected from the group consisting of: an inflammatory disease, an infectious disease, an amyloid aggregates-related disease, and cell proliferation related disease, comprising: administering to the subject a therapeutically effective amount of any one of: (i) the compound of the invention; and (ii) the pharmaceutical composition of the invention, thereby treating a subject afflicted with the disease selected from the group consisting of: an inflammatory disease, an infectious disease, and an amyloid aggregates-related disease.

In some embodiments, the compound is selected from the group consisting of:

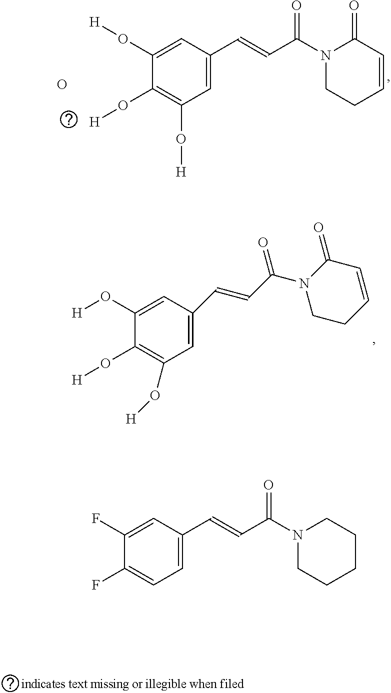 [SMILES: O ; O=C1C=CCCN1C(=O)/C=C/c1cc(O)c(O)c(O)c1 ; O=C1C=CCCN1C(=O)/C=C/c1cc(O)c(O)c(O)c1 ; O=C(/C=C/c1ccc(F)c(F)c1)N1CCCCC1]

In some embodiments, the compound is represented by Formula IIB1:

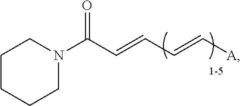

wherein A comprises an optionally substituted polycyclyl comprising between 2 and 6 aromatic rings.

In some embodiments, A is selected from the group consisting of pyrene, naphthalene, biphenyl, terphenyl, fluorene, anthracene phenanthrene, phenalene, tetracene, chrysene, and perylene or any combination thereof.

In some embodiments, R₁ and R₅ are hydrogens and any of R₂, R₃, and R₄ independently represents a substituent selected from the group comprising halogen, nitro, —OR, —SR, —NR₂, —OCOR, —CO₂R, and —CN or any combination thereof.

In some embodiments, R₇ is selected from the group comprising hydrogen, halogen, optionally substituted C₁-C₆ alkyl, optionally substituted C₁-C₄ alkenyl, C₁-C₆ alkyl-R₈, wherein R₈ is selected from the group consisting of —OCOR, —CO₂R, —NCOR, —CONR₂, —SO₂R, optionally substituted C₃-C₈ cycloalkyl, and optionally substituted aryl, or any combination thereof.

In some embodiments, the compound is represented by Formula IVA:

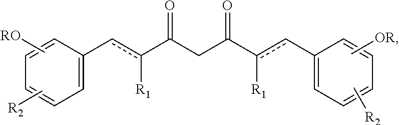

wherein [symbol] represents a single or a double bond; each R independently represents hydrogen, an optionally substituted C₁-C₁₀ alkyl, or an optionally substituted C₃-C₁₀ cycloalkyl; each R₂ independently represents a substituent selected from the group comprising —OR, —SR, —NR₂, —OCOR, —CO₂R, —NCOR, —CONR₂, —SO₂R or any combination thereof. R1 is not defined.

In some embodiments, the pharmaceutical composition is for use in treatment of a subject in need of any one of: inhibition of quorum sensing, inhibition of biofilm production, inhibition of nuclear factor kappa B (NF-kB) signaling, induction of cell death, treatment of an infectious disease, treatment of an inflammatory disease, treatment of cell proliferation related disease, and any combination thereof.

In some embodiments, the compound is selected from the group consisting of:

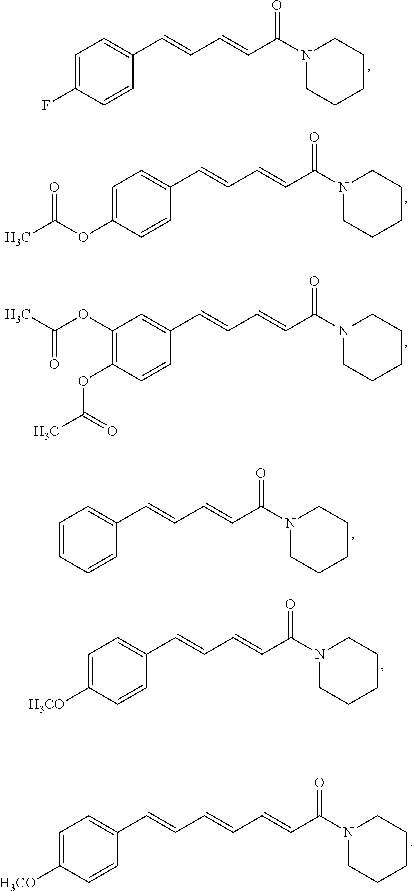

In some embodiments, any one of: the infectious disease and inflammatory disease is induced by a bacterium or a fungus.

In some embodiments, the microorganism is a bacterium or a fungus.

In some embodiments, the bacterium belongs to a genus selected form the group consisting of: Vibrio, Salmonella, Staphylococcus, Pseudomonas, Chromobacterium, Agrobacterium, Bacillus, Acinetobacter, Streptococcus, Salmonella, Envinia, and Escherichia.

In some embodiments, the bacterium is selected from the group consisting of: Chromobacterium violaceum, Agrobacterium tumefaciens, Bacillus subtilis, Staphylococcus aureus, Acinetobacter baumannii, Streptococcus sobrinus, Streptococcus mutans, Salmonella enterica, Envinia amylovera, and Escherichia coli.

In some embodiments, the cell comprises a cancerous cell.

In some embodiments, the cell proliferation related disease comprises cancer.

In some embodiments, the subject is afflicted with inflammation, an infectious disease, or both.

In some embodiments, the infectious disease comprises a load of a microorganism, a biofilm derived therefrom, or both.

In some embodiments, the pharmaceutical composition is characterized by having a median lethal concentration (LC50) of 1-500 μM.

In some embodiments, the subject is afflicted with a disease selected from the group consisting of: Chron's disease, Ulcerative colitis, an auto-immune disease, Hodgkin lymphoma, cystic fibrosis, psoriasis, pneumonia, a blood stream infection, an infectious wound, an infectious burn wound, a urinary tract infection, a medical instrument related septic or aseptic complication, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes the structures of malabaricone C, piperine (PIP), and curcumin.

FIG. 2 includes the structures of mono, di and triconjugated coumaperine derivatives.

FIG. 3 includes a procedure for the synthesis of a monoconjugated coumaperine derivative, e.g., CP-237.

FIG. 4 includes a procedure for the synthesis of monoconjugated coumaperine derivatives.

FIG. 5 includes the structures of non-limiting examples of di and triconjugated coumaperine derivatives synthesized herein.

FIG. 6 includes the structures of non-limiting examples of diconjugated coumaperine derivatives synthesized herein.

FIG. 7 includes a vertical bar graph showing a dose response of NF-kB activation according to an NF-kB reporter gene luciferase assay. L428 cells were stably transfected with the NF-kB luciferase reporter gene. The cells were incubated with the compounds at different concentrations for 2 hours. Only compounds which showed NF-kB inhibition are shown. The results represent the percentage of NF-kB activation as compared to vehicle (DMSO) treated cells. All samples were normalized to protein concentration. Mean+SD, Two-way ANOVA and Tukey's multiple comparison test. 95% confidence interval (p-values *≤0.0332, **≤0.0021, ***≤0.0002).

FIGS. 8A-8D include fluorescent micrographs and vertical bar graphs showing NF-kB inhibition in A549 cells. A549 cells were fixed in paraformaldehyde and immunostained with mouse-anti p65 and fluorescent goat anti-mouse IgG. Localization of p65 (8A) without treatment—inactive p65 in the cytoplasm. (8B) After activation with 2.5 ng/ml TNFα for 15 min, activated p65 in the nucleus. PL-18, Curcumin (CU) or DMSO were added to the cells at 160 μM for 120 min in DMSO (0.16%) then TNFα at 2.5 ng/ml was added for 15 min. (8C) Activated cells (strong nuclear green fluorescence) as a percentage of all the cells in the analyzed field. (8D) Ratio of the mean nuclear/cytoplasm fluorescence intensity values in the analyzed fields. Mean±SD of triplicate samples in two independent experiments. One-way ANOVA comparison of the treatment groups to the control group DMSO TNF. 95% confidence interval (p-values *≤0.0332, **≤0.0021, ***≤0.0002). For the comparison between the CU and the CU TNF groups and between PL-18 and PL-18 TNF in 8D—unpaired two tailed t-test with 95% confidence interval, p-value is 0.0419 (*) for CU-CU TNF and for PL-18-PL-18 TNF is 0.9323 (ns).

FIGS. 9A-9D include fluorescent micrographs showing NF-kB inhibition. A549 cells were treated with TNFα (2.5 ng/ml) and/or different positive QS CP derivatives, then fixed and immunostained with anti-p65 and anti-mouse IgG conjugate to Alexa AF488 (green) and nuclear staining with DAPI (blue). (9A) Negative control: treated with the solvent DMSO (0.16%) for 120 min; Positive control: treated with TNFα for 15 min as follows—DMSO (0.16%) for 120 min with TNFα (2.5 ng/ml) for 15 min. (9B) CP-154 and CP-158 at 160 μM for 120 min with or without TNFα (2.5 ng/ml) for 15 min. (9C) CP-286 and CP-215 at 160 μM for 120 min with or without TNFα (2.5 ng/ml) for 15 min. (9D) Curcumin (CU) was used as a positive control with the same experimental design as in 9B or 9C.

FIG. 10 includes a structure to activity relationship of methoxy (CP-38) and thiomethyl (CP-147) diconjugated coumaperine derivatives, disclosed hererin.

FIG. 11 includes the structures of non-limiting examples of derivatives of Piperlongumine (PL) synthesized herein.

FIG. 12 includes the structures of non-limiting examples of derivatives of Curcumin (CU) synthesized herein.

FIGS. 13A-13C include vertical bar graphs showing the dose response inhibition of QS by PL-18. Bioluminescence indicator bacteria containing the P. aeruginosa reporter genes rhlRI (13A) and lasRI (13B) were used. The reporter bacteria were incubated with and range of PL-18 concentrations or DMSO, together with (+AI) or without (−AI) the proper auto-inducer. Bioluminescence was recorded at 15 min intervals for 10 hrs at 37° C. The maximum signal in each group was normalized to the inducer's bioluminescence maximum signal with 0.5% DMSO. Mean±SD, One-way ANOVA followed by Dunett's multiple comparisons test was performed (p-value ***≤0.0002). N=9 (triplicate samples in three independent experiments). (13C) RT-qPCR of P. aeruginosa (PA01) QS genes. PA01 was co-incubated with two doses of PL-18 for seven hours, lysed and total RNA was extracted and converted to cDNA followed by target genes qPCR amplification. N≥16, Mean±SD. Two-way ANOVA, 95% confidence interval.

FIGS. 14A-14B include vertical bar graphs showing that PL-18 inhibition effects on secretion of virulence factors by P. aeruginosa. (14A) Pyocyanin was extracted in the chloroform-HCl method and measured in a plate-reader at 380 nm. (14B) Determination of rhamnolipids content was measured at 570 nm of the cell-free supernatant after adjustment to pH-2. All results were normalized and compere to the control group-DMSO 0.16%. Mean±SD, One-way ANOVA followed by Dunett's multiple comparisons test (p-values *≤0.0332, ***≤0.0002). N=9 (in three independent experiments).

FIGS. 15A-15B include fluorescent micrographs and a vertical bar graph showing that Pl-18 inhibits P. aeruginosa biofilm formation. The biofilm was grown under continuous flow conditions for 72 hr with 10 μM, 40 μM PL-18 or 0.04% DMSO. CLSM images visualize viable bacteria stained green and dead bacteria red using the BacLight® Dead/Live Kit. Scanned areas were 318 μm×318 μm. (15A) IMARIS images. Scale bar is 50 μm. (15B) A graph showing the mean±SD of the biofilm volume generated from three independent sets of flow-cell experiments. Two-way ANOVA Bonferroni's multiple comparisons test, 95% confidence interval (p-values ***≤0.0002).

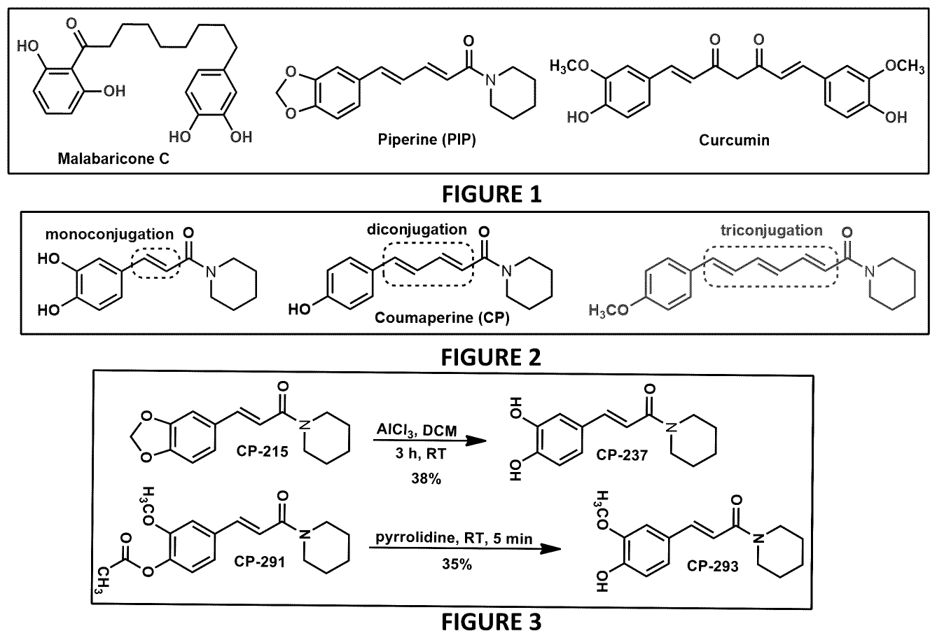

### DETAILED DESCRIPTION

The present invention provides compounds, compositions comprising same, and methods of using same, such, but not limited to, inhibiting quorum sensing.

### Compound

In one aspect, there is a compound represented by a Formula I:

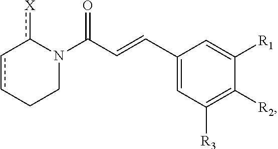

wherein: [symbol] represents a single or a double bond; X comprises H, S, O, NH or NH₂; each of R₁, R₂ and R₃ independently represents hydrogen, a substituent is selected from the group comprising halogen, nitro, —OR, —NR₂, —NHR, —SR, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; wherein each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; if R₁ and R₃ are hydrogen, then R₂ is devoid of halo, —OR, and nitro; if R₁, R₃ or both are —OR, then R₂ is devoid of —OR; or wherein R₁ and R₂ are interconnected, so as to form a cyclic ring optionally comprising at least one heteroatom (e.g. 3-7, 4, 5, 6-membered aromatic, heteroaromatic ring and/or an aliphatic ring optionally comprising a heteroatom).

In some embodiments, at least one of R₁, R₂ and R₃ represents the substituent, wherein the substituent is as described herein.

In some embodiments, the compound is represented by Formula IA:

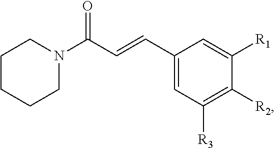

wherein R₁, R₂ and R₃ are as described hereinabove. In some embodiments, at least one of R₁, R₂ and R₃ is a substituent comprising any one of —OCOR, —CO₂R, —NCOR, —CONR₂, or a combination thereof, and wherein each R independently represents hydrogen, or is selected from the group comprising optionally substituted C₁-C₁₀ alkyl. In some embodiments, at least one of R₁, R₂ and R₃ is a substituent comprising halo, C₁-C₆ haloalkyl, —OCOR or any combination thereof.

In some embodiments, the compound is represented by Formula IA and at least two of R₁, R₂ and R₃ independently represent a substituent selected from the group comprising halogen, nitro, —OR, —NR₂, —NHR, —SR, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; and wherein R is as described hereinabove.

In some embodiments, the compound of the invention is selected from the group consisting of:

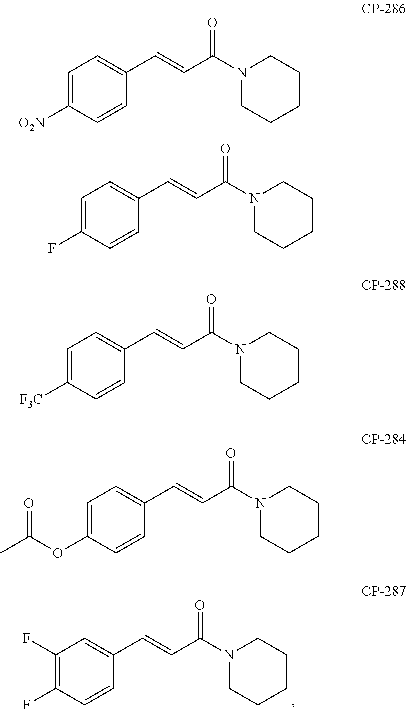

or any combination thereof.

In some embodiments, the compound is represented by Formula IB:

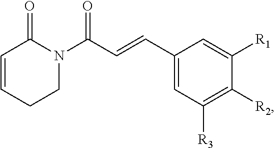

wherein each of R₁, R₂ and R₃ independently represent hydrogen or a substituent selected from the group comprising —OR, —NR₂, —NHR, —SR, —OCOR, —CO₂R, —NCOR, —CONR₂, wherein at least two of R₁, R₂ and R₃ represent the substituent; and wherein R is as described hereinabove.

In some embodiments, the compound of the invention is selected from the group consisting of:

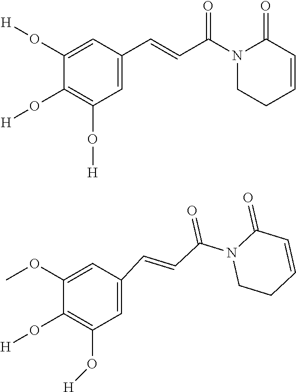 [SMILES: O=C1C=CCCN1C(=O)/C=C/c1cc(O)c(O)c(O)c1 ; COc1cc(/C=C/C(=O)N2CCC=CC2=O)cc(O)c1O]

In some embodiments, the compound of the invention is or comprises any one of:

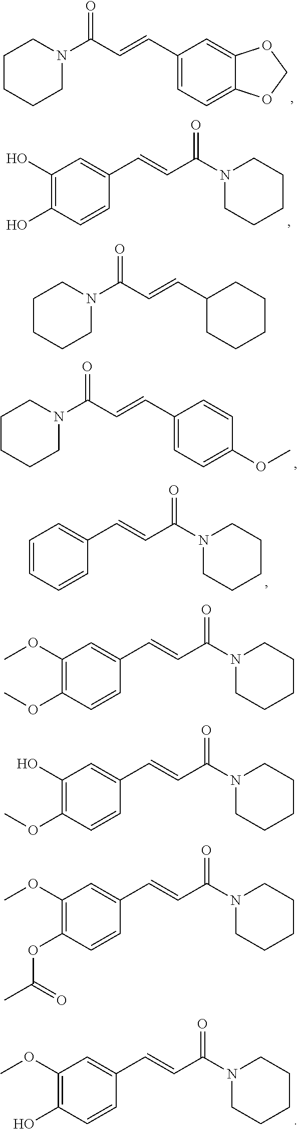

In some embodiments, the compound of the invention is represented by Formula IC:

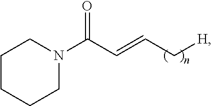

wherein n is between 1 and 10.

In some embodiments, the compound of the invention is represented by Formula IC, wherein n is 1, 2, 3, 4, 5, or 6.

In some embodiments, the compound of the invention represented by Formula IB is devoid of:

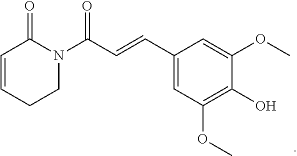 [SMILES: COc1cc(/C=C/C(=O)N2CCC=CC2=O)cc(OC)c1O]

In some embodiments, the compound of the invention is represented by Formula IIA:

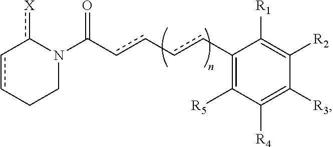

### or by Formula IIB:

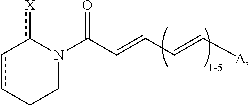

wherein: A is selected form the group consisting of optionally substituted heteroaryl, optionally substituted bicyclic aryl, optionally substituted bicyclic heteroaryl, optionally substituted bicyclic cycloalkyl, optionally substituted bicyclic heterocyclyl, optionally substituted aliphatic polycyclyl, optionally substituted aromatic polycyclyl and optionally substituted heteroaromatic polycyclyl; represents a single or a double bond; X comprises H, S, O, NH or NH₂; n is 1 to 5; each R₁, R₂, R₃, R₄ and R₅ independently represents hydrogen or a substituent selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; wherein each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; or wherein (i) R₁ and R₂ or (ii) R₃ and R₂ are interconnected, so as to form a cyclic ring optionally comprising at least one heteroatom (e.g. 3-7, 4, 5, 6-membered aromatic, heteroaromatic ring and/or an aliphatic ring optionally comprising a heteroatom); and if n is 1, then at least two of R₁, R₂, R₃, R₄ and R₅ independently represent the substituent.

In some embodiments, the compound is represented by Formula IIB1:

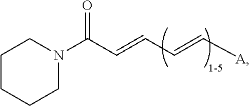

wherein A comprises an optionally substituted polycyclyl comprising between 2 and 6 aromatic rings. In some embodiments, the compound is represented by Formula IIB1, wherein A is selected from the group consisting of pyrene, naphthalene, biphenyl, terphenyl, fluorene, anthracene phenanthrene, phenalene, tetracene, chrysene, and perylene or any combination thereof.

In some embodiments, the compound comprises any one of:

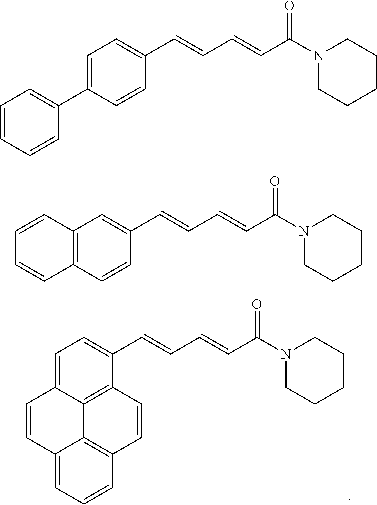 [SMILES: O=C(/C=C/C=C/c1ccc(-c2ccccc2)cc1)N1CCCCC1 ; O=C(/C=C/C=C/c1ccc2ccccc2c1)N1CCCCC1 ; O=C(/C=C/C=C/c1ccc2ccc3cccc4ccc1c2c34)N1CCCCC1]

In some embodiments, the compound is represented by Formula IIA1:

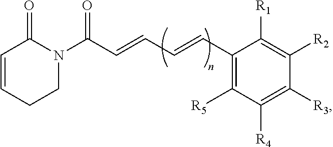

wherein n, R₁, R₂, R₃, R₄ and R₅ are as described herein.

In some embodiments, the compound is represented by Formula IIA2:

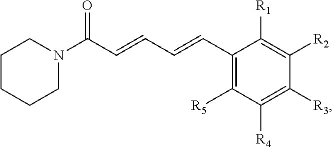

wherein R₃ represents any of —OR, —SR, —NR₂, —CO₂R, —NCOR, —CONR₂; and at least one of R₁, R₂, R₅ and R₄ is selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof, and wherein R is as described herein. In some embodiments, R₃ represents any of —OH, —SR, —NR₂, and at least one of R₁, R₂, R₅ and R₄ is selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof, and wherein R is as described herein.

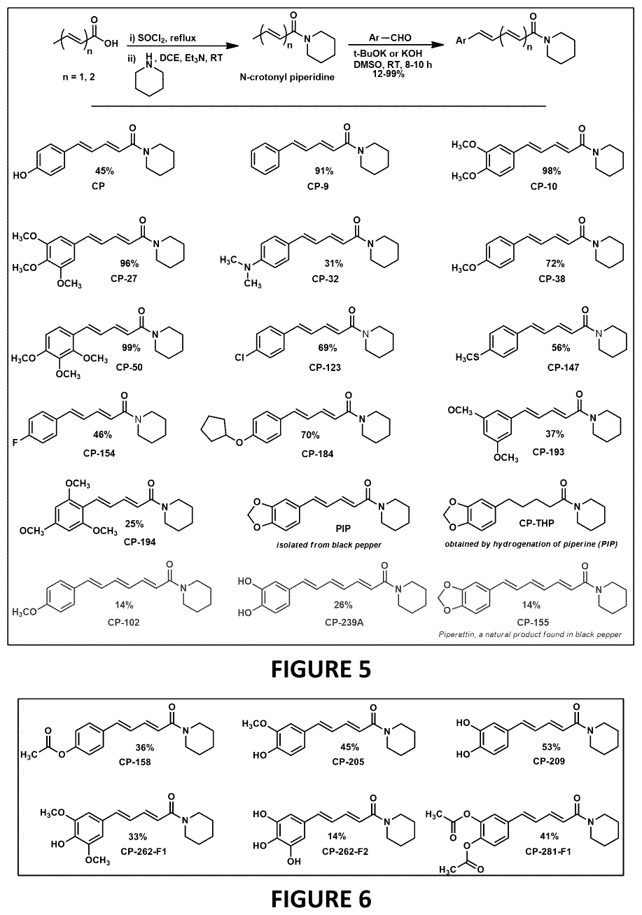

In some embodiments, the compound of the invention comprises any one of the compounds represented in any one of the FIGS. 5 and 6, or a combination thereof.

In some embodiments, the compound represented by Formula IIA2 is devoid of any one of:

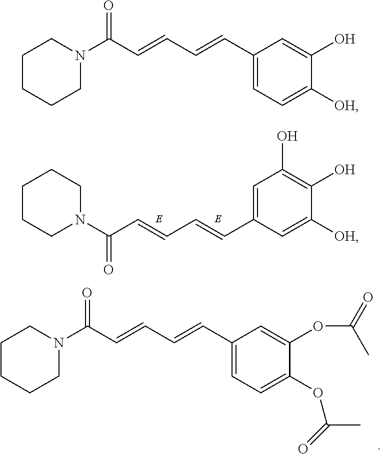

In some embodiments, the compound is represented by Formula IIA2, wherein at least one of R₁, R₂, R₃, R₅ and R₄ represents a substituent selected from —OR₁, —SR, —N(R)₂, —OCOR₁, or —CO₂R₁, wherein R1 represents an optionally substituted C₃-C₁₀ cycloalkyl, optionally substituted C₃-C₁₀ heterocyclyl, optionally substituted heteroaryl, and optionally substituted aryl or a combination thereof; and wherein each of R₁, R₂, R₃, R₅ and R₄ independently represents hydrogen or is selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof, and wherein R is as described herein.

In some embodiments, the compound of the invention is or comprises any of

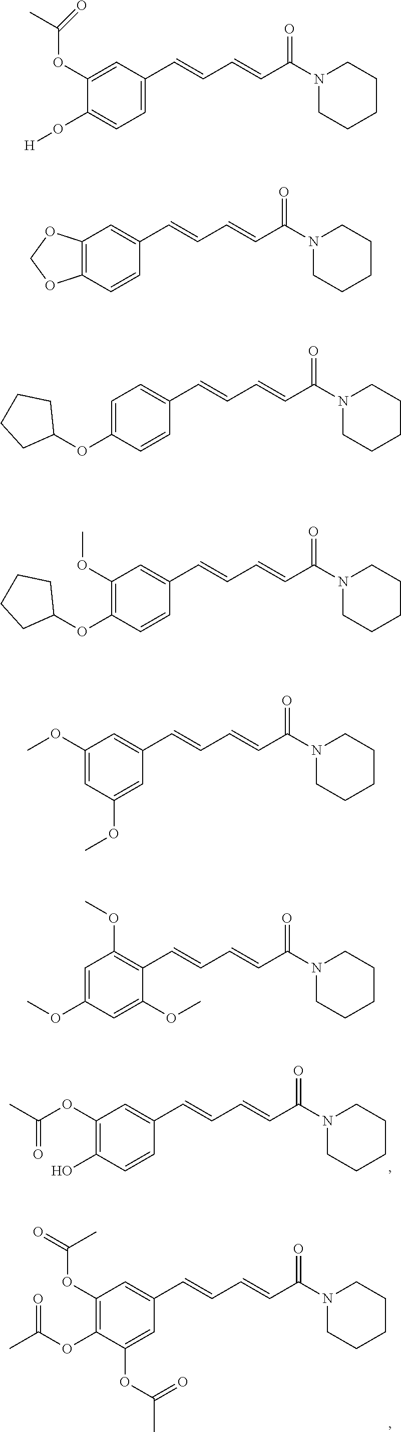

or any combination thereof.

In some embodiments, the compound of the invention is represented by Formula III:

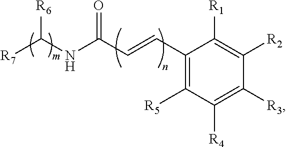

wherein each n and m is independently 1 to 5; each R₁, R₂, R₃, R₄, R₅ and R₆ independently represents hydrogen or a substituent selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; or wherein (i) R₁ and R₂ or (ii) R₃ and R₂ are interconnected, so as to form a cyclic ring optionally comprising at least one heteroatom (e.g. 3-7, 4, 5, 6-membered aromatic, heteroaromatic ring and/or an aliphatic ring optionally comprising a heteroatom); and R₇ is selected from the group comprising hydrogen, halogen, vinyl, allyl, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof.

In some embodiments, R₁ and R₅ are hydrogens and any of R₂, R₃, and R₄ independently represents a substituent selected from the group comprising halogen, nitro, —OR, —SR, —NR₂, —OCOR, —CO₂R, and —CN or any combination thereof.

In some embodiments, wherein R₇ is selected from the group comprising hydrogen, halogen, optionally substituted C₁-C₆ alkyl, optionally substituted C₁-C₄ alkenyl, C₁-C₆ alkyl-R₈, wherein R₈ is selected from the group consisting of —OCOR, —CO₂R, —NCOR, —CONR₂, —SO₂R, optionally substituted C₃-C₈ cycloalkyl, and optionally substituted aryl, or any combination thereof.

In some embodiments, the compound of the invention is or comprises any of:

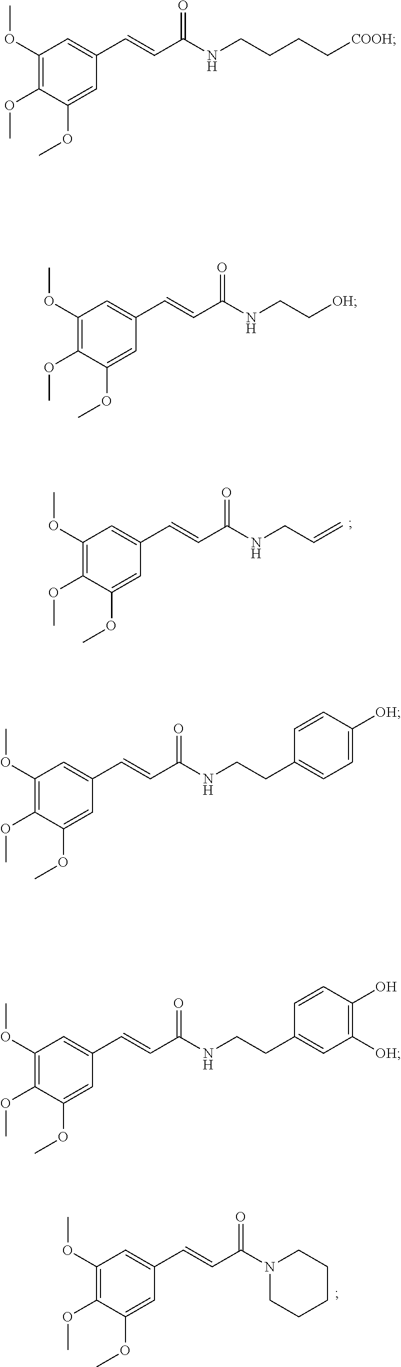 [SMILES: COc1cc(/C=C/C(=O)NCCCCC(=O)O)cc(OC)c1OC ; COc1cc(/C=C/C(=O)NCCO)cc(OC)c1OC ; C=CCNC(=O)/C=C/c1cc(OC)c(OC)c(OC)c1 ; COc1cc(/C=C/C(=O)NCCc2ccc(O)cc2)cc(OC)c1OC ; COc1cc(/C=C/C(=O)NCCc2ccc(O)c(O)c2)cc(OC)c1OC ; COc1cc(/C=C/C(=O)N2CCCCC2)cc(OC)c1OC]

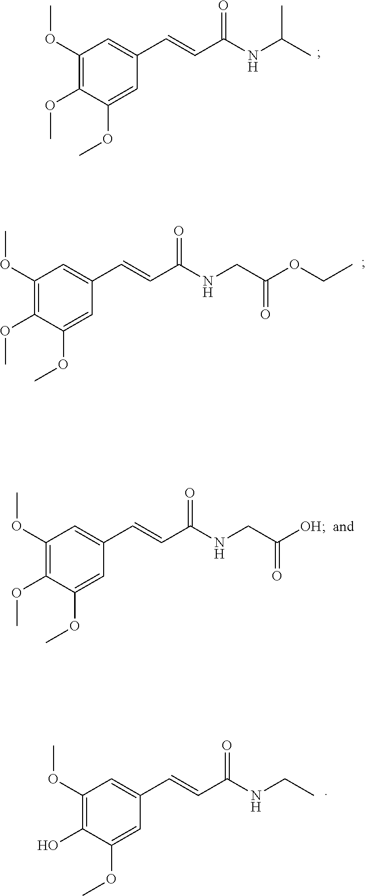

In some embodiments, the compound of the invention is or comprises any of:

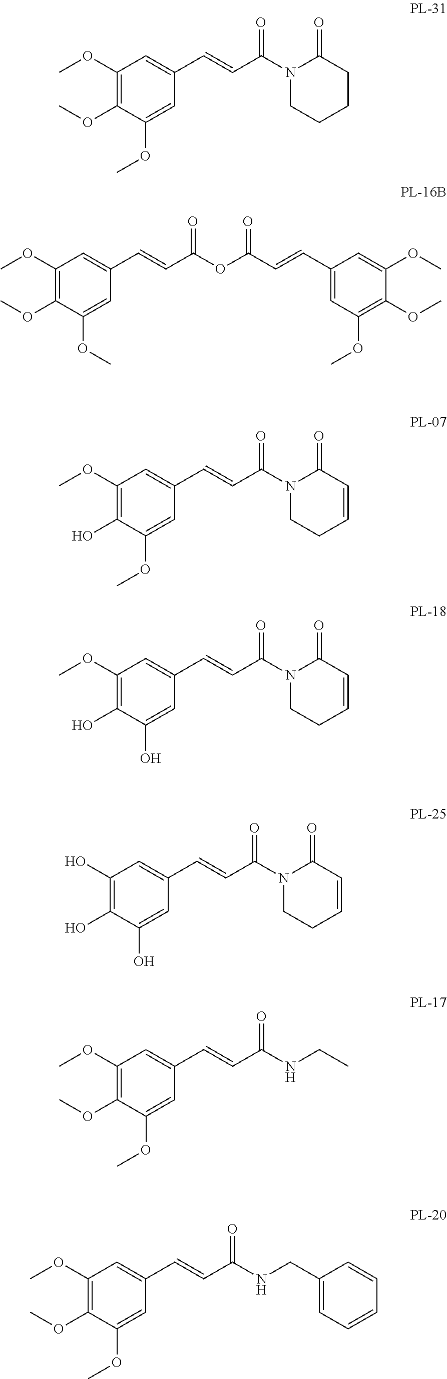

and any combination thereof.

In some embodiments, the compound of the invention is represented by Formula IV:

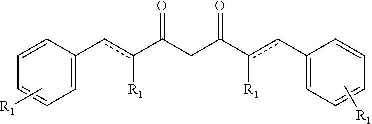

wherein: [symbol]
represents a single or a double bond; each R₁ independently represents hydrogen or a substituent selected from the group comprising halogen, nitro, —OR, —SR, —NR₂, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; each R independently represents hydrogen or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof, and wherein the compound is devoid of

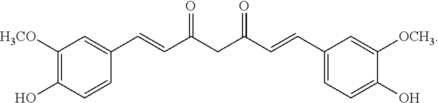 [SMILES: COc1cc(/C=C/C(=O)CC(=O)/C=C/c2ccc(O)c(OC)c2)ccc1O]

In some embodiments, each R₁ independently represents one or more substituents (e.g., 2, 3, 4, or 5).

In some embodiments, the compound of the invention is represented by Formula IVA:

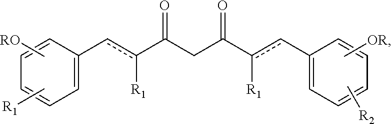

wherein represents a single or a double bond; R₁ and R are as described hereinabove; each R₂ independently represents a substituent selected from the group comprising —OR, —SR, —NR₂, —OCOR, —CO₂R, —NCOR, —CONR₂, —SO₂R or any combination thereof, and if is a double bond, then at least one R₂ is not hydroxy.

In some embodiments, each R independently represents hydrogen, an optionally substituted C₁-C₁₀ alkyl, or an optionally substituted C₃-C₁₀ cycloalkyl.

[symbol]

In some embodiments, the compound of the invention is represented by Formula IVB:

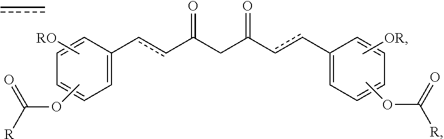

### or by Formula IVC:

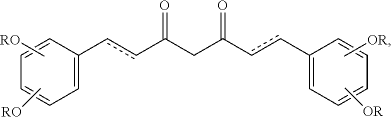

wherein R is as described hereinabove.

In some embodiments, each R independently represents hydrogen, or an optionally substituted C₁-C₁₀ alkyl. In some embodiments, each R independently represents hydrogen, or an optionally substituted C₁-C₆ alkyl.

In some embodiments, the compound of the invention is or comprises any of:

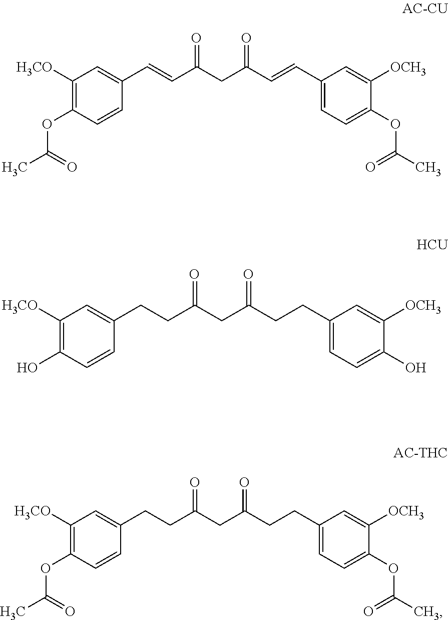

or any combination thereof.

As used herein, the term “7-10 ring” is referred to a cyclic aliphatic or aromatic compound comprising between 7 and 10 carbon atoms. In some embodiments, 7-10 ring bicyclic ring comprises between 7 and 8, between 8 and 9, between 9 and 10 carbon atoms including any value therebetween.

As used herein the term “C1-C6 alkyl” including any C1-C6 alkyl related compounds, is referred to any linear or branched alkyl chain comprising between 1 and 6, between 1 and 2, between 2 and 3, between 3 and 4, between 4 and 5, between 5 and 6, carbon atoms, including any range therebetween. In some embodiments, C1-C6 alkyl comprises any of methyl, ethyl, propyl, butyl, pentyl, iso-pentyl, hexyl, and tert-butyl or any combination thereof. In some embodiments, C1-C6 alkyl as described herein further comprises an unsaturated bond, wherein the unsaturated bond is located at 1st, 2nd, 3rd, 4th, 5th, or 6th position of the C1-C6 alkyl.

As used herein the term “(C3-C8) cycloalkyl” is referred to an optionally substituted C3, C4, C5, C6, C7, or C8 ring optionally comprises at least one unsaturated bond. In some embodiments, (C3-C10) ring comprises optionally substituted cyclopropane, cyclobutene, cyclopentane, cyclohexane, or cycloheptane.

As used herein the term “(C3-C8) heterocyclyl” is referred to an optionally substituted C3, C4, C5, C6, C7, C8 ring comprising at least one heteroatom selected from O, N, and S.

As used herein the term “(C6-C10) ring” is referred to an optionally substituted C6, C7, C8, C9 , or C10 ring. In some embodiments, (C6-C10) ring is referred to a bicyclic ring (e.g. fused ring, spirocyclic ring, biaryl ring).

As used herein the term “bicyclic heteroaryl” referred to (C6-C12) a bicyclic heteroaryl ring, wherein bicyclic (C6-C10) ring is as described herein.

As used herein the term “bicyclic aryl” referred to (C6-C12) a bicyclic aryl ring, wherein bicyclic (C6-C12) ring is as described herein.

As used herein the term “bicyclic heterocyclyl” is referred to (C6-C12) a bicyclic heterocyclic ring, wherein (bicyclic C6-C12) ring is as described herein.

As used herein the term “bicyclic cycloalkyl” is referred to (C6-C12) a bicyclic cycloalkyl ring, wherein bicyclic (C6-C12) ring is as described herein.

In some embodiments, the compound of the invention substantially comprises a single enantiomer of any one of the compounds described herein, wherein substantially is at least 70%, at least 75%, at least 80%, at least 85%, at least 90%, at least 92%, at least 93%, at least 95%, at least 97%, at least 98%, at least 99% by weight, including any value therebetween.

In some embodiments, the composition of the invention comprises the compound of the invention, a mixture (e.g., racemic mixture) of enantiomers, or is enriched with an enantiomer of interest.

In some embodiments, the compound of the invention comprises any one of the compounds disclosed herein, including any enantiomers thereof. In some embodiments, the compound of the invention comprises a mixture of enantiomers (e.g., a racemic mixture). In some embodiments, the compound of the invention comprises any one of the compounds disclosed herein, including any salt thereof. In some embodiments, the salt of the compound is a pharmaceutically acceptable salt.

### Compositions

According to some embodiments, there is provided a composition comprising the compound of the invention. In some embodiments, the composition comprises the compound of the invention, a pharmaceutically acceptable salt thereof or both.

In some embodiments, the composition further comprises an acceptable carrier. In some embodiments, the carrier is a pharmaceutical carrier. In some embodiments, the carrier is configured to adhere or enable the adherence of the compound of the invention to a surface.

In some embodiments, the surface is a surface of an article.

In some embodiments, the surface is a surface of a medical instrument. In some embodiments, the surface is a surface of a compartment or a vessel suitable for or configured to containing food.

In some embodiments, a medical instrument includes a catheter or a tubing. In some embodiments, a compound of the invention or a composition comprising same is used for coating a medical instrument or a device made or covered with plastic or silicon.

In some embodiments, a compound of the invention or a composition comprising same is used for reducing or inhibiting biofilm production in an oral cavity of a subject in need thereof. In some embodiments, a compound of the invention or a composition comprising same is used for wound healing. In some embodiments, the compound of the invention or a composition comprising same is integrated in a wound dressing.

In some embodiments, there is provided a method for increasing the shelf life of a food product, comprising contacting the food product with an effective amount of the compound of the invention or a composition comprising same. In some embodiments, the contacting is directly or indirectly. In some embodiments, directly contacting comprises dipping, drenching, covering, or any equivalent thereof the food product with the compound of the invention or a composition comprising same. In some embodiments, indirectly contacting comprises storing or preserving the food product in a compartment, container, or a vessel comprising the compound of the invention or a composition comprising same covering, lining, or embedded therein. In some embodiments, increasing the shelf life comprises inhibiting a pathogen. In some embodiments, a pathogen is a plant/phyto pathogen.

In some embodiments, the composition is for use in any one of: inhibition of quorum sensing, inhibition of biofilm production, inhibition of nuclear factor kappa B (NF-kB) signaling, induction of cell death, treatment of an infectious disease, treatment of an inflammatory disease, and treatment of cell proliferation related disease, in a subject in need thereof.

In some embodiments, the composition is for use in any one of inhibition of quorum sensing, inhibition of biofilm production, inhibition of nuclear factor kappa B (NF-kB) signaling, induction of cell death, treatment of an infectious disease, treatment of an inflammatory disease, and treatment of cell proliferation related disease, in a subject in need thereof, wherein the composition comprises a compound represented by Formula V:

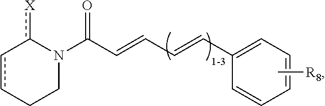

wherein R₈ represents hydrogen or a substituent selected from the group comprising halogen, nitro, —OR, —NR₂, —NR₂, —SR, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof, wherein represents a [symbol] single or a double bond; and wherein R is as described hereinabove.

In some embodiments, the composition comprises a compound represented by Formula VA:

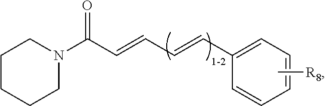

wherein R₈ is as described herein. In some embodiments, R₈ represents a single substituent, wherein the substituent is as described herein. In some embodiments, R₈ represents at least 2, at least 3, at least 4, or at least 5 substituents, wherein the substituent is as described herein.

In some embodiments, the composition comprises a compound selected from the group consisting of:

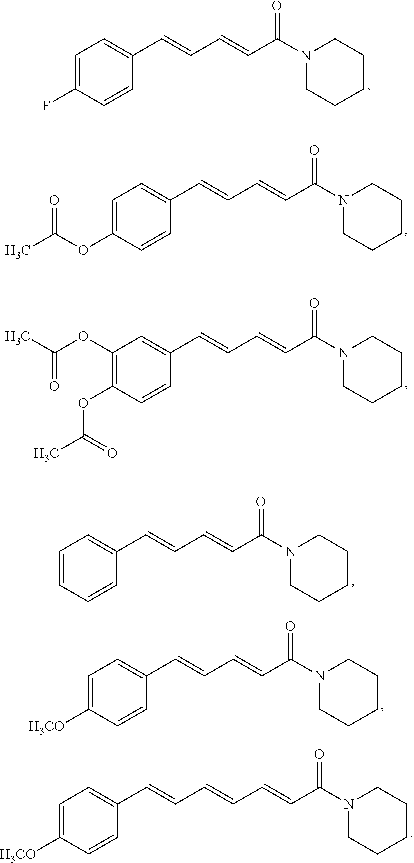

In some embodiments, the composition of the invention is for use in inhibition of quorum sensing in or on a surface. In some embodiments, the composition of the invention is for use in inhibition of biofilm production in or on a surface.

According to some embodiments, there is provided a pharmaceutical composition comprising a compound as disclosed herein, and an acceptable carrier.

In some embodiments, the pharmaceutical composition is for use in treatment of a subject in need of any one of: inhibition of quorum sensing, inhibition of biofilm production, inhibition of nuclear factor kappa B (NF-kB) signaling, induction of cell death, treatment of an infectious disease, treatment of an inflammatory disease, treatment of cell proliferation related disease, or any combination thereof.

In some embodiments, the subject is afflicted with inflammation, an infectious disease, or both.

In some embodiments, the subject is afflicted with a disease selected from: Chron's disease, Ulcerative colitis, an auto-immune disease, Hodgkin lymphoma, cystic fibrosis, psoriasis, pneumonia, a blood stream infection, an infectious wound, an infectious burn wound, a urinary tract infection, a medical instrument related septic or aseptic complication, or any combination thereof.

As used herein, the term “carrier”, “excipient”, or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods

According to some embodiments, there is provided a method for treating a disease in a subject in need thereof, the method comprising: administering to the subject a therapeutically effective amount of a pharmaceutical composition comprising the compound of the invention.

In some embodiments, the disease is selected from: an inflammatory disease, an infectious disease, an amyloid aggregates-related disease, or a cell proliferation related disease.

In some embodiments, the method comprises inducing cell death. In some embodiments, inducing cell death comprises having a cytotoxic activity. In some embodiments, a cytotoxic activity comprises a specific cytotoxic activity. In some embodiments, specific cytotoxic activity comprises a programed cell death signaling or cascade. In some embodiments, a specific cytotoxic activity is not random cytotoxic activity. In some embodiments, a specific cytotoxic activity comprises apoptosis.

In some embodiments, the method comprises inhibiting NF-kB signaling.

As used herein, the term “NF-kB signaling” encompasses any process initiated by, propagated by, or resulting from the assembly of the NF-kB transcription complex (e.g., comprising RelA, and p50) and activation of the transcription of downstream stress response and/or damage-response genes, or any equivalent and/or combination thereof.

A method for reducing or inhibiting an organic-based contamination in, on, or within a surface, the method comprising contacting a surface comprising the organic-based contamination with an effective amount of a composition comprising the compound of the invention, thereby inhibiting an organic-based contamination in, on, or within the surface.

In some embodiments, the infectious disease comprises a load of a microorganism, a biofilm derived therefrom, or both.

In some embodiments, any one of the: infectious disease and inflammatory disease is induced by a bacterium or a fungus.

In some embodiments, the microorganism is a bacterium or a fungus.

In some embodiments, the bacterium is a Gram-positive bacterium or a Gram-negative bacterium. In some embodiments, the bacterium belongs to a genus selected form: Vibrio, Salmonella, Staphylococcus, Pseudomonas, Chromobacterium Agrobacterium, Bacillus, Acinetobacter, Streptococcus, Salmonella, Erwinia, and Escherichia.

In some embodiments, the bacterium is selected from: Chromobacterium violaceum, Agrobacterium tumefaciens, Bacillus subtilis, Staphylococcus aureus, Acinetobacter baumannii, Streptococcus sobrinus, Streptococcus mutans, Salmonella enterica, Erwinia amylovera, and Escherichia coli.

In some embodiments, a cell comprises a cancerous cell.

In some embodiments, a cell proliferation related disease comprises cancer.

In some embodiments, the pharmaceutical composition is characterized by having a median lethal concentration (LC50) of 1-5 μM, 5-50 μM, 10-250 μM, 1-75 μM, 1-5 μM, 10-300 μM, 50-450 μM, 15-375 μM, 100-500 μM, 50-485 μM, or 20-95 μM. Each possibility represents a separate embodiment of the invention.

In some embodiments, the pharmaceutical composition is characterized by having a median lethal concentration (LC50) of 1-500 μM.

In some embodiments, the subject is afflicted with at least one disease selected from: an inflammatory disease, an infectious disease, an amyloid-related disease, and a cell proliferation related disease.

In some embodiments, an organic-based contamination comprises amyloid aggregation, e.g., amyloid beta.

As used herein, the term “organic” refers to any one of: a compound comprising carbon, a matter produced or derived from an organism, an organ, and an organism.

In some embodiment, contacting comprises administering. In some embodiments, contacting comprises incorporating. In some embodiment, contacting comprises administering to a subject. In some embodiments, contacting comprises incorporating to a surface. In some embodiments, contacting comprises contacting a cell. In some embodiments, a cell comprises a unicellular microorganism. In some embodiments, a cell comprises a cell of a subject. In some embodiments, a cell comprises a cell of the nerve system, a cancer cell, or both. In some embodiments, a cell of the nerve system comprises a neuron cell.

In some embodiments, the organic-based contamination is on or within a surface, an article, a cell, or a subject. In some embodiments, the subject comprises the cell.

In some embodiments, there is provided a method for treating a biofilm-related infectious disease or a symptom thereof in a subject in need thereof, the method comprising administering to a subject a therapeutically effective amount of pharmaceutical composition comprising at least one compound disclosed herein and at least one pharmaceutically acceptable carrier.

As used herein, “organic-based contaminant-related infectious disease” refers to any disease or disorder developed by a subject in response to an increased load of a microorganism, and/or a formation of a biofilm or biofouling. In some embodiments, an organic-based contaminant-related infectious disease inducing organism is selected from: bacteria, viruses, fungi, or parasites.

Non-limiting examples for symptoms of an infectious disease include, but are not limited to, fever, diarrhea, fatigue, muscle aches and coughing.

Non-limiting examples of infectious diseases include, but are not limited to, urinary tract infection, gastrointestinal infection, enteritis, salmonellosis, diarrhea, nontuberculous mycobacterial infections, legionnaires' disease, hospital-acquired pneumonia, skin infection, cholera, septic shock, periodontitis, infection, inflammatory bowel disease, ulcerative colitis (UC), Crohn's disease, and sinusitis. In some embodiments, the infection induces a condition selected from the group consisting of: bacteremia, skin infections, neonatal infections, pneumonia, endocarditis, osteomyelitis, toxic shock syndrome, scalded skin syndrome, and food poisoning.

The term “subject” as used herein refers to an animal, more particularly to non-human mammals and human organism. Non-human animal subjects may also include prenatal forms of animals, such as, e.g., embryos or fetuses. Non—limiting examples of non-human animals include: horse, cow, camel, goat, sheep, dog, cat, non-human primate, mouse, rat, rabbit, hamster, guinea pig, and pig. In one embodiment, the subject is a human. Human subjects may also include fetuses.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

In some embodiments, a composition as disclosed herein is directed to inhibiting quorum sensing and/or biofilm production by microorganisms in a living tissue or on or in an article. In some embodiments, a composition as disclosed herein is directed to inhibiting NF-kB signaling in a cell of a subject.

As used herein” the term “quorum sensing (QS)” refers to systems which modify gene expression pathways in response to dynamics of cell-population density. In some embodiments, molecules of the present invention or any derivative thereof, are used in a method for inhibiting QS. Any method known in the art can be used for evaluating the effect of a molecule on QS. A non-limiting example for QS examination comprises the use of a bioluminescence assay based on engineered bacteria lacking an active autoinducer gene and further cloned with a DNA vector comprising a fluorescent reporter gene.

As used herein, the term “biofilm” refers to a group of microorganisms adhering to one another, which are embedded within self-produced and self-secreted extracellular polymer comprising DNA, proteins, and polysaccharides. In some embodiments, a biofilm adheres to a surface on a living host. In one embodiment, a biofilm adheres to a non-living surface. In some embodiments, QS activity correlates with level of biofilm formation.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Materials

All the commercially obtained reagents/solvents for the synthesis of coumaperine and its derivatives were purchased from Spectrochem®, SRL®, Alfa Aesar®, RANKEM®, Fisher Scientific®, and used as received without further purification. Unless stated otherwise, the reactions were conducted in oven-dried glassware and under normal atmospheric conditions.

### Instrumentation

¹H NMR and ¹³C NMR spectra were recorded on Bruker 500 MHz spectrometer operating with the ¹³C resonance frequency of 125 MHz and the proton resonance frequency of 500 MHz or Bruker 400 MHz spectrometer operating with the ¹³C resonance frequency of 100 MHz and the proton resonance frequency of 400 MHz. DMSO-d6 or CDCl₃ with TMS as an internal standard was used as an NMR solvent. Data from the ¹H NMR spectroscopy are reported as chemical shift (δ ppm) with the corresponding integration values. Coupling constants (J) are reported in Hertz (Hz). Standard abbreviations indicating multiplicity were used as follows: s (singlet), br (broad), d (doublet), t (triplet), q (quartet) and m (multiplet). Data from ¹³C NMR spectra are reported in terms of chemical shift (δ ppm). IR spectra were recorded in Thermo Scientific Nicolet Nexus 470 FT-IR spectrometer and band positions are reported in reciprocal centimeters. Samples were made as pellet with KBr and recorded. High-resolution mass spectra were recorded on Electrospray Ionization mode on Agilent 6520 (Q-TOF) mass spectrometer in positive (ESI+) ion mode. Mass spectra were recorded on Perkin Elmer Clams 600/Shimadzu QP2020 GC-MS spectrometer in EI mode. Melting points were recorded with REMI DDMS 2545. The instrument is calibrated with benzoic acid before the measurement.

### Synthesis Methods

The mono, di and triconjugated coumaperine derivatives were synthesized as described. Briefly, coumaperine derivatives with one alkene bond between the aromatic ring and piperidine are termed monoconjugated coumaperine derivatives and those with two and three alkene bonds are termed di and triconjugated coumaperine derivatives, respectively (FIG. 2).

The monoconjugated coumaperine derivatives were synthesized in two steps, as described (Muthuraman et al., 2019, and Nandakumar et al., 2017). Knoevenagel condensation of aldehyde and malonic acid in presence of base yields cinnamic acid derivatives. It was then converted to the corresponding acid chloride and subsequently reacted with piperidine allowing the inventors to obtain monoconjugated coumaperine derivatives. CP-215 was demethylated to obtain dihydroxy derivative, CP-237. Demethylation was achieved with AlCl₃ at room temperature (FIG. 3).

Di and triconjugated derivatives were prepared from the corresponding aldehyde and crotonic/sorbic acid according to the synthetic procedure reported earlier (Muthuraman et al., 2019, and Nandakumar et al., 2017). Crotonic/sorbic acid was converted to acid chloride by reacting with thionyl chloride and subsequently reacted with piperidine to obtain crotonyl/sorbyl piperidine. It was then condensed with aromatic aldehydes in presence of base (KOH/t-BuOK) at room temperature to obtain the final product, di/triconjugated coumaperine derivatives (FIG. 5).

Piperlongumine was isolated according to the literature reported procedure. PL was demethylated to obtain hydroxy piperlongumines e.g., mono, di, or trihydroxy piperlongumines e.g., PL-07, PL-18 and PL-25, respectively. PL was demethylated following a modified literature reported procedure. PL was reacted with 10 eq. of AlCl₃ in dichloromethane and stirred at room temperature for 1 h. PL-07 was formed as a major product (82% yield) along with a trace amount of PL-18. The p-methoxy group was selectively demethylated. When PL was reacted with 20 equivalents of AlCl₃, formation of all three hydroxy piperlongumines was observed with PL-07 and PL-25 as minor product and PL-18 as a major product. When 30 equivalents of AlCl₃ was used, PL-18 and PL-25 were formed as major (54%) and minor (32%) products, respectively. Further increasing the AlCl₃ equivalence (50 eq.) to obtain PL-25 as major product was not fruitful. PL-31, a C2-C3 olefin saturated Piperlongumine was synthesized according to literature reported procedure. During the synthesis of PL-31, PL-16B and PL-23B were obtained as by-products. PL-16B is anhydride of 3,4,5-trimethoxy cinnamic acid and PL-23B is 2-piperidone ring opened product of PL-31. PL-17, PL-20, AE-02, 04, 10, 11, 17, 45, 68, 73 and 77 were synthesized by transamidation of corresponding amines with PL-31.

Curcumin for the present study was commercially obtained. Curcumin was acetylated using acetic anhydride in the presence of a base so as to obtain a diacetylated curcumin (AC-CU) with a 90% yield. Curcumin was hydrogenated using Pd-C so as to obtain a tetrahydrocurcumin (HCU) with a 45% yield. The hydrogenated curcumin was acetylated using acetic anhydride in the presence of a base so as to obtain diacetylated tetrahydrocurcumin (AC-THC) with a 75% yield.

### Biological Assays

### Quorum Sensing Inhibition Test With Bio-Reporter CV026 and KYC55

TLC preparation: all derivatives were dissolved in 100% acetonitrile to a concentration of 40 mM. Then placed 20 μlon the same spot on the TLC and let the solvent evaporate before use. CV026: few colonies were transferred to liquid LB (Lenox) for overnight incubation at 30° C. in a 45 degree in orbital shaker. At the O.D. 0.8 3 ml of the starter was added to preheated soft LB agar containing 500 nM of 3-oxo-C6 (Sigma-Aldrich). The mixture was then loaded on the TLC in a glass plate for O.N. incubation in 30° C. The next day inhibition zone was measured, while QS is taking place bacteria produce purple pigment, and when QS is inhibited no pigment is produced. KYC55 (pJZ372) (pJZ384) (pJZ410): 20 μl from the frozen stock into 25 ml AT media (AT buffer×20: 1.57 M KH₂PO₄ pH=7.3. AT salts mix×20: 300 mM (NH₄)₂SO₄, 13 mM MgSO₄, 1.4 mM CaCl₂, 0.4 mM FeSO₄·7H₂O, 0.26 mM MnSO₄·H₂O. AT media: 50 ml×20 AT-buffer, 50 ml×20 AT-salts, 10 ml 50% (w/v) glucose, add 890 ml to final volume of 1 liter.) For O.N. at 30° C. in an orbital shaker. Into preheated water agar with 25 ml AT-buffer, and 60 μg/ml X-gal and 5 nM 3-oxo-c8 (both from Sigma-Aldrich) 25 ml of the starter was added. Then, that mixture was loaded on the TLC in glass plate for O.N. incubation in 30° C. The next day the inhibition zone was measured. The bacteria produce blue pigment during QS and when QS is inhibited no pigment is produced. KYC55 (pJZ372) (pJZ384) (pJZ410) preservation in tetracycline (1 μg/ml), spectinomycin (100 μg/ml), gentamicin (100 μg/ml) (all antibiotics from Sigma Aldrich) in AT media O.N. then centrifuged and suspend in 15% glycerol frozen and maintained at −80° C.

### Diffusion Discs Test

All derivatives were dissolved in acetonitrile to a final concentration of 40 mM. Twenty (20) μl were loaded onto Whatman filter paper discs and dried. Bacillus subtilis, Staphylococcus aureus, and Acinetobacter baumannii were grown in LB (Lenox) O.N. One hundred (100) μl from the starter culture was plated on LB agar (Miller) dishes. Streptococcus sobrinus, and Streptococcus mutans grown in Brain Heary Infusion (BHI, Himedia) O.N. were similarly plated on BHI agar dishes. Discs were laid and incubated O.N. in 37° C. and the diameter of inhibition of bacterial growth was measured.

### NF-kB-Luciferase Reporter Gene Assay

L428 cells stable transfectants with the luciferase NF-kB-Luc reporter gene were generated previously described (Ozer et al., 2009), and were maintained in 500 μg/ml of G418 (G418 disulfate salt, Sigma-Aldrich). The cells (5×10 5 /well in triplicate) were incubated for 2 hrs. in 1 ml of medium containing the solvent (DMSO) or different concentrations of the molecules tested. Cells were then harvested, lysed, and monitored by a luciferase reporter assay kit (Promega) according to the manufacturer's instructions. Measurements were carried out using a luminometer (Promega, GLOMAX™ 20/20 Luminometer). Data were normalized to the protein concentration in each lysate as measured by the Bradford method (BioRad) and normalized to the control. Experiments were repeated at least three times. Initially, the inventors tested all derivatives that showed QSI activity at two concentrations: 80 μM and 160 μM (three independent experiments) for anti-inflammatory activity.

### Quantitation of Activated Nuclear p65 Fluorescence

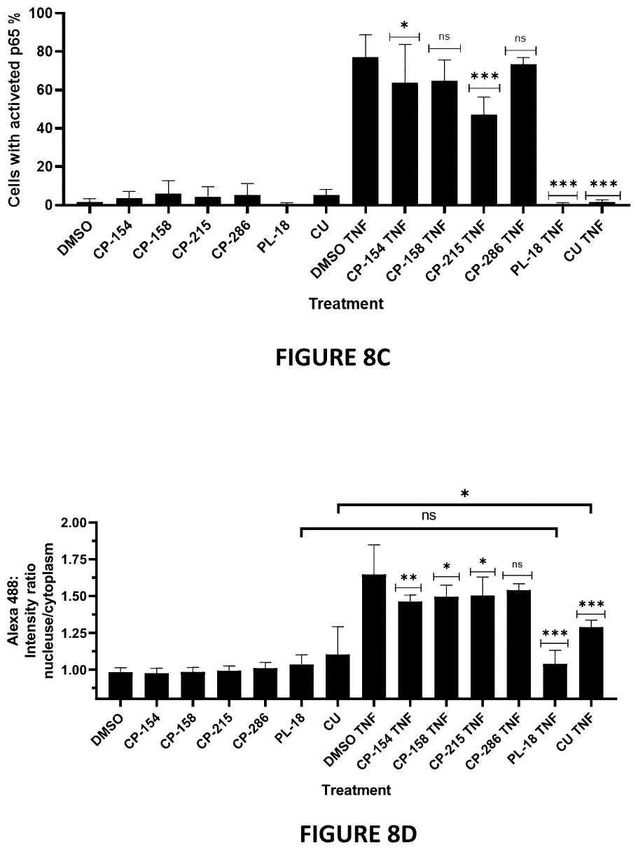

The A549 adherent human lung endothelial cells were grown in DMEM medium with 10% FBS, 1% Glutamine and Pen-Strep (all from Biological Industries, Israel), and carried by trypsinization (Trypsin EDTA solution B, Biological Industries, Israel). In contrast to L428 cells, NF kappa B inhibitor (IkB) is present in A549 cells. The inventors quantified the activated p65 NF-κB in the nucleus of the A549 cells as follows: 25,000 cells were plated per well of a 96 well plate (u-clear F-bottom, Greiner Bio-One) and allowed overnight attachment at 37° C., 5% CO₂. The derivatives (CP-154, CP-158, CP-215, CP-286 and Curcumin (CU) as positive control) were then added at the concentration of 160 μM for 2 hrs. One hundred and sixty (160) μM was chosen as the concentration which inhibited NF-kB in L428 cells according to the luciferase reporter gene assay. After 2 hrs., in order to fully activate NF-kB, TNFα (2.5 ng/ml Recombinant Human TNF-α Protein, R&D system) was added for additional 15 min. Then, the cells were washed twice with 3% FBS in PBS before being fixed with 4% paraformaldehyde (16% solution, EM Grade, Electron Microscopy Sciences) in PBS at room temperature for 20 minutes. Cells were then washed twice in PBS then twice in 3% FBS in PBS before permeabilization with 0.1% Triton X100 in PBS for 60 minutes at room temperature. Cells were then washed twice with 3% FBS in PBS before staining with NF-κB subunit P65 (mouse anti-p65 (F-6) SC-8008, Santa Cruz, USA) diluted 1:50 in 3% FBS and 0.1% Triton X100 in PBS overnight. The cells were then stained with a secondary antibody AF488 (green) (Goat anti-Mouse IgG (H+L) Alexa Fluor 488 [A-11029], Invitrogen, Thermo Fisher Scientific, Ma, USA) diluted 1:400 in 3% FBS and 0.1% Triton X100 in PBS overnight at RT. Nuclear staining with DAPI (SouthernBiotech, DAPI Fluoromount-G, blue-nuclear) was then performed for 30 min, followed by 3× washing with PBS. Cell fluorescence was imaged and quantitated by the Operetta High-Content Imaging System (Perkin Elmer) at 40× magnification. Analysis of the images obtained was done through the Columbus server of the company where parameters can be defined, such as cell area, nucleus, and cytoplasm separately. Different cell populations can be distinguished, and scoring was done on hundreds of cells in each treatment. As an example, see FIG. 8. Quantitative fluorescence intensity was averaged, and a relative fluorescence value was determined.

### Cell Viability by XTT Assay

The human Hodgkin's Lymphoma derived L428 cell line was used as a model. The cells were grown in RPMI Medium 1640 with 10% FBS, 1% Glutamine and pen-strep (all from Biological Industries, Israel). L428 cells do not express the NF-kB inhibitor (IkB). As a result, NF-kB is constitutively active and expressed mainly in the nucleus. Thirty thousand (30,000) cells/well were placed in 96-well plates with the coumaperine derivatives at different concentrations or with vehicle (DMSO) at a final volume of 200 μl. The plates were incubated for 48 hrs. at 37° C. and 5% CO₂ followed by the addition of 50 μl of the XTT reagent tetrazolium-formazan, (Biological Industries, Israel) was added for 2 hrs. at 37° C., 5% CO₂. In living cells, mitochondria oxidize the tetrazolium-formazan salt, thereby generating a color absorbing product at 450 nm. Absorbance was measured at 450 nm and 650 nm in an enzyme-linked immunosorbent assay (ELISA) plate reader (Multiskan Spectrum, Version 1.2), and recorded. Absorption values at 650 nm were subtracted from the blank and the 450 nm values. The results were normalized to the control (treated with DMSO). To determine the lethal concentration 50% (LC50) values of the derivatives, the Probit transformation was performed only for samples with R² values >0.85 and only on derivatives that showed significant cytotoxicity.

### Statistical Analysis

Two-way ANOVA followed by Tukey's multiple comparisons test or One-way ANOVA was performed using GraphPad Prism version 8.0.1 for Windows, GraphPad Software, San Diego, California USA, www.graphpad.com.

### EXAMPLE 1

### Synthesis of Mono, Di, and Triconjugated Coumaperine Derivatives

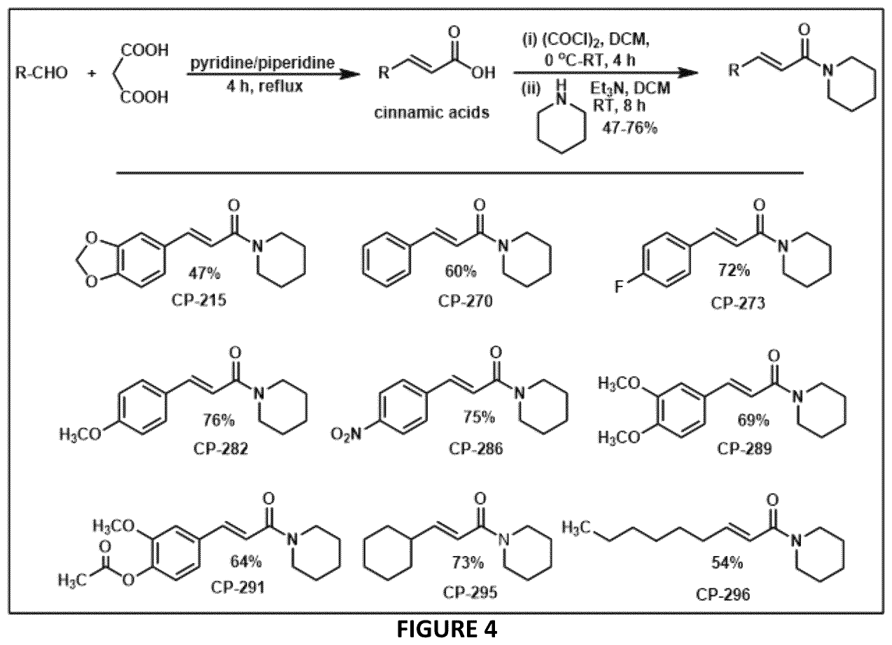

Monoconjugated coumaperine derivatives were synthesized in 47-76% yields (FIG. 4). CP-293 was prepared by deacetylation of acetyl derivative, CP-291 using pyrrolidine with 35% yield (FIG. 3).

Di/triconjugated coumaperine derivatives shown in FIG. 5 were synthesized in 12% to 99% yields. The synthesized di and triconjugated coumaperine derivatives are depicted in FIGS. 5-6. Coumaperine (CP) was acetylated to obtain CP-158 in moderate (36%) yield. For the present study, piperine was isolated from black pepper. Piperine was hydrogenated to obtain olefin saturated piperine, CP-THP. Piperine was deprotected using BBr₃ to obtain CP-209 in moderate yield (53%). CP-209 was also synthesized by other methods. N-Crotonyl piperidine was condensed with tetrahydropyran protected 3,4-dihydroxy benzaldehyde in presence of base (t-BuOK) to obtain tetrahydropyran protected CP-209. It was then deprotected with TFA to obtain CP-209 in moderated yield (50%). Acetylation of CP-209 with acetic anhydride in the presence of a base (Et₃N) gave the diacetylated product, CP-281-F1 in moderate yield (41%). CP-262-F1 and CP-262-F2 were synthesized from trimethoxy derivative, CP-27 (FIG. 5). CP-27 was demethylated using BBr₃ to obtain monodemthylated derivative, CP-262-F1 and tridemthylated derivative, CP-262-F2 in moderate to poor yields (33 and 14%) respectively. Attempt to isolate didemethylated derivative was not fruitful.

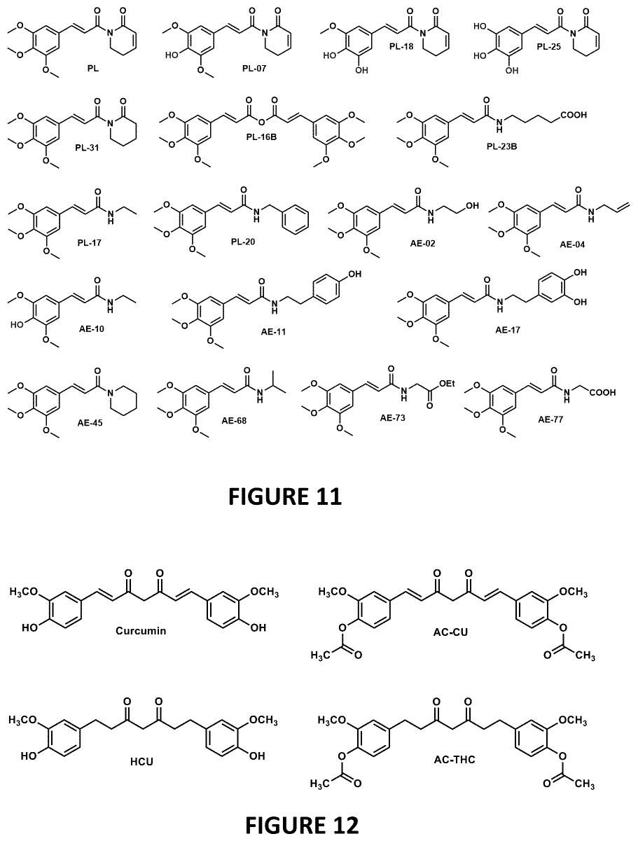

Piperlongumine derivatives (FIG. 11) were synthesized with yield % ranging from 32-83%.

Curcumin derivatives (FIG. 12) were synthesized with yield % ranging from 45-90%.

The synthesized compounds were characterized by various spectroscopic techniques including, FT-IR, ¹H NMR, ¹³C NMR, GC-MS, and HRMS/elemental analysis. The spectral data matched with structural attributes of the compounds.

### EXAMPLE 2

### Quorum Sensing Inhibition Assay

All the synthesized compounds were evaluated for quorum sensing inhibition against two bioreporter bacteria strains, Chromobacterium violaceum (CV026) and Agrobacterium tumefaciens (KYC55) and the results are summarized in Table 1.

The monoconjugated derivatives with simple phenyl group (CP-270), methylenedioxy substituted (CP-215) and alkyl-substituted compounds (CP-296) were highly active in both tests (Table 1, entries 1-3). The monomethoxy derivative, CP-282 (Table 1, entry 4) was more effective against CV026 than KYC55, while the dimethoxy derivative, CP-289 (Table 1, entry 5) was more effective against KYC55 than CV026. The dihydroxy compound, CP-237 and cycloalkyl derivative, CP-295 (Table 1, entries 6-7) displayed moderate activity against both the bacterial systems. The monoconjugated derivative with an electron-withdrawing substituent, nitro group (CP-286) exhibited low and moderate activity against CV026 and KYC55, respectively (Table 1, entry 8). CP-273 exhibited low activity against CV026 and inactive against KYC55. CP-291 was inactive against CV026 and exhibited moderate activity against KYC55. CP-293 was found to be inactive against both the bio-reporter bacterial strains (Table 1, entry 11).

The diconjugated derivative with fluorine substituent, CP-154 (Table 1, entry 12) was highly active in both tests. The acetylated coumaperine, CP-158 (Table 1, entryl3) was more effective against CV026 than KYC55. The vanillin derivative, CP-205 exhibited moderate activity against both the bacterial strains (Table 1, entry 14). The diacetylated (CP-281-F1), methoxy (CP-38) and simple phenyl derivatives, CP-9 (Table 1, entries 15-17) displayed moderate and low activity against CV026 and KYC55, respectively. CP-209 and CP-THP showed low activity against both bacterial strains (Table 1, entries 18-19). Coumaperine (CP), CP-32, 147 and 193 exhibited low activity and inactivity against CV026 and KYC55, respectively (Table 1, entries 20-23). CP-123 and 262-F2 were inactive against CV026 and displayed low activity against KYC55 (Table 1, entries 24-25). The other diconjugated derivatives such as CP-10, 27, 50, 184, 194, 209, 262-F1 and PIP were inactive against both the bacteria strains. The triconjugated derivative with the monomethoxy substituent, CP-102 (Table 1, entry 33) showed moderate and low QSI against CV026 and KYC55, respectively. The methylenedioxy substituted derivative (CP-155) and 3,4-dihydroxy derivatives (CP-239A) were inactive against both the bacterial strains (Table 1, entries 34-35).

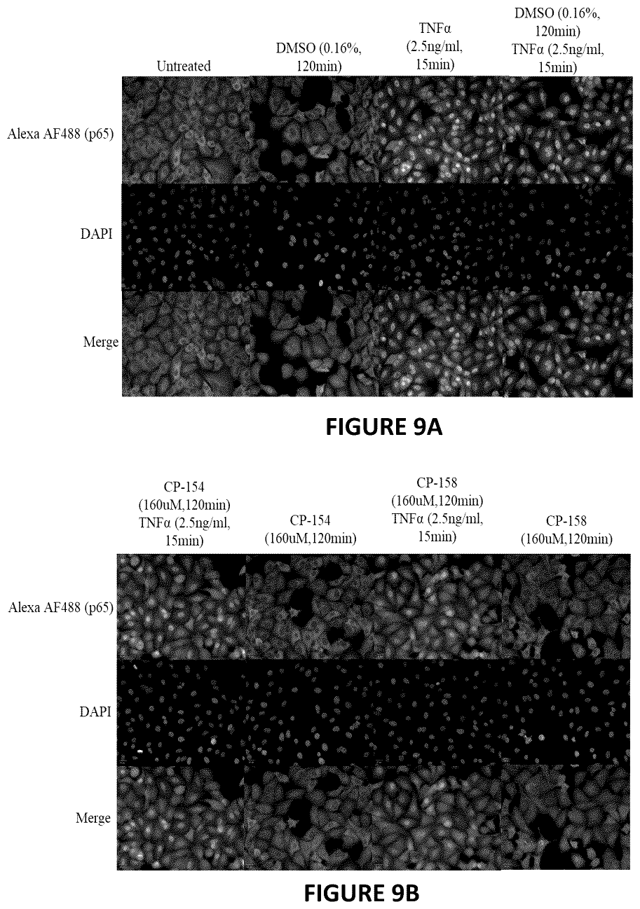

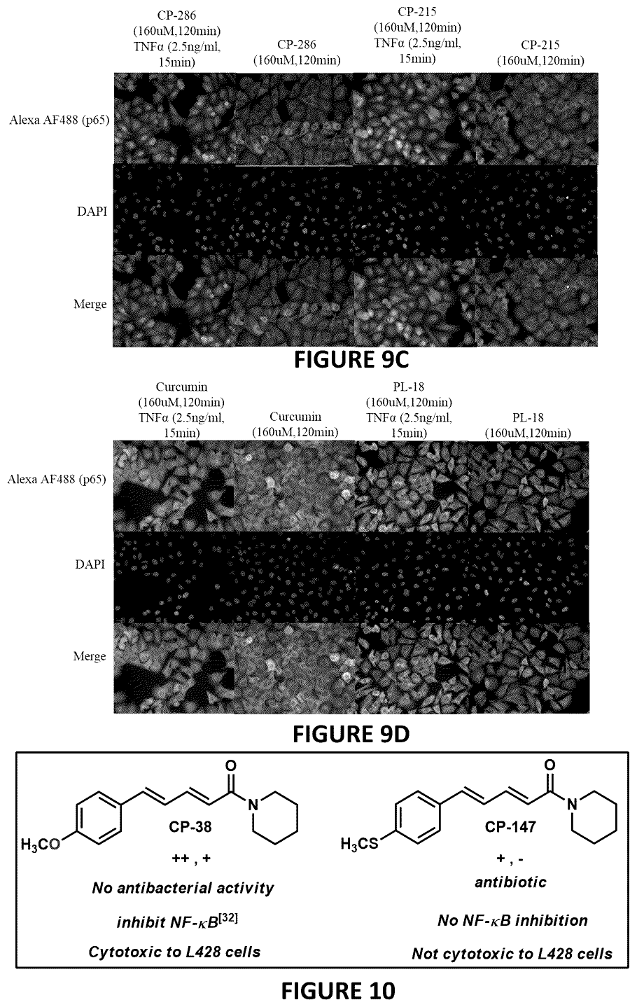

The anti-quorum sensing pattern depicted in Table 1 and FIG. 9 generally show that mono conjugated coumaperine derivatives were more effective in comparison to di and triconjugated derivatives against both bacterial strains, CV026 and KYC55. Thus, shorter conjugation may be more effective with most of the derivatives.

Furthermore, monoconjugated derivatives with simple alkyl, phenyl or aryl groups with electron-donating substituents were more effective than monoconjugated derivatives with electron-withdrawing substituents at the aryl group. The diconjugated derivatives with sterically less demanding electron-withdrawing groups, exhibited better anti-quorum sensing activity than the compounds with sterically bulky electron-withdrawing and electron-donating substituents. Similarly, the triconjugated derivatives with sterically less demanding electron-donating substituents were more effective than the derivatives with sterically bulky electron-donating substituents. Thus, monoconjugated derivatives with electron-donating and diconjugated with electron-withdrawing substituents exhibited a high anti-quorum sensing effect against both the bioreporter bacteria strains, CV026 and KYC55.

TABLE 1
Degree of quorum sensing inhibition (QSI) of coumaperine derivatives*

| | | Degree of QSI | Degree of QSI of |
|---|---|---|---|
| Entry | Monoconjugated-CP | of CV026 | KYC55 |
| 1 | 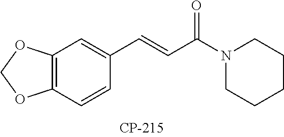 [SMILES: O=C(/C=C/c1ccc2c(c1)OCO2)N1CCCCC1] | +++ | +++ |
| 2 | 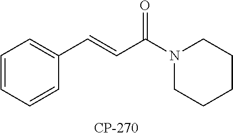 [SMILES: O=C(/C=C/c1ccccc1)N1CCCCC1] | +++ | +++ |
| 3 | 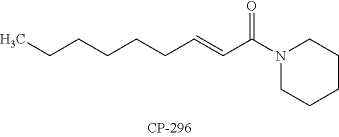 [SMILES: CCCCCC/C=C/C(=O)N1CCCCC1] | +++ | +++ |
| 4 | 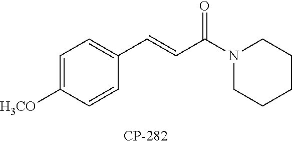 [SMILES: COc1ccc(/C=C/C(=O)N2CCCCC2)cc1] | +++ | ++ |
| 5 | 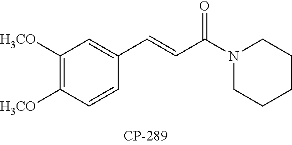 [SMILES: COc1ccc(/C=C/C(=O)N2CCCCC2)cc1OC] | + | +++ |
| 6 | 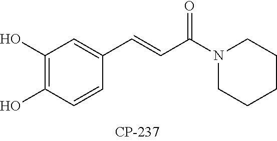 [SMILES: O=C(/C=C/c1ccc(O)c(O)c1)N1CCCCC1] | ++ | ++ |
| 7 | 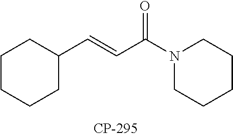 [SMILES: O=C(/C=C/C1CCCCC1)N1CCCCC1] | ++ | ++ |
| 8 | 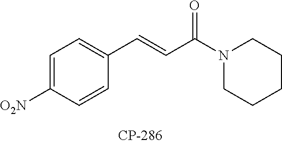 [SMILES: O=C(/C=C/c1ccc([N+](=O)[O-])cc1)N1CCCCC1] | + | ++ |
| 9 | 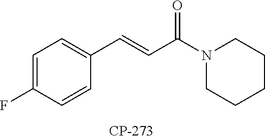 [SMILES: O=C(/C=C/c1ccc(F)cc1)N1CCCCC1] | + | − |
| 10 | 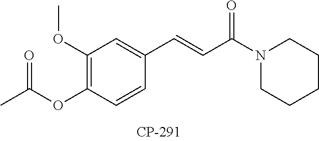 [SMILES: COc1cc(/C=C/C(=O)N2CCCCC2)ccc1OC(C)=O] | − | ++ |
| 11 | 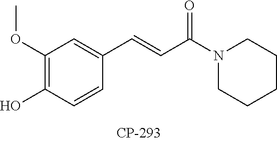 [SMILES: COc1cc(/C=C/C(=O)N2CCCCC2)ccc1O] | − | − |
| 12 | 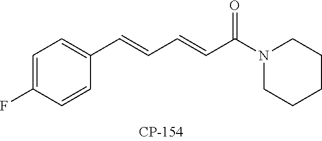 [SMILES: O=C(/C=C/C=C/c1ccc(F)cc1)N1CCCCC1] | +++ | +++ |
| 13 | 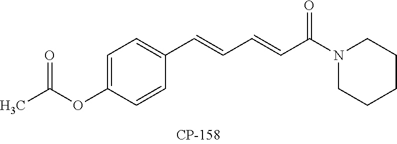 [SMILES: CC(=O)Oc1ccc(/C=C/C=C/C(=O)N2CCCCC2)cc1] | +++ | ++ |
| 14 | 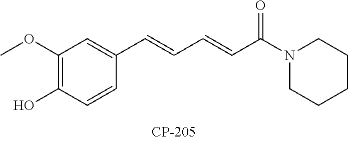 [SMILES: COc1cc(/C=C/C=C/C(=O)N2CCCCC2)ccc1O] | ++ | ++ |
| 15 | 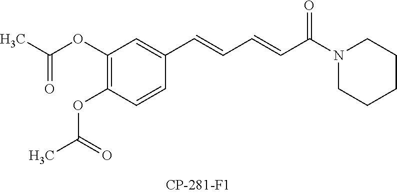 [SMILES: CC(=O)Oc1ccc(/C=C/C=C/C(=O)N2CCCCC2)cc1OC(C)=O] | ++ | + |
| 16 | 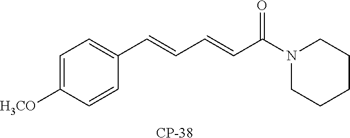 [SMILES: COc1ccc(/C=C/C=C/C(=O)N2CCCCC2)cc1 ; "CP-38"] | ++ | + |
| 17 | 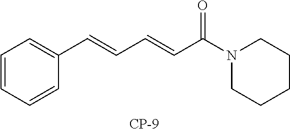 [SMILES: O=C(/C=C/C=C/c1ccccc1)N1CCCCC1 ; "CP-9"] | ++ | + |
| 18 | 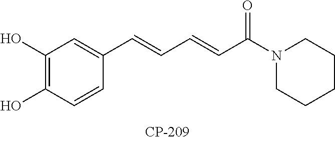 [SMILES: O=C(/C=C/C=C/c1ccc(O)c(O)c1)N1CCCCC1] | + | + |
| 19 | 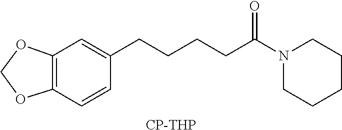 | + | + |
| 20 | 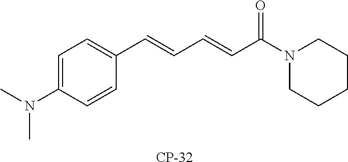 [SMILES: CN(C)c1ccc(/C=C/C=C/C(=O)N2CCCCC2)cc1 ; "CP-32"] | + | − |
| 21 | 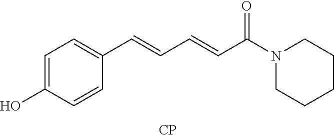 [SMILES: O=C(/C=C/C=C/c1ccc(O)cc1)N1CCCCC1 ; "CP"] | + | − |
| 22 | 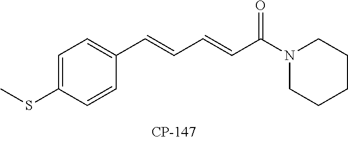 [SMILES: CSc1ccc(/C=C/C=C/C(=O)N2CCCCC2)cc1] | + | − |
| 23 | 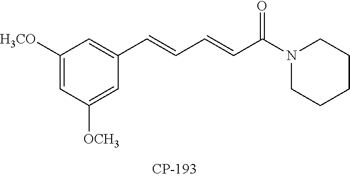 [SMILES: COc1cc(/C=C/C=C/C(=O)N2CCCCC2)cc(OC)c1] | + | − |
| 24 | 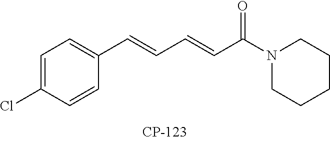 [SMILES: O=C(/C=C/C=C/c1ccc(Cl)cc1)N1CCCCC1] | − | + |
| 25 | 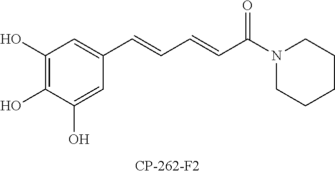 [SMILES: O=C(/C=C/C=C/c1cc(O)c(O)c(O)c1)N1CCCCC1] | − | + |
| 26 | 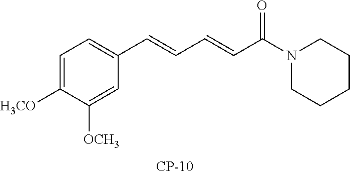 [SMILES: COc1ccc(/C=C/C=C/C(=O)N2CCCCC2)cc1OC ; "CP-10"] | − | − |
| 27 | 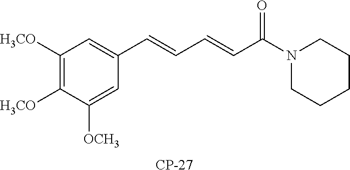 [SMILES: COc1cc(/C=C/C=C/C(=O)N2CCCCC2)cc(OC)c1OC ; "CP-27"] | − | − |
| 28 | 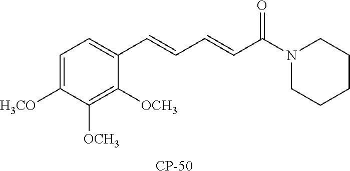 [SMILES: COc1ccc(/C=C/C=C/C(=O)N2CCCCC2)c(OC)c1OC ; "CP-50"] | − | − |
| 29 | 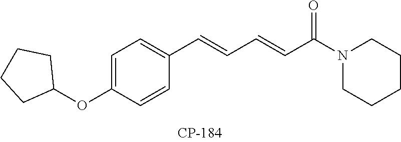 [SMILES: O=C(/C=C/C=C/c1ccc(OC2CCCC2)cc1)N1CCCCC1] | − | − |
| 30 | 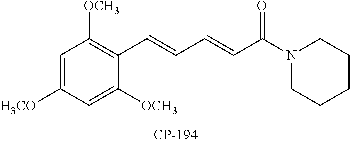 [SMILES: COc1cc(OC)c(/C=C/C=C/C(=O)N2CCCCC2)c(OC)c1] | − | − |
| 31 | 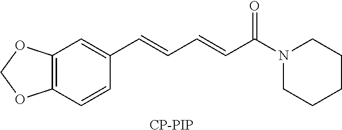 | − | − |
| 32 | 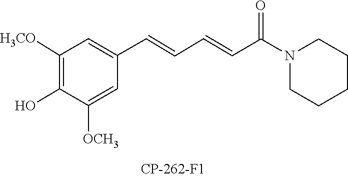 [SMILES: COc1cc(/C=C/C=C/C(=O)N2CCCCC2)cc(OC)c1O] | − | − |
| | | Degree of QSI | Degree of QSI of |
| Entry | Triconjugated-CP | of CV026 | KYC55 |
| 33 | 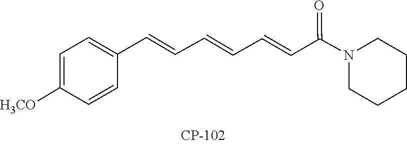 [SMILES: COc1ccc(/C=C/C=C/C=C/C(=O)N2CCCCC2)cc1] | ++ | + |
| 34 | 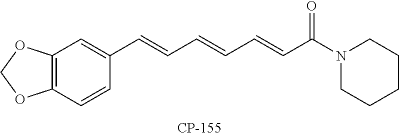 [SMILES: O=C(/C=C/C=C/C=C/c1ccc2c(c1)OCO2)N1CCCCC1] | − | − |
| 35 | 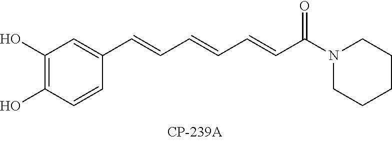 [SMILES: O=C(/C=C/C=C/C=C/c1ccc(O)c(O)c1)N1CCCCC1] | − | − |

*Two tests were carried out by using the reporter bacteria: Agrobacterium tumefaciens (KYC55) and Chromobacterium
violaceum (CV026). Bacteria were grown overnight in soft agar with the appropriate auto-inducer on a TLC plate where
the different compounds were dried on (40 mM in ~20 μL acetonitrile, and their degree of QSI was measured semi-
quantitatively by color intensity (− no inhibition, + low inhibition, ++ medium inhibition, +++ strong inhibition).
Experiments were repeated three times, results were determined by considering at least 2 out of 3 experiments with the same result.

Dihydroxy piperlongumine (PL-18) exhibits strong quorum sensing inhibition in KYC55 and moderate activity against CV026. The trihydroxy piperlongumine (PL-25) shows moderate inhibition against KYC55 and inactive against CV026. For PL-20, moderate activity is observed against CV026 and it is inactive against KYC55. PL-07 and 31 are inactive against KYC55 and show low activity against CV026. The parent compound PL and other derivatives such as PL-16A and PL-17 were inactive against both the bacterial strains.

TABLE 2
Degree of quorum sensing inhibition (QSI) of piperlongumine derivatives

| S. No | Structure | CV026 | KYC55 |
|---|---|---|---|
| 1 | 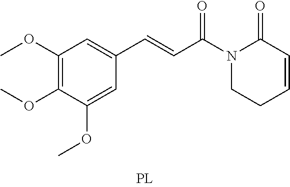 [SMILES: COc1cc(/C=C/C(=O)N2CCC=CC2=O)cc(OC)c1OC] | − | − |
| 2 | 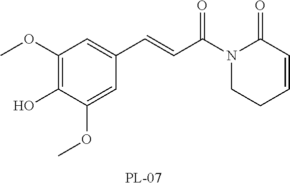 [SMILES: COc1cc(/C=C/C(=O)N2CCC=CC2=O)cc(OC)c1O] | + | − |
| 3 | 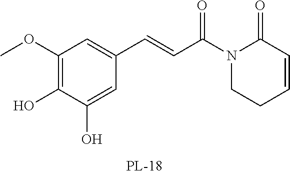 [SMILES: COc1cc(/C=C/C(=O)N2CCC=CC2=O)cc(O)c1O] | ++ | +++ |
| 4 | 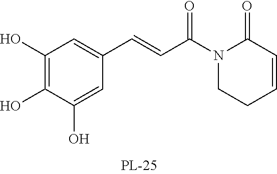 [SMILES: O=C1C=CCCN1C(=O)/C=C/c1cc(O)c(O)c(O)c1] | − | ++ |
| 5 | 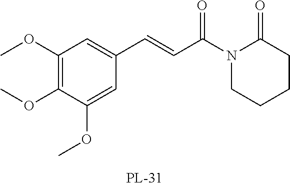 [SMILES: COc1cc(/C=C/C(=O)N2CCCCC2=O)cc(OC)c1OC] | + | − |
| 6 | 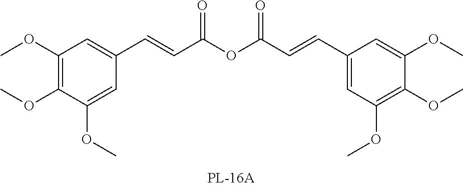 | − | − |
| 7 | 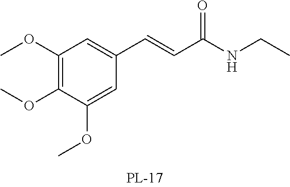 [SMILES: CCNC(=O)/C=C/c1cc(OC)c(OC)c(OC)c1] | − | − |
| 8 | 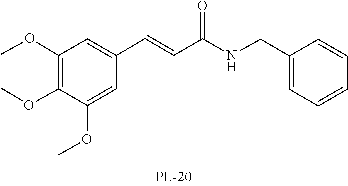 [SMILES: COc1cc(/C=C/C(=O)NCc2ccccc2)cc(OC)c1OC] | ++ | − |
| 9 | 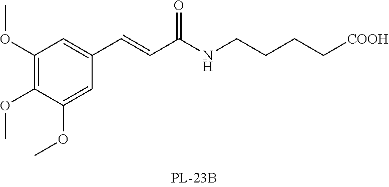 [SMILES: COc1cc(/C=C/C(=O)NCCCCC(=O)O)cc(OC)c1OC] | − | − |
| 10 | 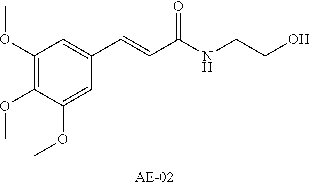 [SMILES: COc1cc(/C=C/C(=O)NCCO)cc(OC)c1OC] | − | − |
| 11 | 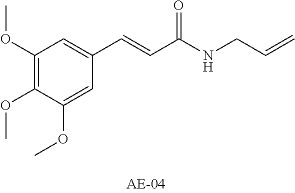 [SMILES: C=CCNC(=O)/C=C/c1cc(OC)c(OC)c(OC)c1] | − | + |
| 12 | 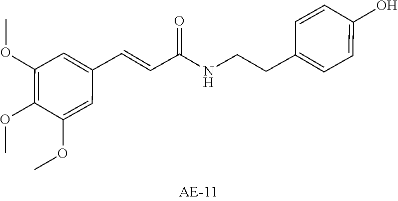 [SMILES: COc1cc(/C=C/C(=O)NCCc2ccc(O)cc2)cc(OC)c1OC] | − | − |
| 13 | 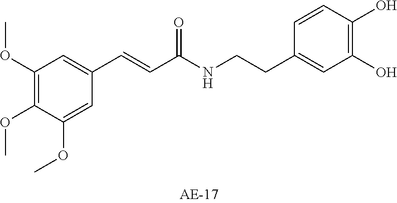 [SMILES: COc1cc(/C=C/C(=O)NCCc2ccc(O)c(O)c2)cc(OC)c1OC] | − | − |
| 14 | 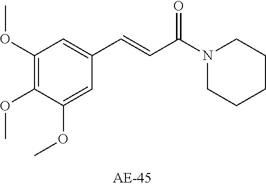 [SMILES: COc1cc(/C=C/C(=O)N2CCCCC2)cc(OC)c1OC] | − | − |
| 15 | 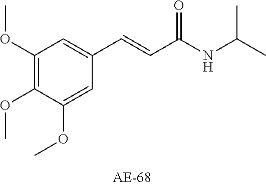 [SMILES: COc1cc(/C=C/C(=O)NC(C)C)cc(OC)c1OC] | − | − |
| 16 | 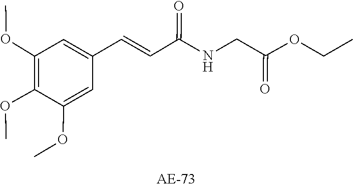 [SMILES: CCOC(=O)CNC(=O)/C=C/c1cc(OC)c(OC)c(OC)c1] | − | − |
| 17 | 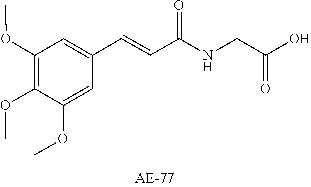 [SMILES: COc1cc(/C=C/C(=O)NCC(=O)O)cc(OC)c1OC] | − | + |
| 18 | 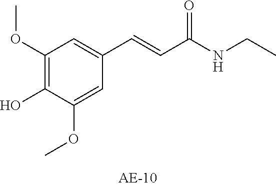 [SMILES: CCNC(=O)/C=C/c1cc(OC)c(O)c(OC)c1] | − | − |

The parent compound curcumin exhibited low activity against CV026 and inactive against KYC55. While the hydrogenated curcumin i.e., tetrahydrocurcumin showed strong activity against KYC55 and low activity against CV026. Both the acetylated curcumin (AC-CU) and acetylated tetrahydrocurcumin (AC-THC) were inactive against both the bio-reporter bacterial strains.

TABLE 3
Degree of quorum sensing inhibition (QSI) of curcumin derivatives

| S. No | Structure | CV026 | KYC55 |
|---|---|---|---|
| 1 | 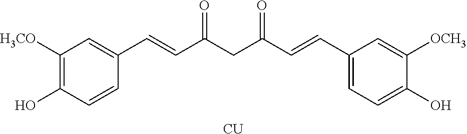 [SMILES: COc1cc(/C=C/C(=O)CC(=O)/C=C/c2ccc(O)c(OC)c2)ccc1O ; "CU"] | + | − |
| 2 | 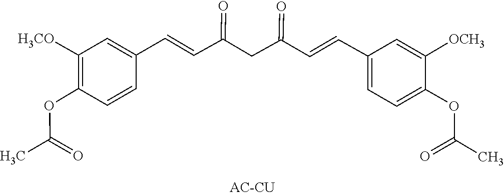 [SMILES: COc1cc(/C=C/C(=O)CC(=O)/C=C/c2ccc(OC(C)=O)c(OC)c2)ccc1OC(C)=O] | − | − |
| 3 | 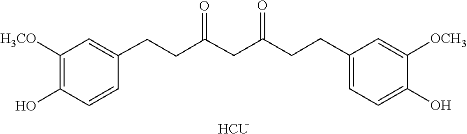 [SMILES: COc1cc(CCC(=O)CC(=O)CCc2ccc(O)c(OC)c2)ccc1O ; "HCU"] | + | +++ |
| 4 | 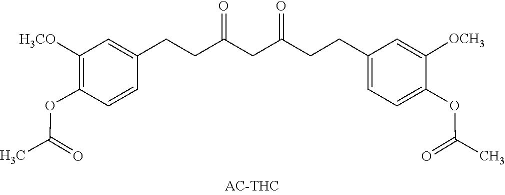 [SMILES: COc1cc(CCC(=O)CC(=O)CCc2ccc(OC(C)=O)c(OC)c2)ccc1OC(C)=O] | − | − |

### EXAMPLE 3

### Antibacterial Effect

The inventors also evaluated all the molecules that showed anti-quorum sensing activity for antibacterial properties test against both gram-positive and gram-negative bacteria. Interestingly, none of the molecules showed antibacterial action against gram-negative bacteria and only five of the coumaperine derivatives: CP-9, CP-154, CP-147, CP-295 and CP-287 and one of the piperlongumine derivative PL-25 showed antibacterial activity, exclusively against the Gram-positive bacteria. The antibacterial activities of derivatives showing QSI are outlined in Table 4. These results show that coumaperine derivatives that have the potential to inhibit QS but are not antibiotic, could be used, avoiding the risk of selecting for drug-resistant bacteria. Similar to that idea, synthetic derivatives of furanon (product of marine algae) shown in-vivo QSI activity without antibacterial activity followed by immune response of the infected animal. Phenolic compounds decrease QS and virulence in PA01 and Chromobacterium violaceum. There is increasing number of studies, searching for compounds that targeting non-vital processes to combat bacteria.

TABLE 4
Antibacterial activity of coumaperine derivatives*

| | Bacillus | | Streptococcus | | | |
|---|---|---|---|---|---|---|
| Compound | subtilis | Staphylococcus | sobrinus | Streptococcus | PA01 | Acinetobacter |
| code | (mm) | aureus (mm) | (mm) | mutans (mm) | (mm) | baumannii (mm) |
| CP-147 | — | 9 | — | — | — | — |
| CP-154 | 7 | 11 | 9 | — | — | — |
| CP-9 | 8 | 9 | 9 | — | — | — |
| CP-295 | — | — | 12 | — | — | — |
| CP-287 | — | — | 9 | — | — | — |
| PL-25 | — | 9 | — | — | — | — |

*Discs with 40 mM of compound in 20 μl solvent (DMSO) were placed on petri dishes with different bacteria.
The numbers represent the diameter of growth inhibition (mm).
(—), no activity.

### EXAMPLE 4

### NF-kB Luciferase Reporter Gene Assay

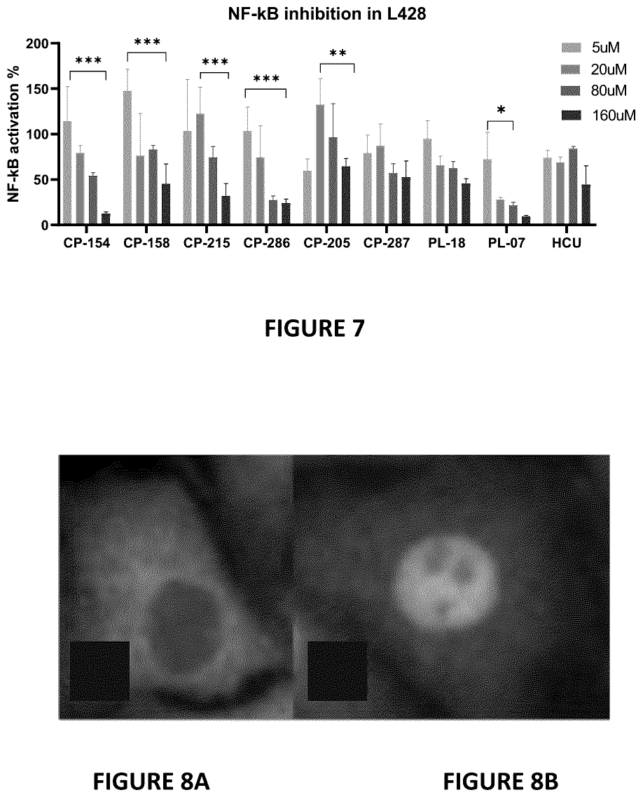

The compounds that showed anti-inflammatory and/or anti-QS activity were further investigated to determine their potency in a dose response experiment and are presented in FIG. 7. At the examined concentrations, the compounds efficiently inhibited NF-kB in L428 cells.

It is of importance to develop anti-inflammatory drugs that inhibit NF-kB, to ameliorate or prevent disease. It has been shown that the QS molecule, 3-oxo-C12 (PA01) temporarily inhibits the de-novo synthesis of IkB, the inhibitor of NF-κB, enabling constitutive NF-kB activation and inflammation, suggesting that QS and the inflammatory response in the host, have a symbiotic co-modulatory relationship. Thus, it is possible that anti-QS molecules can up- or down-modulate the inflammatory response providing potential therapeutic use. To further explore the possibility of QS molecules as modulators of inflammation the inventors also evaluated the effect of the anti-QS active compounds in reducing inflammation by luciferase reporter gene NF-kB inhibition. Except for CP-158, the other compounds (CP-154, 215 and 286) decreased NF-kB activation to less than 30%. Suggesting that these compounds could be potential anti-inflammatory candidates. PL-07 and PL-18 were also found to decrease NF-kB as well. CP-287 and CP-205 showed mild NF-kB inhibitory activity, and HCU, a derivative of curcumin have showed moderate inhibitory effect on NF-kB (FIG. 7).

### EXAMPLE 5

### Quantitation of Activated Nuclear p65-NF-kB Fluorescence

To confirm that the derivatives (CP-154, CP-158, CP-286 and CP-215) inhibit NF-kB as detected by the luciferase reported gene assay, the inventors used immunostaining. A549 cells were treated with different derivatives, CP-154, CP-158, CP-286 and CP-215 followed by activation of NF-kB with TNFα. Fluorescent images were taken from 25 fields in each well in duplicate by the Operetta imaging system and analyzed through the Columbus server. The inventors have visualized the differences between inactive p65 (FIG. 8A) and active p65 followed by TNFα incubation (FIG. 8B). To analyze these results, the inventors defined the active population in the Columbus server (FIG. 8C) and the relative fluorescence values of the nucleus and cytoplasm (FIG. 8D). As shown in FIG. 8, CP-154, and CP-215 significantly inhibited NF-kB activated by TNFα, while CP-286 and CP-158 were less active (FIG. 8D). The same results were obtained when the percentage of cells with active NF-kB were scored (FIG. 8C).

In principle, the results disclosed herein confirm those obtained by the luciferase reporter gene system. CP-286 and CP-158 were less effective in A549 as compared to their effect in L428 cells. The difference may be due to the compounds' different mechanisms of action, independent or dependent on IkB, since L428 cells (luciferase reporter gene) lack IkB, while present in A549 (immunostaining) cells.

### EXAMPLE 6

### Cell Viability Assay

The inventors further tested the cytotoxicity of the compounds that showed QSI on L428 cells as described in Materials and Methods. CP-205, CP-286, CP-273, CP-296, CP-281-F1, CP-289, CP-270, CP-282 and CP-215 were found to have no cytotoxic activity at the concentrations tested. CP-9, CP-154 and CP-38 were found to have a toxic effect with an LC50 of 24.70 μM, 46.78 μM and 47.44 μM, respectively. CP-158 and CP-295 were found to have cytotoxic effect yet to somewhat less extent, with LC50 of 285.38 μM, and 408.27 μM, respectively. Likewise, CP-287 was shown to have a LC50 of 283.8 μM. HUC was shown to have an LC50 of 101.52 μM. This LC50 is well within the concentration range that was examined in the other activity assays (e.g., NF-kB, and luciferase reporter assay).

XTT viability test were also performed, and the results are summarized in table 5, hereinbelow. Viability was determined in triplicate in two independent tests. The results were normalized to vehicle (DMSO) treated cells, (100% viability). Mean+SD.

TABLE 5
XTT assay - results summary

| Compounds code | LC50 (μM) | Compounds code | LC50 (μM) |
|---|---|---|---|
| CP-215 | Non-toxic | CP-205 | Non-toxic |
| CP-273 | Non-toxic | CP-287 | 283.8 |
| CP-296 | Non-toxic | PL-20 | Non-toxic |
| CP-281F1 | Non-toxic | HCU | 101.52 |
| CP-286 | Non-toxic | PL-25 | 46.33 |
| CP-289 | Non-toxic | PL-18 | 9.3 |
| CP-270 | Non-toxic | | |
| CP-282 | Non-toxic | | |
| CP-295 | 408.27 | | |
| CP-158 | 285.38 | | |
| CP-38 | 47.44 | | |
| CP-154 | 46.78 | | |
| CP-9 | 24.71 | | |

### Conclusions

Finding molecules that inhibit QS and inflammation without having antibacterial action is of utmost importance. Agents that interfere with bacterial virulence may prevent the selection of antibiotic-resistant bacteria, conceivably will have important therapeutic applications. In the present study, coumaperine, which resembles piperine in structure and is present in low concentrations (<6 ppm) in white pepper, as well as its synthetic derivatives, were evaluated as quorum sensing inhibitors. Although coumaperine is characterized by important pharmacophores such as the Michael acceptor, phenolic and amide moieties, its bioactivity has not been properly explored, probably due to its low bioavailability and long synthetic protocols. Recently, the inventors reported a simple and a robust methodology for the synthesis of coumaperine and its derivatives. Following that synthetic process, coumaperine derivatives depicted in FIGS. 3-6, were synthesized, and characterized for the present study. Initially, the synthesized compounds were evaluated for their QSI properties. These compounds were then examined further for their antibacterial activity, cytotoxicity, and anti-inflammatory activity.

The quorum sensing inhibitory potential of all the coumaperine derivatives was evaluated. CV026 and KYC55 bacteria detect acyl-homoserine-lactone (AHL), a common signal molecule known as autoinducer (AI) of gram-negative bacteria. Different AIs differ in the carbon chain length of the acyl group. CV026 detect N-hexanoyl-L-homoserine lactone (HHL) and represent AIs with a shorter carbon chain, whereas KYC55 detect N-3-oxooctanoyl-HSL (OOHL) represents AIs with longer carbon chains. Together, these two QS systems are sensitive to a wide range of AIs and serve as sensitive screen models to a wide range of inhibitory molecules.

Of the tens of compounds disclosed herein, 5 compounds (CP-9, CP-154, CP-158, CP215 and CP-286) were found to have both anti QS activity and an anti-inflammatory ability. Out of these 5 specific compounds, three were found to be not cytotoxic and did not show antibiotic activity (CP-158, CP-215 and CP-286).

The summary of the activities for CP-9, CP-154, CP-158, CP-215,CP-286, CP-287, CP-205, PL-18, and HUC is presented in Table 6. CP-9 was previously reported to be cytotoxic and to inhibit NF-1c13. Here, the inventors show that CP-9 also has a moderate and low inhibitory effect on QS of CV026 and KYC55, respectively (Table 1) and inhibit bacterial growth of the three gram-positive bacteria were tested (Table 4). All these activities suggest that CP-9 has no specific target but may affect multiple targets within bacterial or human cells. Similarly, CP-154 also shows multiple activities. Importantly, of these compounds, CP-215 is active but not cytotoxic showing promise as a relatively safe QSI molecule (Table 1). PL-18, is a potent piperlongumine derivative, characterized by having moderate QSI and anti-inflammatory activities. HCU was also found to be cytotoxic, as it induced a toxic effect on the viability of L428 cells (at 48 hr).

TABLE 6
Summary of Biological activities*

| Compound code | QSI | Antibacterial | Anti-inflammatory | Cytotoxic |
|---|---|---|---|---|
| CP-9 | + | + | + | + |
| CP-154 | + | + | + | + |
| CP-158 | + | − | + | + |
| CP-215 | + | − | + | − |
| CP-286 | + | − | + | − |
| CP-287 | + | − | + | + |
| CP-205 | + | − | + | − |
| PL-18 | + | − | + | + |
| HCU | + | − | + | + |

*Five compounds with QSI and anti-inflammatory properties were tested for antibacterial and cytotoxic activities.
(+) active (−) inactive.

The structure of the derivatives mainly differs in the degree of conjugation and/or in the functional group. Some structural features have impact on the QSI and NF-kB inhibition activities. For example, CP-273 and CP-154 have a similar structure, containing fluoride and differing only in their carbon chain length. Despite their similarity, they perform very differently in their biological activities. CP-273 has low QSI, no anti NF-kB activity (Table 1) and is not cytotoxic. CP-154 on the other hand showed activity but was also cytotoxic. The major differences in their activity might have been determined by the length of their carbon chain. Furthermore, CP-215, PIP and CP-155 differ only in their carbon chain length but showed varied biological activity. CP-215 has strong QSI and anti-inflammatory activity (Table 1, FIGS. 7-8). By contrast, PIP and CP-155 do not inhibit QS or NF-kB. The inventors also compared CP-270, a mono-conjugate derivative and CP-9, a di-conjugate derivative with no functional group on their aromatic ring. While CP-9 had some antibiotic activity CP-270 did not. CP-270 is highly QSI active in both bio-reporters but CP-9 has moderate and low activity in CV026 and KYC55, respectively (Table 1). In this study, CP-270 had no anti-inflammatory activity, while CP-9 demonstrated strong NF-kB inhibition. Similarly, CP-293, a mono-conjugated derivative and CP-205, a di-conjugated derivative with vanillin skeleton showed varied activity. CP-205 is moderately active against both bacterial strains whereas CP-293 is inactive on both the strains. Interestingly, curcumin is inactive against KYC55, but its saturated version (HCU) exhibits strong activity. Suggesting that reduced conjugation and alkyl hydrophobic environment favors strong activity.

The unique contribution of the functional group can be observed in the couple CP-38 and CP-147, both di-conjugates, where the differences in their structure area thiomethyl functional group (SCH3) and in CP-38 a methoxy (OCH 3) moiety. This relatively small change has a significant impact on their functions, whereas CP-147 inhibits Staphylococcus aureus growth (Table 2), has a low QS inhibition effect on CV026, does not affect QS in KYC55 (Table 1), and has no effect on NF-kB. The activities of CP-38 were listed above (and summarized in FIG. 10). The different functional group among them is interesting, where oxygen is more active than thio at this position.

In this study, the inventors showed that synthetic derivatives of natural compounds often have improved therapeutic value over their natural precursor compounds, e.g., in terms of solubility, toxicity, activity, and specificity.

### EXAMPLE 7

### Inhibition of P. aeruginosa QS

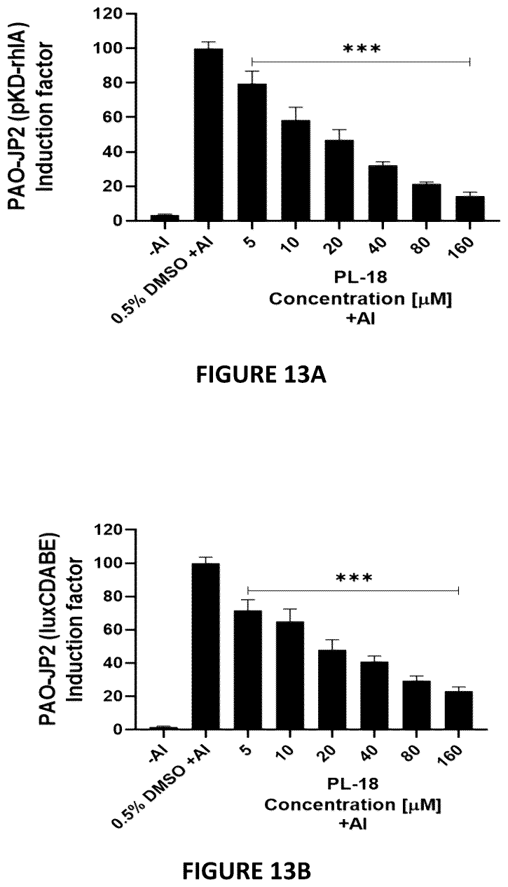

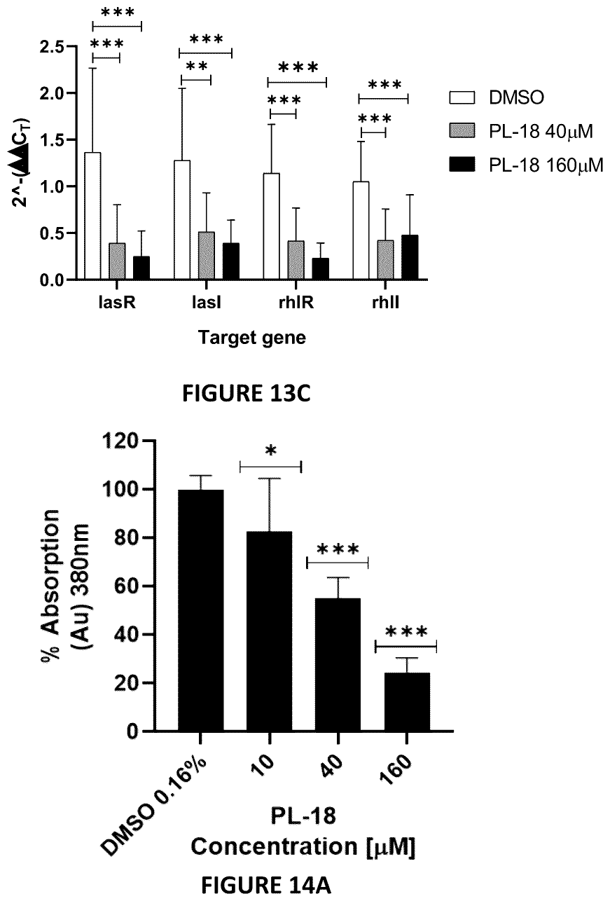

PL-18 was shown to inhibit two major P. aeruginosa QS systems in a dose dependent manner. The inhibition of rhlR-rhlI system was slightly better than the inhibition of lasR-lasI. Inhibition of rhlR-rhlI was 85.6% at 160 μM with IC₅₀ of 18 04. lasR-lasI inhibition was 76.85% at 160 μM with a similar IC₅₀ value of 22 μM (FIGS. 13A-13B). The relative transcription levels of QS genes lasR, lasI, rhlR and rhlI, was determined by RT-qPCR. The results show that PL-18 strongly decreased the level of all the genes tested (FIG. 13C).

PL-18 was shown to inhibit four different gram-negative bacterial HSL systems, corresponding to different AI molecules; CV026-3-oxo-C6-HSL, KYC55-3-oxo-C8-HSL, P. aeruginosa-3-oxo-C12-HSL and C4-HSL. The corresponding AI receptors are homologous proteins to the LuxR family that respond to different AIs which differ in the carbon chain length, saturation, and substitution on the third carbon (oxo or hydroxyl). The degree of specificity of the receptors varies and depends on these parameters (Wellington and Greenberg 2019). Interestingly, the ability of PL-18 to non-selectively inhibit several HSL receptors suggests that size factor may be important enabling PL-18 interact with diverse receptors. The molecular mass of PL-18 is 289.28 gr/mol. Similarly, other effective QSI molecules have also small molecular mass, such as halogenated furanone, molecular weight of 258.88 gr/mol (Hentzer et al., 2002), and Hordenine analogs in the range of 165 gr/mol (Liu et al., 2021). Furthermore, PL-18 most likely does not inhibit the synthesis of the AI since the reporter strains of P. aeruginosa (PAO-J2), A. tumefaciens (KYC55) and C. violaceum (CV026) are null mutants of the endogenous AI synthase, resulting in lack of AI in these bacteria.

The transcription levels of the QS genes, PA01, lasR, lasI, rhlR and rhlI were down regulated. The data corroborates with the current results with reporter bacteria, showing inhibition of lasR and rhlR. These receptors coupled with their AIs act as transcription factors which activate their own gene expression along with other genes (Whitehead et al., 2001).

When inhibition of QS was tested, it was observed that when the methyl moiety was substituted by a hydroxyl, QS was inhibited. Two or three hydroxyls correlated with better QSI activity: PL-18, PL-25>PL-20>PL-07, PL-17, and PL-31. PL was inactive.

### EXAMPLE 8

### Attenuation of P. aeruginosa Virulence

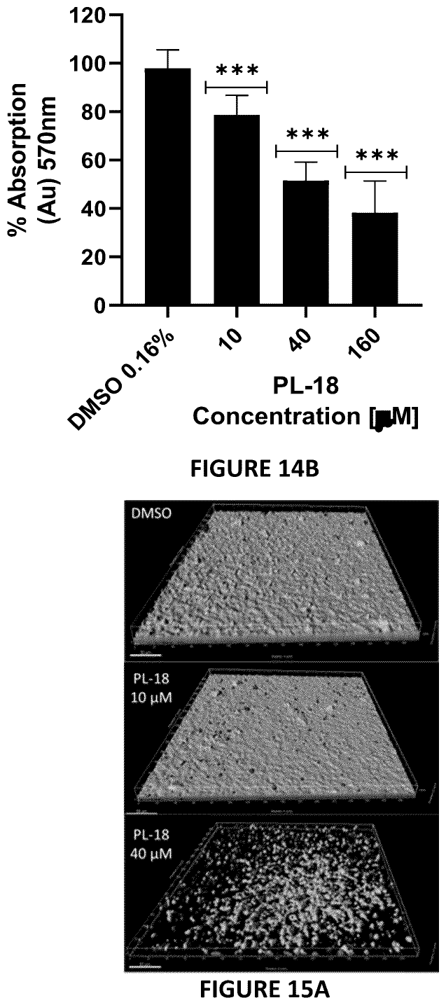

To test whether PL-18 inhibits virulence effectors secreted by P. aeruginosa, the relative abundance of the virulence factors pyocyanin and rhamnolipids in the bacterial cell-free culture medium was determined by spectrometry. PL-18 was shown to inhibit both virulence factors in a dose dependent manner (FIG. 14). Nonetheless, PL-18 was not shown to inhibit any one of: the secretion of the protein elastase, another virulence factor, and motility of P. aeruginosa (data not shown).

The inventors also determined the toxicity of PL-18 to P. aeruginosa. Viability was tested by scoring CFUs of PL-18 treated P. aeruginosa cultures and bacterial growth was determined by incubation with PL-18 for kinetic O.D.₆₀₀ₙₘ measurements. PL-18 was found to be non-toxic and did not affect P. aeruginosa growth at the concentrations tested (data not shown).

### EXAMPLE 9

### Attenuation of P. aeruginosa Biofilm

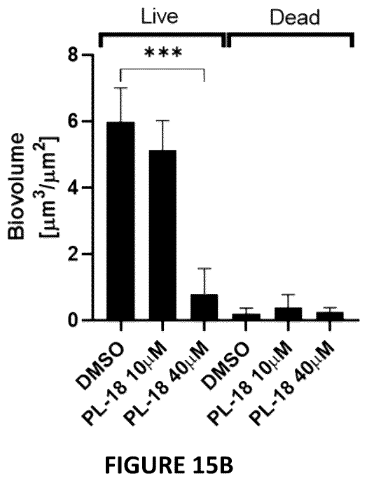

The anti-biofilm activity of PL-18 was tested in flow conditions for 72 hours. The biofilm was visualized by CLSM and quantified using the IMARIS software. PL-18 effectively inhibited (85%) P. aeruginosa biofilm formation at 40 μM (FIG. 15).

It is known that the QSI activity decreases the virulent behavior of P. aeruginosa (Rasmussen et al., 2005). Indeed, PL-18 was shown to effectively attenuate secretion of rhamnolipids, pyocyanin, and most importantly P. aeruginosa biofilm formation. However, PL-18 was not found to inhibit motility and elastase secretion (data not shown).

To conclude, QS inhibition is a promising approach to prevent bacterial virulence, focused on the disturbance of bacterial communication. Coupled with the anti-inflammatory properties of the herein disclosed derivatives, these molecules are promising candidates for developing therapeutic substances to prevent complications resulting from bacterial infections.

### EXAMPLE 10

### Synthetic Procedures for the Exemplary Compounds of the Invention

### Synthesis of (E)-3-(benzo[d][1,3]dioxol-5-yl)-1-(piperidin-1-yl)prop-2-en-1-one (CP-215)

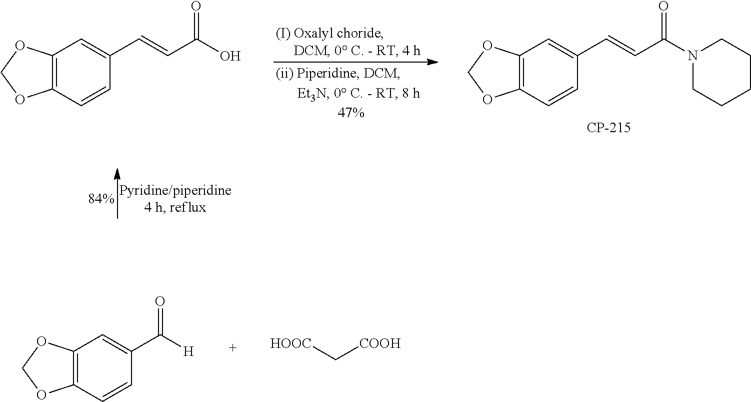 [SMILES: O=C(O)/C=C/c1ccc2c(c1)OCO2 ; O=C(/C=C/c1ccc2c(c1)OCO2)N1CCCCC1 ; O=Cc1ccc2c(c1)OCO2 ; O=C(O)CC(=O)O]

To a solution of piperonal (1.0 g, 6.66 mmol) in pyridine (10 ml) at room temperature, malonic acid (1.38 g, 13.3 mmol) was added and stirred for 10 minutes to this 0.5 mL of piperidine was added and then the reaction mixture was refluxed for 4 h. The progress of the reaction was monitored by TLC (EtOAc). After completion, the reaction mixture was quenched with 1M HCl (20 mL). The product precipitated as white crystalline solid. It was washed with dil. HCl (25 mL), diethyl ether (10 mL) and dried under vacuum to get (E)-3,4-(methylenedioxy)cinnamic acid in 1.07 g (84% yield).

To a cold (0° C.) solution of (E)-3,4-(methylenedioxy)cinnamic acid (1 g, 5.2 mmol) in DCM under N₂ atmosphere, oxalyl chloride (1.3 g, 10.3 mmol) was added slowly and the reaction mixture was slowly warmed to room temperature and stirred for 4 h. Excess oxalyl chloride was distilled off. Without further purification, the crude product was taken to the next step.

To a stirred solution of piperidine (0.44 g, 5.1 mmol) and Et₃N (0.57 g, 5.6 mmol) in DCM (20 mL) at 0° C., the above-synthesized acid chloride in DCM was added dropwise. After the addition, the reaction mass was slowly warmed to room temperature and stirred for 8 h. Progress of the reaction was monitored by TLC (hexane-EtOAc (60:40)). After completion, the DCM layer was washed with sat. NaHCO₃ solution (100 mL), water (2×75 mL), dried over anhyd. Na₂SO₄ and concentrated under reduced pressure. The obtained crude product was column purified to get the pure product as off-white solid in 0.63 g (yield 47%).

### Synthesis of (E)-3-(3,4-dihydroxyphenyl)-1-(piperidin-1-yl)prop-2-en-1-one (CP-237)

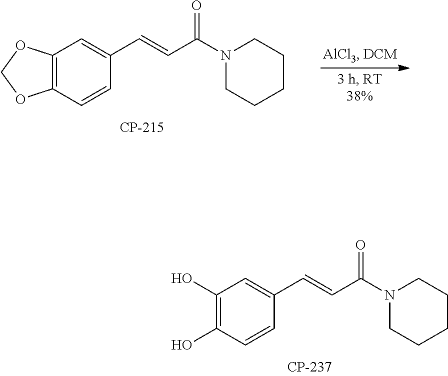 [SMILES: O=C(/C=C/c1ccc2c(c1)OCO2)N1CCCCC1 ; O=C(/C=C/c1ccc(O)c(O)c1)N1CCCCC1]

To a suspension of AlCl₃ (1.28 g, 9.6 mmol) in DCM (10.0mL) at room temperature, a solution of CP-215 (0.5 g, 1.9 mmol) in DCM (10.0 mL) was added dropwise under N₂ atm. Stirring continued for 3 h at room temperature. After completion, the reaction mixture was cooled to 0° C., quenched with 1% HCl solution (50 mL) and poured into sat. brine solution (50 mL), extracted with EtOAc (2×100 mL), the combined organic layer was washed with sat. brine solution (50 mL), dried over Na₂SO₄ and concentrated under reduced pressure to get the crude product. The pure product was obtained after column purification as off-white solid in 0.18 g (yield 38%).

### Synthesis of (E)-3-(benzo)-1-(piperidin-1-yl)prop-2-en-1-one (CP-270)

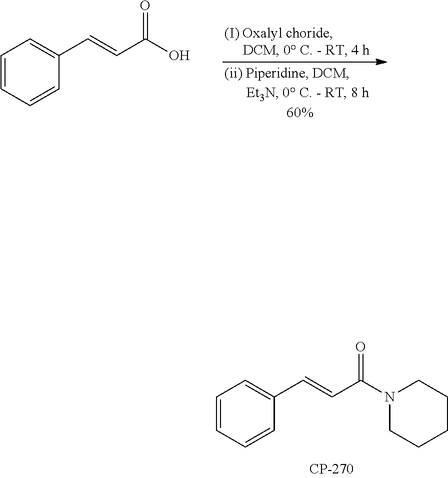 [SMILES: O=C(O)/C=C/c1ccccc1 ; O=C(/C=C/c1ccccc1)N1CCCCC1]

To a cold (0° C.) solution of (E)-cinnamic acid (1 g, 6.7 mmol) in DCM under N₂ atmosphere, oxalyl chloride (1.28 g, 10.1 mmol) was added slowly and the reaction mixture was slowly warmed to room temperature and stirred for 4 h. Excess oxalyl chloride was distilled off. Without further purification, the crude product was taken to the next step.

To a stirred solution of piperidine (0.56 g, 6.6 mmol) and Et₃N (0.73 g, 7.2 mmol) in DCM (20 mL) at 0° C., the above-synthesized acid chloride in DCM was added dropwise. After the addition, the reaction mass was slowly warmed to room temperature and stirred for 8 h. Progress of the reaction was monitored by TLC (hexane-EtOAc (60:40)). After completion, the DCM layer was washed with sat. NaHCO₃ solution (100 mL), water (2×75 mL), dried over anhyd. Na₂SO₄ and concentrated under reduced pressure. The obtained crude product was column purified using hexane-EtOAc (90:10) to get the pure product as off-white solid in 0.87 g (yield 60%).

### Synthesis of (E)-3-(benzoyl 4-fluoro)-1-(piperidin-1-yl)prop-2-en-1-one (CP-273)

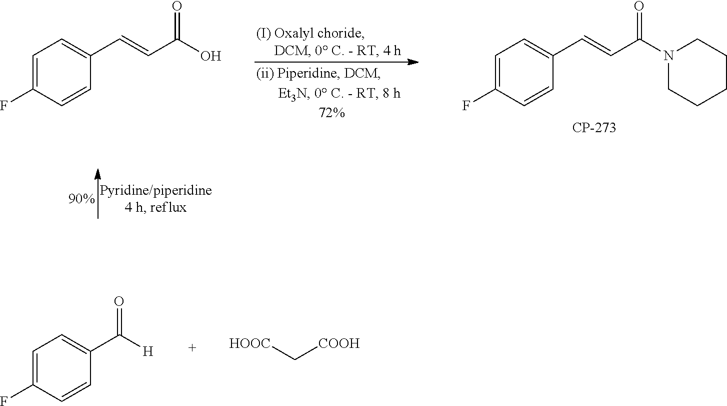 [SMILES: O=C(O)/C=C/c1ccc(F)cc1 ; O=C(/C=C/c1ccc(F)cc1)N1CCCCC1 ; O=Cc1ccc(F)cc1 ; O=C(O)CC(=O)O]

To a solution of 4-fluorobenzaldhyde (1.0 g, 8.06 mmol) in pyridine (10 ml) at room temperature, malonic acid (1.67 g, 16.1 mmol) was added and stirred for 10 minutes, to this 0.5 ml of piperidine was added and then the reaction mixture was refluxed for 4 h. The progress of the reaction was monitored by TLC (EtOAc). After completion, the reaction mixture was quenched with 1M HCl (20 mL). The product precipitated as white crystalline solid. It was washed with dil. HCl (10 mL), diethyl ether (10 mL) and dried under vacuum to get (E)-4-fluorocinnamic acid in 1.20 g (90% yield).

### Synthesis of (E)-3-(benzoyl 4-methoxy)-1-(piperidin-1-yl)prop-2-en-1-one (CP-282)

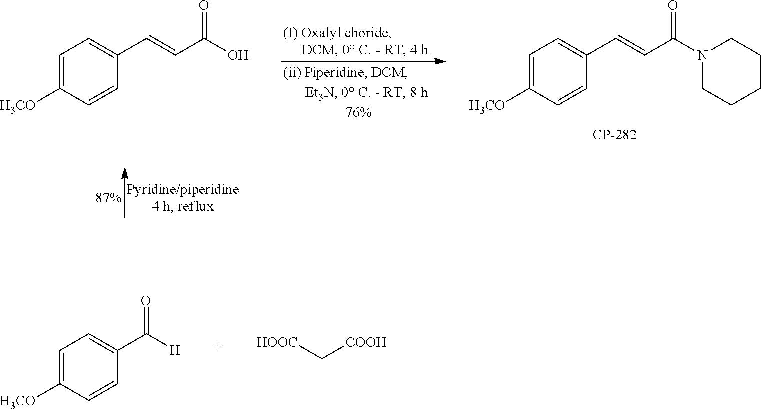 [SMILES: COc1ccc(/C=C/C(=O)O)cc1 ; COc1ccc(/C=C/C(=O)N2CCCCC2)cc1 ; COc1ccc(C=O)cc1 ; O=C(O)CC(=O)O]

To a solution of 4-methoxybenzaldhyde (1.0 g, 7.34 mmol) in pyridine (10 mL) at room temperature, malonic acid (1.67 g, 16.1 mmol) was added and stirred for 10 minutes, to this 0.5 ml of piperidine was added and then the reaction mixture was refluxed for 4 h. The progress of the reaction was monitored by TLC (EtOAc). After completion, the reaction mixture was quenched with 1M HCl (20 mL). The product precipitated as white crystalline solid. It was washed with dil. HCl (25 mL), diethyl ether (10 mL) and dried under vacuum to get (E)-4-methoxy cinnamic acid in 1.13 g (87% yield).

To a cold (0° C.) solution of 4-fluorocinnamic acid (1 g, 6.0 mmol) in DCM under N₂ atmosphere, oxalyl chloride (1.28 g, 10.1 mmol) was added slowly and the reaction mixture was slowly warmed to room temperature and stirred for 4 h. Excess oxalyl chloride was distilled off. Without further purification, the crude product was taken to the next step.

To a stirred solution of piperidine (0.46 g, 5.4 mmol) and Et₃N (0.73 g, 7.2 mmol) in DCM (20 mL) at 0° C., the above-synthesized acid chloride in DCM was added dropwise. After the addition, the reaction mass was slowly warmed to room temperature and stirred for 8 h. Progress of the reaction was monitored by TLC (hexane-EtOAc (60:40)). After completion, the DCM layer was washed with sat. NaHCO3 solution (100 mL), water (2×75 mL), dried over anhydrous Na₂SO₄ and concentrated under reduced pressure. The obtained crude product was column purified using hexane-EtOAc (90:10) to get the pure product as off-white solid in 1.01 g (yield 72%).

The compounds CP-286, CP-289, and CP-291 have been synthesized according to the procedure disclosed above for CP-282, whereas 4-methoxybenzaldhyde has been replaced by 4-nitrobenzaldehyde, 3,4-dimethoxybenzaldhyde, and 3-methoxy4-acetoxybenzaldhyde, respectively.

### Synthesis of (E)-cyclohexyl-1-(piperidin-1-yl)prop-2-en-1-one (CP-295)

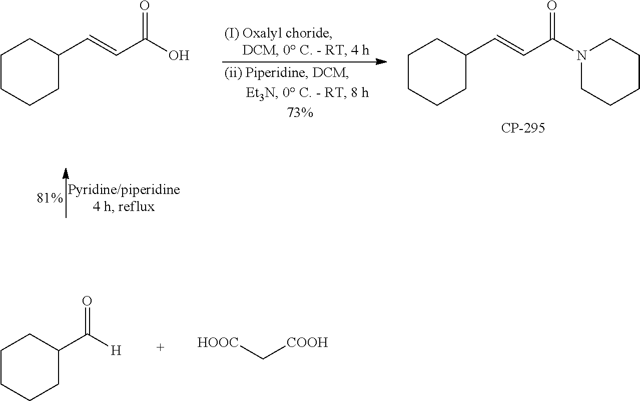 [SMILES: O=C(O)/C=C/C1CCCCC1 ; O=C(/C=C/C1CCCCC1)N1CCCCC1 ; O=CC1CCCCC1 ; O=C(O)CC(=O)O]

To a solution of cyclohexancarboxaldehyde (1.0 g, 8.91 mmol) in pyridine (10 mL) at room temperature, malonic acid (1.85 g, 17.8 mmol) was added and stirred for 10 minutes, to this 0.5 ml of piperidine was added and then the reaction mixture was refluxed for 4 h. The progress of the reaction was monitored by TLC (EtOAc). After completion, the reaction mixture was quenched with 1M HCl (20 mL). The product precipitated as white crystalline solid. It was washed with dil. HCl (25 mL), diethyl ether (10 mL) and dried under vacuum to get (E)-cyclohexylacrylic acid in 1.11 g (81% yield).

To a cold (0° C.) solution of cyclohexylacrylic acid (1 g, 6.4 mmol) in DCM under N₂ atmosphere, oxalyl chloride (1.23 g, 9.7 mmol) was added slowly and the reaction mixture was slowly warmed to room temperature and stirred for 4 h. Excess oxalyl chloride was distilled off. Without further purification, the crude product was taken to the next step.

To a stirred solution of piperidine (0.49 g, 5.7 mmol) and Et₃N (0.88 g, 8.6 mmol) in DCM (20 mL) at 0° C., the above-synthesized acid chloride in DCM was added dropwise. After the addition, the reaction mass was slowly warmed to room temperature and stirred for 8 h. Progress of the reaction was monitored by TLC (hexane-EtOAc (60:40)). After completion, the DCM layer was washed with sat. NaHCO₃ solution (100 mL), water (2×75 mL), dried over anhyd. Na₂SO₄ and concentrated under reduced pressure. The obtained crude product was column purified to get the pure product as off-white solid in 1.04 g (yield 73%).

### Synthesis of (E)-2-(nonenyl)-1-(piperidin-1-yl)prop-2-en-1-one (CP-296)

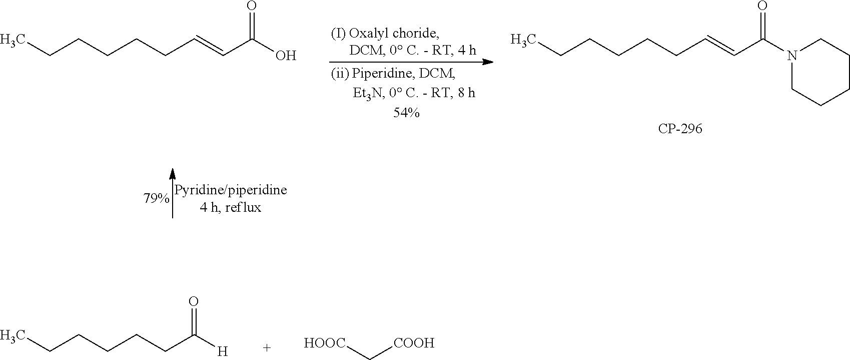

To a solution of heptanal (1.0 g, 8.75 mmol) in pyridine (10 ml) at room temperature, malonic acid (1.82 g, 17.5 mmol) was added and stirred for 10 minutes, to this 0.5 ml of piperidine was added and then the reaction mixture was refluxed for 4 h. The progress of the reaction was monitored by TLC (EtOAc). After completion, the reaction mixture was quenched with 1M HCl (20 mL). The product precipitated as white crystalline solid. It was washed with dil. HCl (25 mL), diethyl ether (10 mL) and dried under vacuum to get (E)-cyclohexylacrylic acid in 1.08 g (79% yield).

To a cold (0° C.) solution of 2-nonenoic acid (1 g, 6.4 mmol) in DCM under N₂ atmosphere, oxalyl chloride (1.21 g, 9.6 mmol) was added slowly and the reaction mixture was slowly warmed to room temperature and stirred for 4 h. Excess oxalyl chloride was distilled off. Without further purification, the crude product was taken to the next step.

To a stirred solution of piperidine (0.49 g, 5.7 mmol) and Et₃N (0.87 g, 8.5 mmol) in DCM (20 mL) at 0° C., the above-synthesized acid chloride in DCM was added dropwise. After the addition, the reaction mass was slowly warmed to room temperature and stirred for 8 h. Progress of the reaction was monitored by TLC (hexane-EtOAc (60:40)). After completion, the DCM layer was washed with sat. NaHCO₃ solution (100 mL), water (2×75 mL), dried over anhyd. Na₂SO₄ and concentrated under reduced pressure. The obtained crude product was column purified to get the pure product as a pale yellow liquid in 0.77 g (yield 54%).

### Synthesis of Diconjugated Coumaperine Derivatives

### Preparation of N-Crotonoylpiperidine

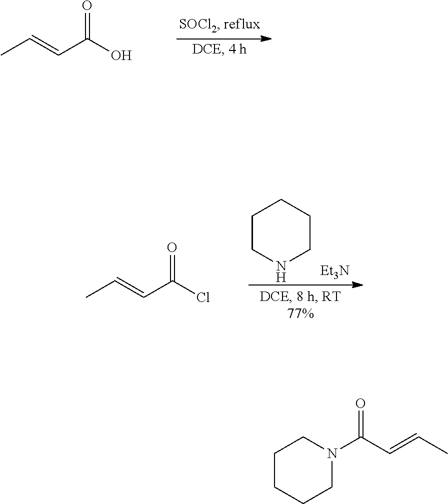

To a stirred solution of crotonic acid (50 g, 0.58 mol) in DCE (150 mL) at 0° C., SOCl₂ (58 mL, 0.669 mol) was added dropwise and refluxed for 4 h. After completion, excess SOCl₂ was removed under vacuum. Without further purification, the above-synthesized crotonyl chloride in DCE was added dropwise to a stirred solution of piperidine (58 mL, 0.58 mol) in DCE (50 mL) and Et₃N (55 g, 0.4175 mol) at 0° C. The reaction mass was slowly warmed to room temperature and stirred for 8 h. Progress of the reaction was monitored by TLC (hexane-EtOAc (60:40)). After completion, the DCE layer was washed with sat. NaHCO₃ solution (300 mL), water, dried over anhyd. Na₂SO₄ and concentrated under reduced pressure. A pale yellow viscous liquid was obtained in 68.5 g (yield 77%).

### Synthesis of 4-(tetrahydro-2H-pyran-2-yloxy)benzaldehyde

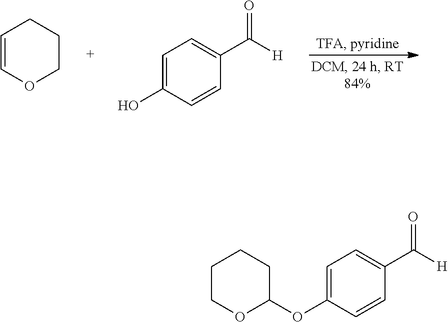 [SMILES: C1=COCCC1 ; O=Cc1ccc(O)cc1 ; O=Cc1ccc(OC2CCCCO2)cc1]

4-Tetrahydropyranyloxybenzaldehyde was prepared according to the literature reported procedure.

To a solution of 4-hydroxybenzaldehyde (5.80 g, 47.5 mmol) and 3,4-dihydro-2H -pyran (6.40 g, 76.1 mmol) in DCM (100 mL), trifluoroacetic acid (0.430 g, 2.50 mmol) was added dropwise and stirred for 24 h. The progress of the reaction was monitored by TLC. After completion, the crude reaction mixture was washed with water (3×50 mL), dried over Na₂SO₄ and concentrated under reduced pressure. The residue was washed with 10% NaOH (2×50 mL) solution and water to remove any unreacted starting material (4-hydroxybenzaldehyde). The final product was obtained as a dark brown oil in 8.23 g (yield 84%).

### Synthesis of (2E,4E)-5-(4-hydroxyphenyl)-1-(piperidin-1-yl)penta-2,4-dien-1-one (Coumaperine, CP)

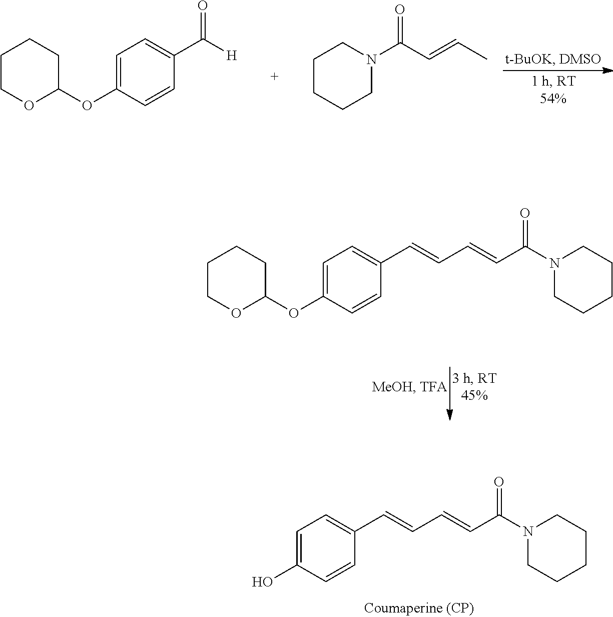 [SMILES: O=Cc1ccc(OC2CCCCO2)cc1 ; C/C=C/C(=O)N1CCCCC1 ; O=C(/C=C/C=C/c1ccc(OC2CCCCO2)cc1)N1CCCCC1 ; O=C(/C=C/C=C/c1ccc(O)cc1)N1CCCCC1]

To a stirred solution of N-crotonyl piperidine (0.37 g, 2.4 mmol) in DMSO (2 mL), 4-(tetrahydro-2H-pyran-2-yloxy)benzaldehyde (0.5 g, 2.4 mmol) was added and then potassium tert-butoxide (0.5 g) in 1.5 mL of DMSO was added slowly and stirred for 1 h. The reaction was monitored by TLC (chloroform-methanol (96:4)). After completion, the reaction mass was quenched with ice-cooled water (100 mL), during which off-white solid precipitated, it was filtered, washed with water, and dried under vacuum to get the product in 0.6 g (yield 54%).

### Deprotection

To a solution of tetrahydropyran protected coumaperine (0.6 g, 1.7 mmol) in methanol (8 mL), TFA (0.1 mL) was added dropwise and stirred at room temperature for 3 h. The reaction was monitored by TLC (chloroform-methanol ((96:4)). As the reaction progress, off-white solid precipitated. The reaction mass was cooled to 5° C., filtered and the off-white solid was washed with water (3×30 mL) and dried under vacuum. The obtained crude product was column purified (chloroform-methanol (95:5)) to get the pure product as off-white solid in 0.26 g (yield 45%). The overall yield of the reaction is 24%.

### Synthesis of (2E,4E)-5-phenyl-1-(piperidin-1-yl)penta-2,4-dien-1-one (CP-9)

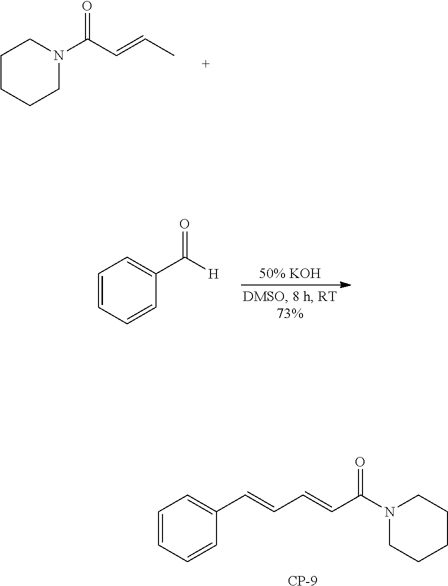 [SMILES: C/C=C/C(=O)N1CCCCC1 ; O=Cc1ccccc1 ; O=C(/C=C/C=C/c1ccccc1)N1CCCCC1 ; "CP-9"]

To a solution of N-crotonyl piperidine (1.2 g, 7.8 mmol) in DMSO (2 mL), benzaldehyde (0.68 g, 6.42 mmol) was added and then 50% aq. KOH (0.2 mL) solution was added dropwise and stirring continued for 8 h. The progress of the reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get pure product as pale-yellow solid in 1.4 g (yield 73%).

### Synthesis of (2E,4E)-5-(3,4-dimethoxyphenyl)-1-(piperidin-1-yl)penta-2,4-dien-1-one (CP-10)

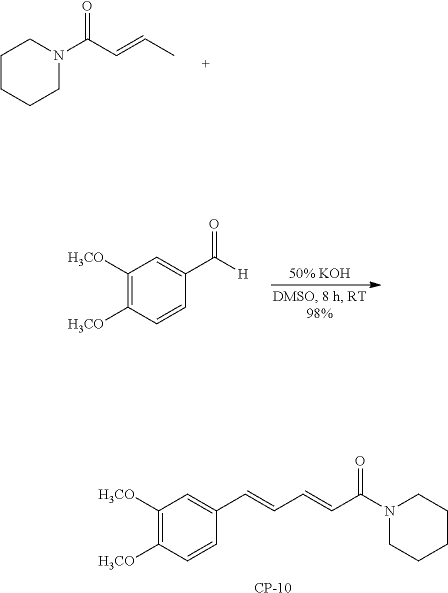 [SMILES: C/C=C/C(=O)N1CCCCC1 ; COc1ccc(C=O)cc1OC ; COc1ccc(/C=C/C=C/C(=O)N2CCCCC2)cc1OC ; "CP-10"]

To a solution of N-crotonyl piperidine (0.5 g, 3.27 mmol) in DMSO (2 mL), 3,4-dimethoxy benzaldehyde (0.55 g, 3.3 mmol) was added and then 50% aq. KOH (0.2 mL) solution was added dropwise and stirring continued for 8 h. The reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get a pure product as pale yellow solid in 0.98 g (yield 98%).

### Synthesis of (2E,4E)-1-(piperidin-1-yl)-5-(3,4,5-trimethoxyphenyl)penta-2,4-dien-1-one (CP-27)

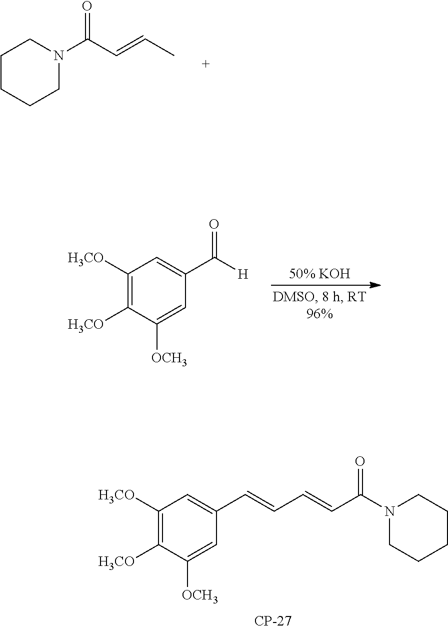 [SMILES: C/C=C/C(=O)N1CCCCC1 ; COc1cc(C=O)cc(OC)c1OC ; COc1cc(/C=C/C=C/C(=O)N2CCCCC2)cc(OC)c1OC ; "CP-27"]

To a solution of N-crotonyl piperidine (0.5 g, 3.27 mmol) in DMSO (2 mL), 3,4,5-trimethoxy benzaldehyde (0.64 g, 3.27 mmol) was added and then 50% aq. KOH (0.2 mL) solution was added dropwise and stirring continued for 8 h. The reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get the pure product as off-white solid in 1.04 g (yield 96%).

### Synthesis of (2E,4E)-5-(4-(dimethylamino)phenyl)-1-(piperidin-1-yl)penta-2,4-dien-1-one (CP-32)

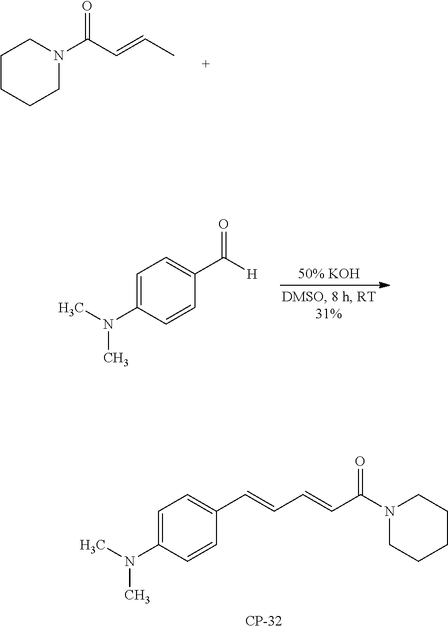 [SMILES: C/C=C/C(=O)N1CCCCC1 ; CN(C)c1ccc(C=O)cc1 ; CN(C)c1ccc(/C=C/C=C/C(=O)N2CCCCC2)cc1 ; "CP-32"]

To a solution of N-crotonyl piperidine (1 g, 6.54 mmol) in DMSO (2 mL), N,N-dimethyl benzaldehyde (1 g, 6.7 mmol) was added and then 50% aq. KOH (0.2 mL) solution was added dropwise and stirring continued for 8 h. The reaction was monitored by TLC (chloroform-methanol (96:4)). After completion, the reaction mixture was quenched with 5% aq. HCl (50 mL) and extracted with DCM (2×75 mL), the combined organic layer was washed with water (2×100 mL), dried over anhyd. Na₂SO₄ and concentrated under reduced pressure. The obtained crude product was recrystallized from isopropyl alcohol to get the pure product as pale-yellow solid in 0.58 g (yield 31%).

### Synthesis of (2E,4E)-5-(4-methoxyphenyl)-1-(piperidin-1-yl)penta-2,4-dien-1-one (CP-38)

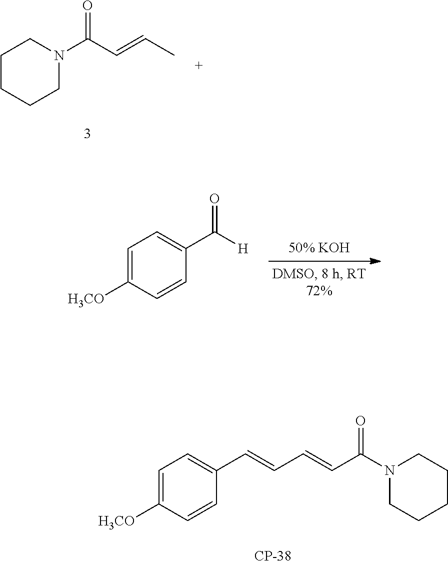 [SMILES: C/C=C/C(=O)N1CCCCC1 ; COc1ccc(C=O)cc1 ; COc1ccc(/C=C/C=C/C(=O)N2CCCCC2)cc1 ; "CP-38"]

To a solution of N-crotonyl piperidine (1.2 g, 7.8 mmol) in DMSO (2 mL), p-anisaldehyde (0.96 g 7.1 mmol) was added and then 50% aq. KOH (0.2 mL) solution was added dropwise and stirring continued for 8 h at room temperature. The progress of the reaction was monitored by TLC (chloroform-methanol (96:4)). After completion, the reaction mixture was quenched with aq. HCl (50 mL) and extracted with dichloromethane (2×75 mL), the combined organic layer was washed with water (2×100 mL), dried over anhyd. Na₂SO₄ and concentrated under reduced pressure. The obtained crude product was recrystallized from isopropyl alcohol to get the pure product as pale yellow solid in 1.36 g (yield 72%).

### Synthesis of (2E,4E)-1-(piperidin-1-yl)-5-(2,3,4-trimethoxyphenyl)penta-2,4-dien-1-one (CP-50)

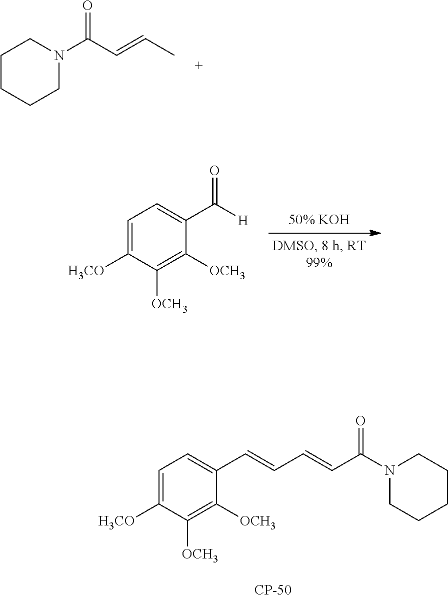 [SMILES: C/C=C/C(=O)N1CCCCC1 ; COc1ccc(C=O)c(OC)c1OC ; COc1ccc(/C=C/C=C/C(=O)N2CCCCC2)c(OC)c1OC ; "CP-50"]

To a solution of N-crotonyl piperidine (0.5 g, 3.27 mmol) in DMSO (2 mL), 2,3,4-trimethoxy benzaldehyde (0.64 g, 3.27 mmol) was added and then 50% aq. KOH (0.2 mL) solution was added dropwise and stirring continued for 8 h. The reaction was monitored by TLC (chloroform-methanol (96:4)). After completion, the reaction mixture was quenched with 5% aq. HCl (50 mL) and extracted with DCM (2×50 mL), the combined organic layer was washed with water (2×75 mL), dried over anhyd. Na₂SO₄ and concentrated under reduced pressure. The obtained crude product was recrystallized from isopropyl alcohol to get the pure product as pale-yellow solid in 1.07 g (yield 99%).

### Synthesis of (2E,4E)-5-(4-chlorophenyl)-1-(piperidin-1-yl)penta-2,4-dien-1-one (CP-123)

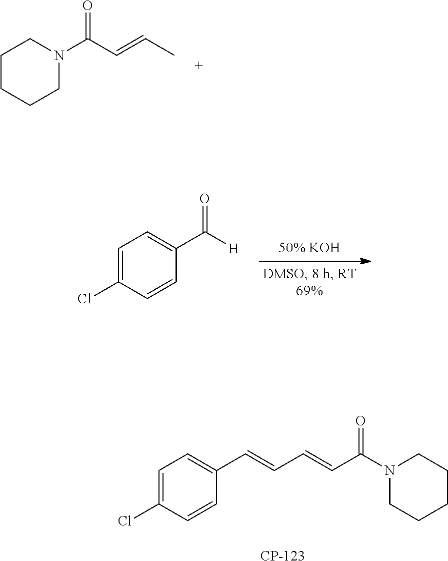 [SMILES: C/C=C/C(=O)N1CCCCC1 ; O=Cc1ccc(Cl)cc1 ; O=C(/C=C/C=C/c1ccc(Cl)cc1)N1CCCCC1 ; "CP-123"]

To a solution of N-crotonyl piperidine (1 g, 6.54 mmol) in DMSO (2 mL), 4-choloro benzaldehyde (0.94 g, 6.7 mmol) was added and then 50% aq. KOH (0.2 mL) solution was added dropwise and stirring continued for 8 h. The progress of the reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get the pure product as pale-yellow solid in 1.08 g (yield 69%).

### Synthesis of (2E,4E)-5-(4-(methylthio)phenyl)-1-(piperidin-1-yl)penta-2,4-dien-1-one (CP-147)

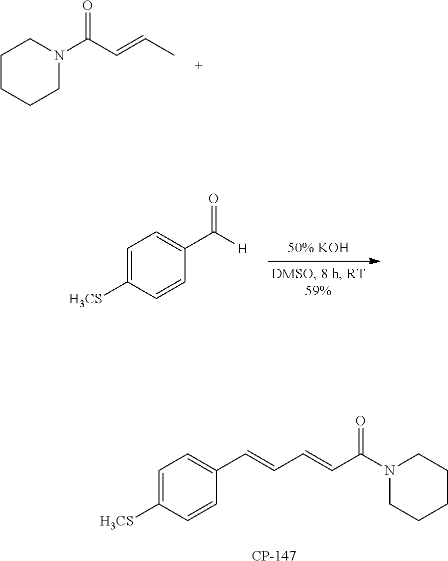

To a solution of N-crotonyl piperidine (1 g, 6.54 mmol) in DMSO (2 mL), 4-(methylthio)benzaldehyde (1.01 g, 6.7 mmol) was added and then 50% aq. KOH (0.2 mL) solution was added dropwise and stirring continued for 8 h. The reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get the pure product as yellow solid in 1.1 g (yield 59%).

### Synthesis of (2E,4E)-5-(4-fluorophenyl)-1-(piperidin-1-yl)penta-2,4-dien-1-one (CP-154)

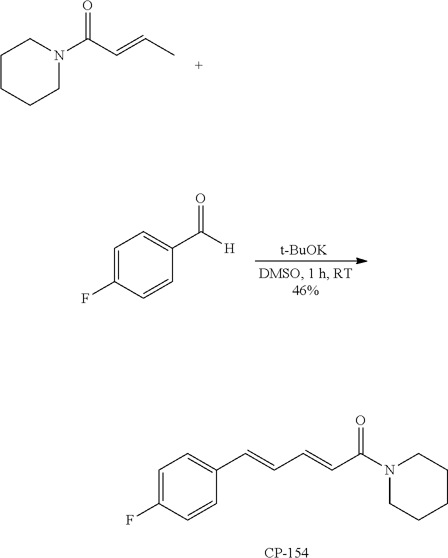 [SMILES: "t-BuOK" ; C/C=C/C(=O)N1CCCCC1 ; O=Cc1ccc(F)cc1 ; O=C(/C=C/C=C/c1ccc(F)cc1)N1CCCCC1 ; "CP-154"]

To a solution of N-crotonyl piperidine (0.5 g, 3.26 mmol) in DMSO (2 mL), 4-fluorobenzaldehyde (0.5 g 4.03 mmol) was added and then potassium tert-butoxide (0.5 g) in 1.5 mL of DMSO was added dropwise and stirred at room temperature for 1 h. The progress of the reaction was monitored by TLC (chloroform-methanol (96:4)). After completion, the reaction mass was quenched with ice-cooled water (50 mL) and extracted with EtOAc (2×50 mL), the combined organic layer was washed with 5% aq. HCl solution (50 mL), water (2×50 mL), dried over anhyd. Na₂SO₄ and concentrated under reduced pressure. The crude mass was column purified using hexane-EtOAc (90:10) to obtain the pure product as off-white solid in 0.39 g (46% yield).

### Synthesis of 4-((1E,3E)-5-oxo-5-(piperidin-1-yl)penta-1,3-dienyl)phenyl acetate (CP-158)

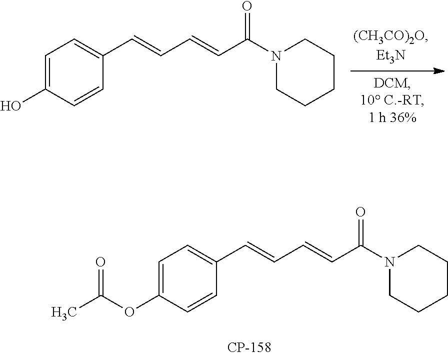 [SMILES: O=C(/C=C/C=C/c1ccc(O)cc1)N1CCCCC1 ; CC(=O)Oc1ccc(/C=C/C=C/C(=O)N2CCCCC2)cc1]

To a solution of coumaperine (0.257 g, 1 mmol) in DCM (10 mL), Et₃N (0.135 g, 1.3 mmol) was added and cooled to 10° C. To this acetic anhydride (0.136 g, 1.3 mmol) was added slowly and the reaction mass was brought to room temperature and stirred for 1 h. The progress of the reaction was monitored by TLC (chloroform-methanol (96:4)). After completion, the reaction mass was quenched with ice-cooled water (50 mL), extracted with DCM (2×50 mL), the combined organic layer was washed with 5% NaHCO₃ solution (50 mL), water (2×50 mL), dried over anhyd. Na₂SO₄ and concentrated under reduced pressure. The obtained crude product was recrystallized from hexane to get the pure product as off-white solid in 0.11 g (yield 36%).

### Synthesis of (2E,4E)-5-(4-(cyclopentyloxy)phenyl)-1-(piperidin-1-yl)penta-2,4-dien-1-one (CP-184)

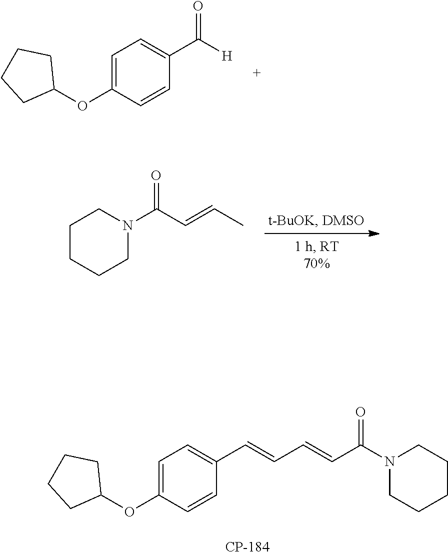 [SMILES: O=Cc1ccc(OC2CCCC2)cc1 ; C/C=C/C(=O)N1CCCCC1 ; O=C(/C=C/C=C/c1ccc(OC2CCCC2)cc1)N1CCCCC1]

To a solution of N-crotonyl piperidine (0.402 g, 2.6 mmol) in DMSO (2 mL), 4-(cyclopentyloxy)benzaldehyde (0.5 g 2.6 mmol) was added and then potassium tert-butoxide (0.5 g) in 1.5 mL of DMSO was added dropwise and stirred for 1 h. The progress of the reaction was monitored by TLC (chloroform-methanol (96:4)). After completion, the reaction mass was quenched with ice-cooled water (50 mL), off-white solid precipitated, it was filtered, washed with water (2×50 mL), and dried under vacuum to yield the pure product as off-white solid in 0.6 g (yield 70%).

### Synthesis of (2E,4E)-5-(3,5-dimethoxyphenyl)-1-(piperidin-1-yl)penta-2,4-dien-1-one (CP-193)

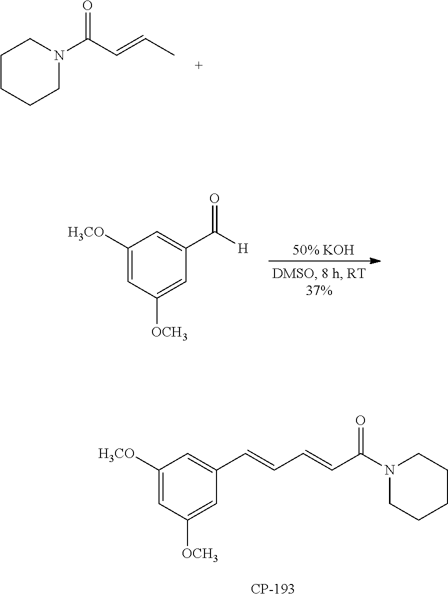 [SMILES: C/C=C/C(=O)N1CCCCC1 ; COc1cc(C=O)cc(OC)c1 ; "50% KOH" ; COc1cc(/C=C/C=C/C(=O)N2CCCCC2)cc(OC)c1 ; "CP-193"]

To a solution of N-crotonyl piperidine (0.5 g, 3.27 mmol) in DMSO (2 mL), 3,5-dimethoxybenzaldehyde (0.55 g, 3.3 mmol) was added and then 50% aq. KOH (0.2 mL) solution was added dropwise and stirring continued for 8 h. The reaction was monitored by TLC (chloroform-methanol (96:4). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get the pure product as pale yellow solid in 0.36 g (yield 37%).

### Synthesis of (2E,4E)-1-(piperidin-1-yl)-5-(2,4,6-trimethoxyphenyl)penta-2,4-dien-1-one (CP-194)

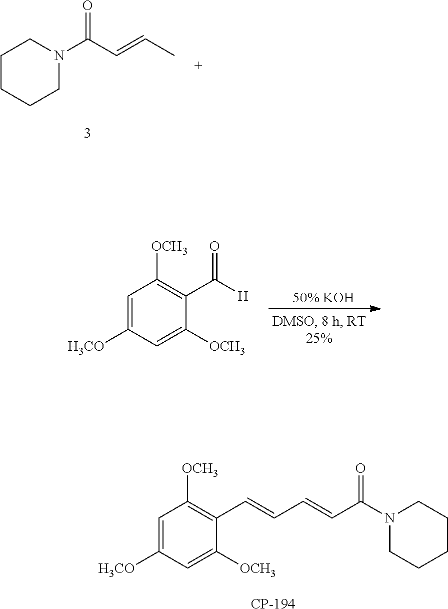

To a solution of N-crotonyl piperidine (0.5 g, 3.27 mmol) in DMSO (2 mL), 2,4,6-trimethoxybenzaldehyde (0.64 g, 3.27 mmol) was added and then 50% aq. KOH (0.2 mL) solution was added dropwise and stirring continued for 8 h. The reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The crude mass was column purified using hexane-EtOAc (90:10) to get the pure product as pale yellow solid in 0.270 g (yield 25%).

### Synthesis of (2E,4E)-5-(3,4-dihydroxyphenyl)-1-(piperidin-1-yl)penta-2,4-dien-1-one (CP-209)

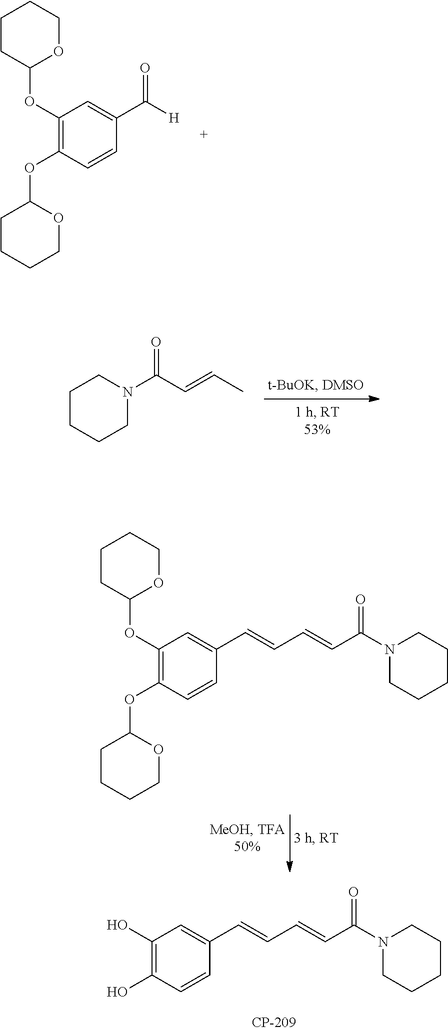 [SMILES: O=Cc1ccc(OC2CCCCO2)c(OC2CCCCO2)c1 ; C/C=C/C(=O)N1CCCCC1 ; O=C(/C=C/C=C/c1ccc(OC2CCCCO2)c(OC2CCCCO2)c1)N1CCCCC1 ; O=C(/C=C/C=C/c1ccc(O)c(O)c1)N1CCCCC1]

To a stirred solution of N-crotonyl piperidine (0.5 g, 3.26 mmol) in DMSO (2 mL), 3,4-bis(tetrahydro-2H-pyran-2-yloxy)benzaldehyde (1.1 g, 3.59 mmol) was added and then potassium tert-butoxide (0.54 g) in 1.5 mL of DMSO was added slowly and stirred for 1 h. The reaction was monitored by TLC (chloroform-methanol (96:4)). After completion, the reaction mass was quenched with ice-cooled water (50 mL), extracted with DCM (2×50 mL), washed with water (2×50 mL), dried over anhyd. Na₂SO₄ and concentrated under reduced pressure to yield the crude product as yellow solid in 0.763 g (yield 53%). Without further purification, it was taken to the next step.

### Deprotection

To a methanol solution (8 mL) of the above crude product (0.7 g, 1.58 mmol), TFA (0.1 mL) was added slowly and stirred at room temperature for 3 h. The reaction was monitored by TLC (chloroform-methanol ((96:4)). After completion, methanol was distilled off, the crude mixture was dissolved in EtOAc (50 mL), washed with 5% aq. HCl (50 mL) and then with 5% NaHCO₃ (50 mL), dried over anhyd. Na₂SO₄ and concentrated under reduced pressure. The obtained crude product was column purified (chloroform-methanol (90:10) to get the pure product as yellow solid in 0.21 g (yield 50%). Overall yield 26%.

### Deprotection of Piperine by BBr₃ Method

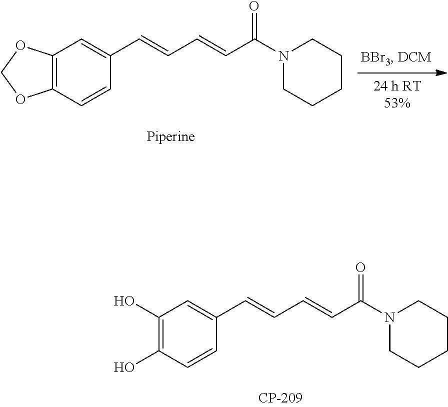 [SMILES: O=C(/C=C/C=C/c1ccc2c(c1)OCO2)N1CCCCC1 ; O=C(/C=C/C=C/c1ccc(O)c(O)c1)N1CCCCC1]

To a solution of piperine (500 mg, 1.75 mmol, 1 eq) in dichloromethane (DCM, 10 mL) at −15° C., a dichloromethane solution of BBr₃ (0.7 mL, 8.7 mmol, 5 equiv) was added at and slowly warmed to room temperature and stirring continued for 24 h. After the completion, DCM was removed under vacuum to yield yellow solid. It was then with water, DCM, and dried. The pure product was obtained by column purification, 254 mg (53%).

### Isolation of Piperine (PIP) from Black Pepper

Piperine was isolated from black pepper following the literature reported procedure

Black pepper (25 g) was powdered and extracted with hexane (2×75 mL) and the hexane layer was kept off. The residue was extracted with CHCl3 (3×75 mL). The combined chloroform layer was concentrated under reduced pressure. To the resulted residue, 5N ethanolic KOH solution was added and refluxed for 2 h. The ethanol layer was removed under reduced pressure, the resulted pasty mass was extracted with CHCl₃, the CHC₃ layer was washed with water (100 mL), 5% NaHCO₃ (50 mL), 5% HCl (50 mL), dried over Na₂SO₄ and concentrated under pressure to get the crude product. The crude product was dissolved in acetone and then hexane was slowly added, during which a white solid precipitated. It was filtered, washed with hexane (3×30 mL), and recrystallized from acetone to give to get the pure product as colorless crystals in 0.9 g.

Melting point (129-131° C.) and FT-IR spectra of the isolated piperine matched with literature reported value.

### Synthesis of (2E,4E)-1-(piperidin-1-yl)-5-(3,4,5-trihydroxyphenyl)penta-2,4-dien-1-one (CP-262-F1, CP-262-F2)

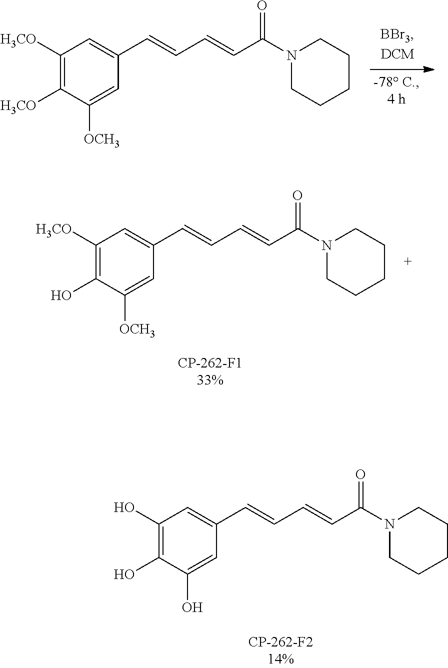 [SMILES: COc1cc(/C=C/C=C/C(=O)N2CCCCC2)cc(OC)c1OC ; COc1cc(/C=C/C=C/C(=O)N2CCCCC2)cc(OC)c1O ; O=C(/C=C/C=C/c1cc(O)c(O)c(O)c1)N1CCCCC1]

To a solution of CP-27 1 g (3.0 mmol) in DCM (30 mL) at −78° C., BBr₃ (26 mmol, 9 mL, 1M DCM solution) was added dropwise under N₂ atm. After the addition, the reaction mixture was slowly warmed to room temperature over 4 h. The progress of the reaction was monitored by TLC. After completion, the reaction mixture was cooled to −20° C. and quenched slowly with ice-cold water (caution: during quenching violent reaction occurs). The aqueous phase was extracted with EtOAc (2×50 mL), the combined organic layer was dried over Na₂SO₄ and removed under reduced pressure to get the crude product as dark brown solid. The pure product was obtained by column purification as off-white solid, CP-262-F1 in 0.31 g (33% yield) and as yellow solid, CP-262-F2 in 0.12 g (14% yield).

### Synthesis of 4-((1E,3E)-5-oxo-5-(piperidin-1-yl)penta-1,3-dienyl)phenyl mono and di acetate (CP-281-F1)

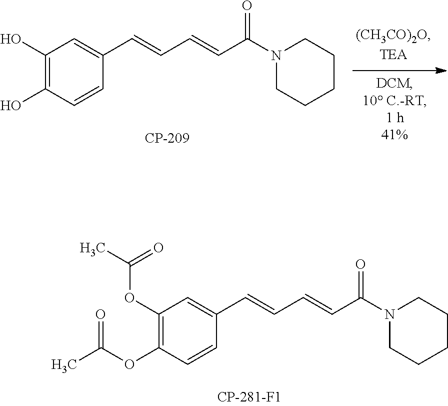 [SMILES: O=C(/C=C/C=C/c1ccc(O)c(O)c1)N1CCCCC1 ; CC(=O)Oc1ccc(/C=C/C=C/C(=O)N2CCCCC2)cc1OC(C)=O]

To a solution of CP-209 (0.250 g, 0.9 mmol) in DCM (10 mL), TEA (0.186 g, 1.8 mmol) was added and cooled to 10° C. To this acetic anhydride (0.186 g, 1.8 mmol) was added slowly and the reaction mass was brought to room temperature and stirred for 1 h. The progress of the reaction was monitored by TLC (chloroform-methanol (96:4)). After completion, the reaction mass was quenched with ice-cooled water (50 mL), extracted with DCM (2×50 mL), the combined organic layer was washed with 5% NaHCO₃ solution (50 mL), water (2×50 mL), dried over anhyd. Na₂SO₄ and concentrated under reduced pressure. The obtained crude product was column purified using hexane-EtOAc (80:20) to obtain the pure product as pale yellow solid (CP-281-F1) in 0.134 g (41% yield).

### Synthesis of Tetrahydro Piperine (CP-THP)

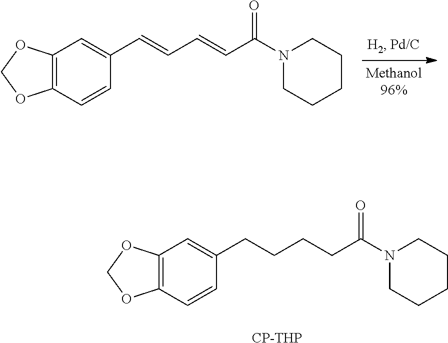 [SMILES: O=C(/C=C/C=C/c1ccc2c(c1)OCO2)N1CCCCC1 ; O=C(CCCCc1ccc2c(c1)OCO2)N1CCCCC1 ; "CP-THP"]

A mixture of methanolic solution of piperine (1 g) and 5% Pd/C (30 mg) was hydrogenated at 40 psi for 4 h at room temperature and the content was filtered, washed with methanol and concentrated under reduced pressure to give crude product. The crude product was column purified to get the pure product as white solid in 0.973 g (yield 96%).

### Synthesis of Triconjugated Coumaperine Derivatives

### Synthesis of N-Sorbyl Piperidine

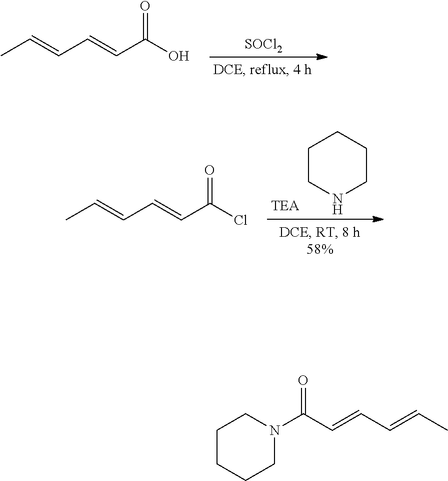

To a solution of sorbic acid (15 g, 0.1339 mol) in DCE (50 mL) at 0° C., thionyl chloride (14.5 mL, 0.20 mol) was added dropwise and then slowly warmed to room temperature and refluxed for 4 h. Excess thionyl chloride was removed under vacuum. Without further purification, the above-synthesized sorbyl chloride in DCE was added dropwise to a stirred solution of piperidine (13.2 mL, 0.1320 mol) and Et₃N (27 g, 0.2678 mol) in DCE (50 mL) at 0° C. After the addition, the reaction mass was slowly warmed to room temperature and stirred at that temperature for 8 h. The reaction was monitored by TLC (hexane-EtOAc (60:40). After completion, the reaction mass was washed with sat. NaHCO₃ solution, water, dried over anhyd. Na₂SO₄ and concentrated under reduced pressure. The crude mass was column purified (using hexane-EtOAc (60:40) as eluent) to get the pure product in 13.9 g (58% yield).

### Synthesis of (2E,4E,6E)-7-(4-methoxyphenyl)-1-(piperidin-1-yl)hepta-2,4,6-trien-1-one (CP-102)

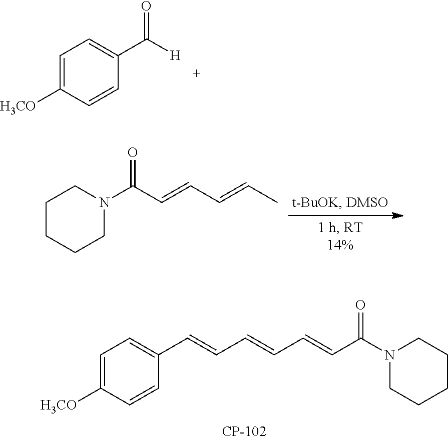 [SMILES: COc1ccc(C=O)cc1 ; C/C=C/C=C/C(=O)N1CCCCC1 ; COc1ccc(/C=C/C=C/C=C/C(=O)N2CCCCC2)cc1]

To a solution of N-sorbyl piperidine (0.5 g, 2.7 mmol)) in DMSO (2 mL), 4-methoxy benzaldehyde (0.4 g, 2.7 mmol) is added and then potassium tert-butoxide (0.375 g) in 1.5 mL of DMSO is added slowly and stirred for 1 h at room temperature. The reaction is monitored by TLC (chloroform-methanol (96:4)). After completion, the reaction mass is quenched with ice-cooled water and extracted with EtOAc, washed with 5% aq. HCl solution, water, dried over anhyd. Na2SO4 and concentrated under reduced pressure. The crude mass is column purified using hexane-EtOAc (90:10) to get the pure product as pale yellow solid in 0.13 g (yield 14%).

### Synthesis of (2E,4E,6E)-7-(benzo[d][1,3]dioxol-5-yl)-1-(piperidin-1-yl)hepta-2,4,6-trien-1-one (Pipertine, CP-155)

To a solution of N-sorbyl piperidine (0.5 g, 2.7 mmol) in DMSO (2 mL), benzo[d][1,3]dioxole-5-carbaldehyde (0.418 g, 2.7 mmol) was added and then potassium tert-butoxide (0.375 g) in 1.5 mL of DMSO was added slowly and stirred for 1 h at room temperature. The reaction progress was monitored by TLC (chloroform-methanol (96:4)). After completion, the reaction mass was quenched with ice-cooled water (50 mL) and extracted with EtOAc (2×50 mL), the combined organic layer was washed with 5% aq. HCl (2×30 mL), water (2×50 mL), dried over anhyd. Na₂SO₄ and concentrated under reduced pressure. The crude mass was column purified using hexane-EtOAc (95:5) to get the pure product as pale yellow solid in 0.12 g (yield 14%).

### Synthesis of (2E,4E,6E)-7-(3,4-dihydroxyphenyl)-1-(piperidin-1-yl)hepta-2,4,6-trien-1-one (CP-239)

To a stirred solution of N-sorbyl piperidine (0.5 g, 2.8 mmol) in DMSO (2 mL), 3,4-bis(tetrahydro-2H-pyran-2-yloxy)benzaldehyde (1.1 g, 3.5 mmol) was added and then potassium tert-butoxide (0.54 g) in 1.5 mL of DMSO was added slowly and stirred at room temperature for 1 h. The reaction was monitored by TLC (chloroform-methanol (96:4)). After completion, the reaction mass was quenched with ice-cooled water (50 mL), extracted with DCM (2×50 mL), the combined organic layer was washed with water (2×50 mL), dried over anhyd. Na2SO4 and concentrated under reduced pressure to get the bistetrahydropyran protected CP-239 in 0.96 g (yield 74%).

Deprotection: To a solution of bistetrahydropyran protected CP-239 (0.96 g, 2.0 mmol) in methanol (8 mL), TFA (0.1 mL) was added slowly and stirred at room temperature for 3 h. The reaction was monitored by TLC (chloroform-methanol ((96:4)). After completion, methanol was distilled off, the crude mixture was dissolved in EtOAc (50 mL), washed with 5% aq. HCl (50 mL) and then with 5% NaHCO3 (50 mL), dried over anhyd. Na₂SO₄ and concentrated under reduced pressure to get the crude product. The pure product was obtained after column purification (chloroform-methanol (90:10) as yellow solid in 0.22 g (yield 36%). Overall yield 26%.

### Synthesis of Monoconjugated Coumaperine Derivatives

The monoconjugated coumaperine derivatives depicted in FIG. 4 were synthesized from malonic acid and the corresponding aromatic aldehyde in two steps. Knoevenagel condensation of aldehyde and malonic acid in presence of base yields cinnamic acid derivatives. It was then converted to the corresponding acid chloride and subsequently reacted with piperidine to obtain monoconjugated coumaperine derivatives in good to excellent yields

The diconjugated derivatives shown in FIG. 5 were prepared by condensing the corresponding aldehyde with crotonic acid. A literature synthetic reported procedure was followed for the condensation. Initially, crotonic acid was converted to acid chloride by reacting with thionyl chloride and subsequently reacted with piperidine to obtain crotonyl piperidine. It was then condensed with the corresponding aromatic aldehydes in presence of base (KOH/t-BuOK) at room temperature to obtain the diconjugated coumaperine derivatives in low to excellent yields. The synthesized di coumaperine derivatives are depicted in FIG. 5.

CP-209, a dihydroxy coumaperine derivative was monoacetylated to obtain CP-281-F2. CP-209 was reacted with acetic anhydride in presence of trimethylamine at room temperature, yielded CP-281-F2 in 12% (FIG. 6). Similarly, CP-283 was prepared by acetylation of the corresponding trihydroxy derivative CP-262-F2, with acetic anhydride in excellent yield 81%.

### Synthesis of (E)-3-(benzoyl 4-acetoxy)-1-(piperidin-1-yl)prop-2-en-1-one (CP-284)

To a solution of 4-hydroxybenzaldhyde (1.0 g, 8.18 mmol) in pyridine (10 ml) at room temperature, malonic acid (1.7 g, 16.3 mmol) was added and stirred for 10 minutes, to this 0.5 ml of piperidine was added and then the reaction mixture was refluxed for 4 h. The progress of the reaction was monitored by TLC (EtOAc). After completion, the reaction mixture was quenched with 1M HCl (20 mL). The product precipitated as white crystalline solid. It was washed with dil. HCl (10 mL), diethyl ether (10 mL) and dried under vacuum to get (E)-4-hydroxycinnamic acid in 1.19 g (89% yield).

To a solution of (E)-4-hydroxycinnamic acid (1 g, 6.09 mmol) in DCM (20 mL), Et₃N (0.8 g, 7.9 mmol) was added and cooled to 10° C. To this acetic anhydride (0.8 g, 7.8 mmol) was added slowly and the reaction mass was brought to room temperature and stirred for 1 h. The progress of the reaction was monitored by TLC (chloroform-methanol (90:10)). After completion, the reaction mass was quenched with ice-cooled 5% aq. HCl (50 mL). The obtained white solid was filtered, washed with water, diethyl ether and dried under vacuum. The pure product obtained as white solid in 1.19 g (yield 95%). The melting point (209° C.-211° C.) of the product matched with literature reported value (205° C.-211° C.).

To a cold (0° C.) solution of 4-acetoxycinnamic acid (1 g, 4.8 mmol) in DCM under N₂ atmosphere, oxalyl chloride (0.92 g, 7.2 mmol) was added slowly and the reaction mixture was slowly warmed to room temperature and stirred for 4 h. Excess oxalyl chloride was distilled off. Without further purification the crude product was taken to next step.

To a stirred solution of piperidine (0.37 g, 4.4 mmol) and triethylamine (0.68 g, 6.6 mmol) in DCM (20 mL) at 0° C., the above synthesized acid chloride (1 g, 4.4 mmol) in DCM was added drop wise. After the addition, the reaction mass was slowly warmed to room temperature and stirred for 8 h. Progress of the reaction was monitored by TLC (hexane-EtOAc (60:40)). After completion, the DCM layer was washed with sat. NaHCO3 solution (100 mL), water (2×75 mL), dried over anhyd. Na2SO4 and concentrated under reduced pressure. The obtained crude product was column purified using hexane-EtOAc (90:10) to get the pure product as off-white solid in 0.82 g (yield 68%).

### Synthesis of (E)-3-(benzoyl 3,4-difluoro)-1-(piperidin-1-yl)prop-2-en-1-one (CP-287)

To a solution of 3,4-difluorobenzaldhyde (1.0 g, 7.03 mmol) in pyridine (10 ml) at room temperature, malonic acid (1.46 g, 14.0 mmol) was added and stirred for 10 minutes, to this 0.5 ml of piperidine was added and then the reaction mixture was refluxed for 4 h. The progress of the reaction was monitored by TLC (EtOAc). After completion, the reaction mixture was quenched with 1M HCl (20 mL). The product precipitated as white crystalline solid. It was washed with dil. HCl (10 mL), diethyl ether (10 mL) and dried under vacuum to get (E)-3,4-difluorocinnamic acid in 1.1 g (85% yield).

To a cold (0° C.) solution of 3,4-difluorociimainic acid (1 g, 5.4 mmol) in DCM under N₂ atmosphere, oxalyl chloride (1.0 g, 8.1 mmol) was added slowly and the reaction mixture was slowly warmed to room temperature and stirred for 4 h. Excess oxalyl chloride was distilled off. Without further purification the crude product was taken to next step.

To a stirred solution of piperidine (0.42 g, 4.9 mmol) and TEA (0.75 g, 7.4 mmol) in DCM (20 mL) at 0° C., the above synthesized acid chloride (1 g, 4.9 mmol) in DCM was added dropwise. After the addition, the reaction mass was slowly warmed to room temperature and stirred for 8 h. Progress of the reaction was monitored by TLC (hexane-EtOAc (60:40)). After completion, the DCM layer was washed with sat. NaHCO₃ solution (100 mL), water (2×75 mL), dried over anhyd. Na₂SO₄ and concentrated under reduced pressure. The obtained crude product was column purified using hexane-EtOAc (90:10) to get the pure product as off-white solid in 0.91 g (yield 74%).

### Synthesis of (E)-3-(benzoyl 4-trifluoromethy)-1-(piperidin-1-yl)prop-2-en-1-one (CP-288)

To a solution of 4-trifluoromethyl benzaldhyde (1.0 g, 5.74 mmol) in pyridine (10 ml) at room temperature, malonic acid (1.19 g, 11.4 mmol) was added and stirred for 10 minutes, to this 0.5 ml of piperidine was added and then the reaction mixture was refluxed for 4 h. The progress of the reaction was monitored by TLC (EtOAc). After completion, the reaction mixture was quenched with 1M HCl (20 mL). The product precipitated as white crystalline solid. It was washed with dil. HCl (10 mL), diethyl ether (10 mL) and dried under vacuum to get (E)-3,4-difluorocinnamic acid in 1.14 g (92% yield). The melting point (205 ° C.-207° C.) of the product matched with literature reported value (206° C.-208° C.).

To a cold (0° C.) solution of 4-trifluoromethyl cinnamic acid (1 g, 4.6 mmol) in DCM under N₂ atmosphere, oxalyl chloride (0.87 g, 6.9 mmol) was added slowly and the reaction mixture was slowly warmed to room temperature and stirred for 4 h. Excess oxalyl chloride was distilled off. Without further purification the crude product was taken to next step.

To a stirred solution of piperidine (0.36 g, 4.2 mmol) and TEA (0.65 g, 6.3 mmol) in DCM (20 mL) at 0° C., the above synthesized acid chloride (1 g, 4.2 mmol) in DCM was added dropwise. After the addition, the reaction mass was slowly warmed to room temperature and stirred for 8 h. Progress of the reaction was monitored by TLC (hexane-EtOAc (60:40)). After completion, the DCM layer was washed with sat. NaHCO₃ solution (100 mL), water (2×75 mL), dried over anhyd. Na2SO4 and concentrated under reduced pressure. The obtained crude product was column purified using hexane-EtOAc (90:10) to get the pure product as off-white solid in 0.85 g (yield 71%).

### Synthesis of (2E,4E)-5-(4-(cyclopentyloxy)-3-methoxyphenyl)-1-(piperidin-1-yl)penta -2,4-dien-1-one (CP-179)

To a solution of N-crotonyl piperidine (1 g, 6.5 mmol) in DMSO (2 mL), 4-(cyclopentyloxy)-3-methoxybenzaldehyde (1.58 g 7.1 mmol) was added and then potassium tert-butoxide (1.1 g) in 3 mL of DMSO was added dropwise and stirred for 1 h. Progress of the reaction was monitored by TLC (chloroform-methanol (96:4)). After completion, the reaction mass was quenched with ice cooled water (100 mL) and extracted with EtOAc (2×50 mL), the combined organic layer was washed with 5% aq. HCl solution (50 mL), water (2×50 mL), dried over anhyd. Na₂SO₄ and concentrated under reduced pressure. The crude mass was column purified using hexane-EtOAc (90:10) to get pure product as pale yellow oil in 0.278 g (yield 12%).

### Synthesis of (2E,4E)-5-napthyl-1-(piperidin-1-yl)penta-2,4-dien-1-one (CP-268)

To a solution of N-crotonyl piperidine (1.2 g, 7.8 mmol) in DMSO (2 mL), 2-napthaldehyde (0.98 g, 6.42 mmol) was added and then 50% aq. KOH (0.2 mL) solution was added dropwise and stirring continued for 8 h. Progress of the reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get pure product as pale yellow solid in 1.94 g (yield 85%).

### Synthesis of (2E,4E)-5-biphenyl-1-(piperidin-1-yl)penta-2,4-dien-1-one (CP-269)

To a solution of N-crotonyl piperidine (1.2 g, 7.8 mmol) in DMSO (2 mL), 1,1′-biphenyl]-4-carbaldehyde (1.28 g, 7.02 mmol) was added and then 50% aq. KOH (0.2 mL) solution was added dropwise and stirring continued for 8 h. Progress of the reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get pure product as pale yellow solid in 1.82 g (yield 82%).

### Synthesis of (2E,4E)-5-pyrene penta-2,4-dien-1-one (CP-272)

To a solution of N-crotonyl piperidine (1.2 g, 7.8 mmol) in DMSO (2 mL), 1-pyrene carboxaldehyde (1.80 g, 7.84 mmol) was added and then 50% aq. KOH (0.2 mL) solution was added dropwise and stirring continued for 8 h. Progress of the reaction was monitored by TLC (chloroform-methanol (96:4)). After completion, the reaction mixture was quenched with 5% aq. HCl (50 mL) and extracted with DCM (2×50 mL), the combined organic layer was washed with water (2×50 mL), dried over anhyd. Na₂SO₄ and concentrated under reduced pressure. The obtained crude product was recrystallized from isopropyl alcohol to get pure product as pale yellow solid in 2.29 g (yield 80%).

### Synthesis of 4-((1E,3E)-5-oxo-5-(piperidin-1-yl)penta-1,3-dienyl)phenyl acetate (CP-281-F2)

To a solution of CP-209 (0.250 g, 0.9 mmol) in DCM (10 mL), triethylamine (0.186 g, 1.8 mmol) was added and cooled to 10° C. To this acetic anhydride (0.186 g, 1.8 mmol) was added slowly and the reaction mass was brought to room temperature and stirred for 1 h. The progress of the reaction was monitored by TLC (chloroform-methanol (96:4)). After completion, the reaction mass was quenched with ice cooled water (50 mL), extracted with DCM (2×50 mL), the combined organic layer was washed with 5% NaHCO₃ solution (50 mL), water (2×50 mL), dried over anhyd. Na₂SO₄ and concentrated under reduced pressure. The obtained crude product was column purified using hexane-EtOAc (80:20) to obtain the pure product as pale yellow solid 0.035 g (12% yield).

### Synthesis of 4-((1E,3E)-5-oxo-5-(piperidin-1-yl)penta-1,3-dienyl)phenyl triacetate (CP-283)

To a solution of CP-262-F2 (0.100 g, 0.34 mmol) in DCM (10 mL), triethylamine (0.105 g, 1.0 mmol) was added and cooled to 10° C. To this acetic anhydride (0.105 g, 1.0 mmol) was added slowly and the reaction mass was brought to room temperature and stirred for 1 h. The progress of the reaction was monitored by TLC (chloroform-methanol (96:4)). After completion, the reaction mass was quenched with ice cooled water (50 mL), extracted with DCM (2×50 mL), the combined organic layer was washed with 5% NaHCO₃ solution (50 mL), water (2×50 mL), dried over anhyd. Na₂SO₄ and concentrated under reduced pressure. The obtained crude product was column purified using hexane-EtOAc (80:20) to obtain the pure product as off-white solid in 0.116 g (81% yield).

### Piperlongumine Isolation

Piperlogumine was isolated according to the literature reported procedure.

### Synthesis of Monohydroxypiperlongumine (PL-07)

To a suspension of AlCl₃ (1.05 g, 7.8 mmol) in DCM (15.0 mL) at room temperature, a solution of piperlongumine (0.25 g, 0.78 mmol) in DCM (5.0 mL) was added dropwise under N₂ atm and stirred for 1 h at room temperature. The progress of the reaction was monitored by TLC. After completion, the reaction mixture was cooled to 0° C., quenched with 1% HCl solution (50 mL) and poured into sat. brine solution (50 mL), extracted with EtOAc (2×100 mL), the combined organic layer was washed with sat. brine solution (50 mL), dried over Na₂SO₄ and concentrated under reduced pressure to get crude product. Pure product was obtained after column purification as yellow solid in 0.19 g (yield 82%).

### Synthesis of Di and Trihydroxypiperlongumine (PL-18 and PL-25)

To a suspension of AlCl₃ (3.15 g, 23.6 mmol) in DCM (30.0 mL) at room temperature, a solution of piperlongumine (0.25 g, 0.78 mmol) in DCM (5.0 mL) was added dropwise under N₂ atm and stirred for 24 h at room temperature. The progress of the reaction was monitored by TLC. After completion, the reaction mixture was cooled to 0° C., quenched with 1% HCl solution (50 mL) and poured into sat. brine solution (50 mL), extracted with EtOAc (2×100 mL), the combined organic layer was washed with sat. brine solution (50 mL), dried over Na₂SO₄ and concentrated under reduced pressure to get crude product. Pure product was obtained after column purification. DHPL obtained as off-white solid 123 mg (yield 54%). THPL as yellow solid 69.3 mg (yield 32%).

### Synthesis of PL-16A, PL-31 (SPL), PL-23B

Knoevenagel condensation of 3,4,5-trimethoxybenzaldehyde: to a solution of 3,4,5-trimethoxy benzaldehyde (1.0 g, 5.09 mmol) in pyridine (40 ml) at room temperature, malonic acid (1.07 g, 10.19 mmol) was added and stirred for 10 minutes, to this 0.5 ml of piperidine was added and the reaction mixture was refluxed for 4 h. Progress of the reaction was monitored by TLC. After completion, the reaction mixture was quenched with 1M HCl (20 mL). The product precipitated as white crystalline solid. It was washed with dil. HCl (50 mL), diethyl ether (50 mL) and dried under vacuum to get 3,4,5-trimethoxycinnamic acid in 1.09 g (89% yield). The melting point (127° C.-128° C.) of the product matched with literature reported value (129° C.).

To a solution of 3,4,5-trimethoxycinnamic acid (500 mg, 2.09 mmol) i DCM at 0° C., under N₂ atmosphere, oxalyl chloride (532.8 mg, 4.19 mmol) was added slowly and the reaction mixture was slowly warmed to room temperature and stirred for 4 h. Excess oxalyl chloride was distilled off. Without further purification the crude product was taken to next step.

To a solution of 2-piperidone (212.4 mg, 2.14 mmol) in toluene (20 mL) at room temperature, the above synthesized acid chloride (500 mg, 1.94 mmol) in 5 ml of toluene was added dropwise. After the addition, the reaction mass was slowly heated to reflux and stirred for 16 h. Progress of the reaction was monitored by TLC (hexane-EtOAc (60:40)). After completion, the reaction mass was cooled to room temperature, 20 ml of EtOAc was added and the organic layer was washed with sat. NaHCO₃ solution (2×50 mL), water (2×75 mL), dried over anhyd. Na₂SO₄ and concentrated under reduced pressure. The obtained crude product was column purified to get the pure products, PL-16A 67 mg (yield 7%), PL-31 520 mg (yield 83%) and PL-23B 42 mg (yield 6%).

### General Procedure for Synthesis of Piperlongumine Derivatives

To the saturated piperlongumine (SPL) (1 equiv.), amine (5 equiv.) was added and stirred at room temperature. Progress of the reaction was monitored by TLC (hexane-ethyl acetate (1:1). Starting material consumed within 30-45 min with all the amines except glycine and serine. After completion, the reaction mixture was quenched with 5% aq. HCl (15 mL) and extracted with ethyl acetate (2×15 mL), the combined organic layer was washed with water (1×15 mL), 5% NaHCO₃ solution (15 mL), water (2×15 mL), dried over anhyd. Na₂SO₄ and concentrated under reduced pressure. To the crude reaction mass, 2 mL of diethyl ether was added, and the reaction mass was filtered and dried to get pure product 60-76% yield. Various piperlongumine derivatives have been synthesized based on the general procedure by varying the corresponding amine, as depicted in the synthetic schemes below.

### Synthesis of (E)-N-ethyl-3-(3,4,5-trimethoxyphenyl)acrylamide (PL-17)

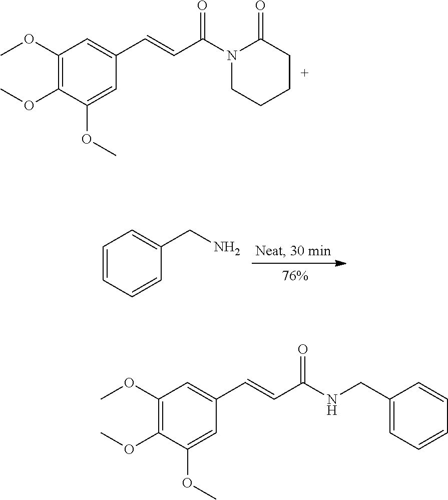 [SMILES: COc1cc(/C=C/C(=O)N2CCCCC2=O)cc(OC)c1OC ; NCc1ccccc1 ; COc1cc(/C=C/C(=O)NCc2ccccc2)cc(OC)c1OC]

### Synthesis of (E)-N-benzyl-3-(3,4,5-trimethoxyphenyl)acrylamide (PL-20)

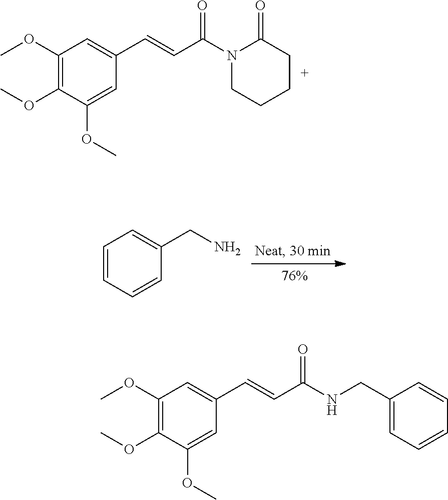 [SMILES: COc1cc(/C=C/C(=O)N2CCCCC2=O)cc(OC)c1OC ; NCc1ccccc1 ; COc1cc(/C=C/C(=O)NCc2ccccc2)cc(OC)c1OC]

### Synthesis of (E)-N-(2-hydroxyethyl)-3-(3,4,5-trimethoxyphenyl)acrylamide (AE-02)

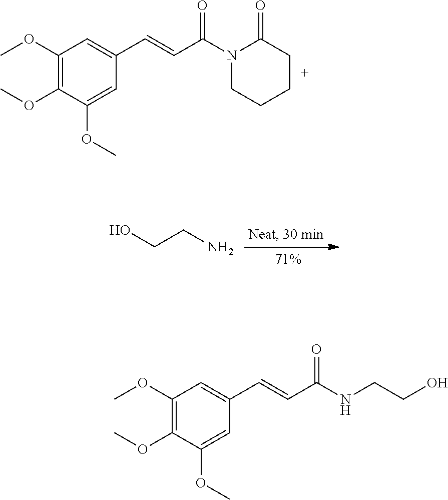

### Synthesis of (E)-N-allyl-3-(3,4,5-trimethoxyphenyl)acrylamide (AE-04)

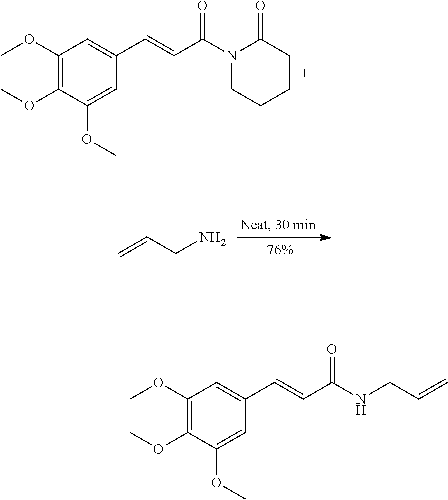

### Synthesis of (E)-N-ethyl-3-(4-hydroxy-3,5-dimethoxyphenyl)acrylamide (AE-10)

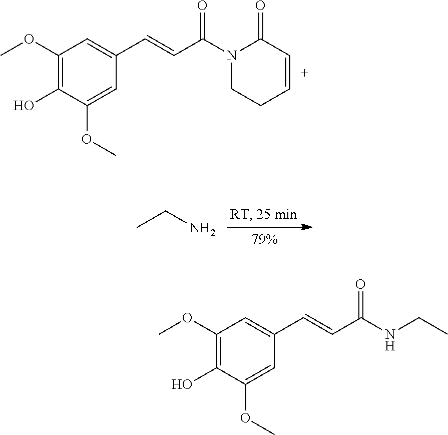

### Synthesis of (E)-N-(4-hydroxyphenethyl)-3-(3,4,5-trimethoxyphenyl)acrylamide (AE-11)

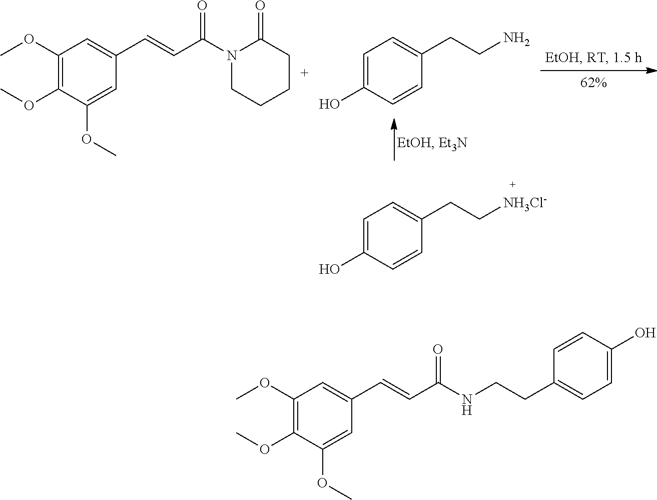

### Synthesis of (E)-N-(3,4-dihydroxyphenethyl)-3-(3,4,5-trimethoxyphenyl)acrylamide (AE-17)

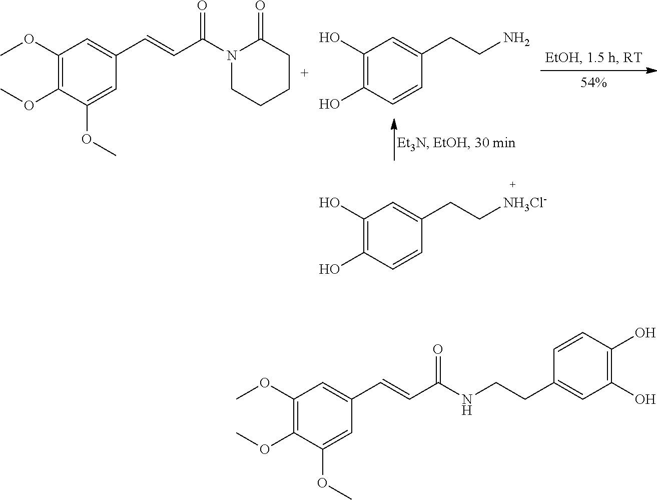

### Synthesis of (E)-1-(piperidin-1-yl)-3-(3,4,5-trimethoxyphenyl)prop-2-en-1-one (AE-45)

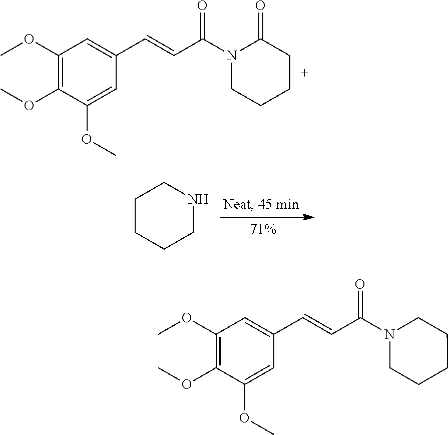 [SMILES: COc1cc(/C=C/C(=O)N2CCCCC2=O)cc(OC)c1OC ; C1CCNCC1 ; COc1cc(/C=C/C(=O)N2CCCCC2)cc(OC)c1OC]

### Synthesis of (E)-N-isopropyl-3-(3,4,5-trimethoxyphenyl)acrylamide (AE-68)

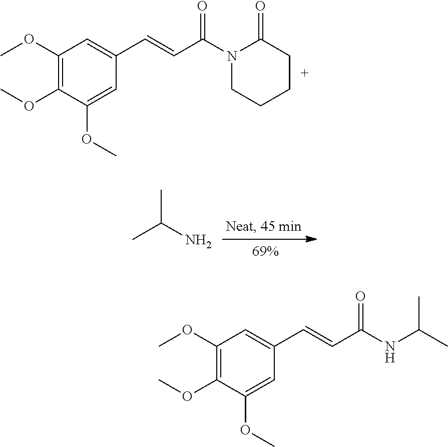

### Synthesis of (E)-ethyl 2-(3-(3,4,5-trimethoxyphenyl)acrylamido)acetate (AE-73)

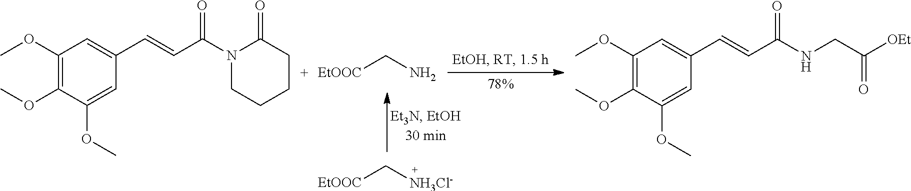

To a stirred solution of glycine ethyl ester. HCl (66.04 mg, 0.473 mmol) in ethanol (0.8 mL), triethylamine 48.2 mg, 0.473 mmol) was added and stirred for 30 min. To this SPL (50.0 mg, 0.157 mmol) was added and stirred at room temperature. The reaction was monitored by TLC (Hexane-ethyl acetate (1:1). Starting material consumed within 1.5 h. After completion, the reaction mixture was quenched with 5% aq. HCl (10 mL) and extracted with ethyl acetate (2×20 mL), the combined organic layer was washed with water (1×20 mL), 5% NaHCO₃ solution (20 mL), water (2×20 mL), dried over anhyd. Na₂SO₄ and concentrated under reduced pressure. To the crude reaction mass, 2 mL of diethyl ether was added, and the reaction mass was filtered and dried to get pure product as white solid in 40 mg (yield 78%).

### Synthesis of (E)-2-(3-(3,4,5-trimethoxyphenyl)acrylamido)acetic acid (AE-77)

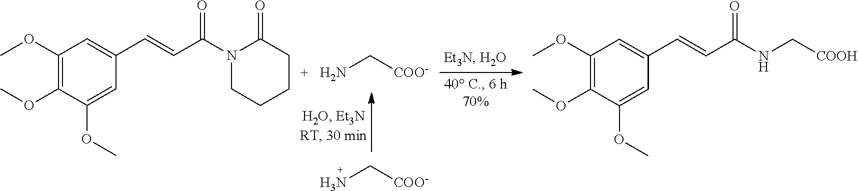

To stirred solution of glycine (70.5 mg, 0.94 mmol) in water (44 μl), trimethylamine (320 mg) was added and stirred at room temperature for 30 min, to this SPL (100.0 mg, 0.313 mmol) was added and stirred at 40° C. Progress of the reaction was monitored by TLC (chloroform-methanol (1:1). Starting material consumed within 6 h. After completion, the reaction mixture was quenched with 5% aq. HCl (20 mL) and extracted with ethyl acetate (3×20 mL), the combined organic layer was washed with water (1×20 mL), dried over anhyd. Na₂SO₄ and concentrated under reduced pressure. To the crude reaction mass, 3 mL of diethyl ether was added, and the reaction mass was filtered and dried to get pure product as white solid 63 mg (yield 70%).

### Synthesis of Diacetylated Curcumin (AC-CU)

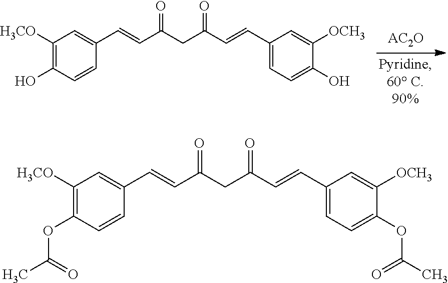 [SMILES: COc1cc(/C=C/C(=O)CC(=O)/C=C/c2ccc(O)c(OC)c2)ccc1O ; COc1cc(/C=C/C(=O)CC(=O)/C=C/c2ccc(OC(C)=O)c(OC)c2)ccc1OC(C)=O]

To a solution of curcumin (100 mg, 0.271 mmol) in pyridine (2 mL), acetic anhydride (0.82 g, 0.81 mmol) was added slowly at room temperature and stirred for 1 h and slowly heated to 60° C. for 3 h. The progress of the reaction was monitored by TLC (chloroform-methanol (90:10). After completion, the reaction mass was quenched with ice-cooled 5% aq. HCl (50 mL extracted with ethyl acetate (2×50 mL), the combined organic layer was washed with 5% NaHCO₃ solution (50 mL), water (2×50 mL), dried over anhyd. Na₂SO₄ and concentrated under reduced pressure. The obtained crude product was column purified using hexane-EtOAc (70:30) to obtain the pure product as pale semi solid 110 mg (90% yield). The formation of acetylated curcumin (AC-CU) is confirmed by melting point comparison with literature reported value. The melting point (155° C.-156° C.) of as synthesized product AC-CU matched with literature reported value, 155° C.-157° C.

### Synthesis of Tetrahydrocurcumin (HCU)

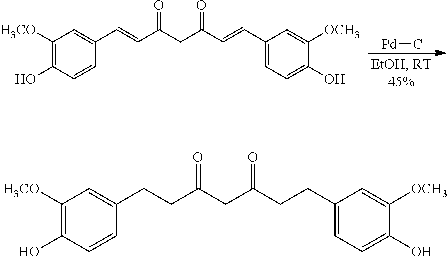

To a solution of curcumin (200 mg, 0.53 mmol) in ethanol (15 mL) at room temperature, 5% Pd-C (100 mg) was added, after degassing, the mixture was hydrogenated at room temperature and at atmospheric pressure for 6 hours. The mixture was filtered through a column of celite and the solvent was evaporated. The residue was purified by column chromatography to obtain tetrahydrocurcumin as off-white solid in 91 mg (yield 45%).

### Synthesis of Diacetylated Tetrahydrocurcumin (AC-THC)

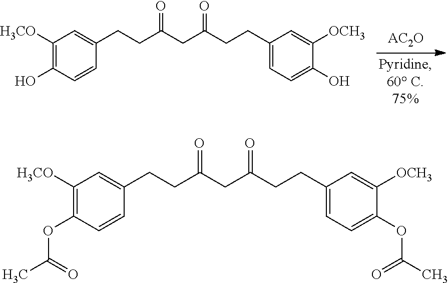 [SMILES: COc1cc(CCC(=O)CC(=O)CCc2ccc(O)c(OC)c2)ccc1O ; COc1cc(CCC(=O)CC(=O)CCc2ccc(OC(C)=O)c(OC)c2)ccc1OC(C)=O]

To a solution of HCU (100 mg, 0.268 mmol) in pyridine (2 mL), acetic anhydride (0.82 g, 0.80 mmol) was added slowly at room temperature and stirred for 1 h and slowly heated to 60° C. for 4 h. The progress of the reaction was monitored by TLC (chloroform-methanol (90:10). After completion, the reaction mass was quenched with ice-cooled 5% aq. HCl (50 mL extracted with DCM (2×50 mL), the combined organic layer was washed with 5% NaHCO₃ solution (50 mL), water (2×50 mL), dried over anhyd. Na₂SO₄ and concentrated under reduced pressure. The obtained crude product was column purified using hexane-EtOAc (80:20) to obtain the pure product as pale semi solid 92 mg (75% yield).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A compound represented by Formula I: 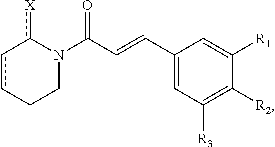
wherein: [symbol] represents a single or a double bond; X comprises H, S, O, NH or NH₂; each of R₁, R₂ and R₃ independently represents hydrogen, a substituent is selected from the group comprising halogen, nitro, —OR, —NR₂, —NHR, —SR, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; wherein each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; if R₁ and R₃ are hydrogen, then R₂ is devoid of halo, —OR, and nitro; or if R₁, R₃ or both are —OR, then R₂ is devoid of —OR; and wherein at least one of R₁, R₂ and R₃ represents said substituent or is

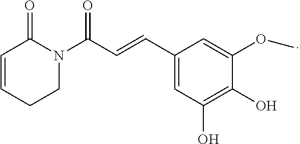 [SMILES: COc1cc(/C=C/C(=O)N2CCC=CC2=O)cc(O)c1O]

2. The compound of claim 1, wherein said compound is selected from the group consisting of:

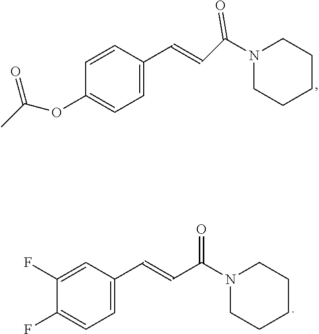 [SMILES: CC(=O)Oc1ccc(/C=C/C(=O)N2CCCCC2)cc1 ; O=C(/C=C/c1ccc(F)c(F)c1)N1CCCCC1]

3.-10. (canceled)

11. A pharmaceutical composition comprising the compound of claim 1, and an acceptable carrier.

12.-19. (canceled)

20. A method for treating a subject afflicted with a disease selected from the group consisting of: an inflammatory disease, an infectious disease, an amyloid aggregates-related disease, and cell proliferation related disease, comprising: administering to said subject a therapeutically effective amount of compound represented by (i) Formula I: 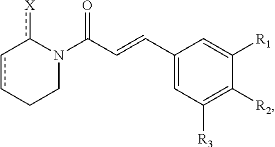
wherein: [symbol] represents a single or a double bond; X comprises H, S, O, NH or NH₂; each of R₁, R₂ and R₃ independently represents hydrogen, a substituent is selected from the group comprising halogen, nitro, —OR, —NR₂, —NHR, —SR, —OCOR, —CO₂R, —NCOR, —CONR₂, —SO₂R, —CN, optionally substituted C₁-C₆ alkyl, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, and optionally substituted bicyclic cycloalkyl or any combination thereof; wherein each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; if R₁ and R₃ are hydrogen, then R₂ is devoid of halo, —OR, and nitro; or if R₁ R₃ or both are —OR, then R₂ is devoid of —OR, and wherein at least one of R₁, R₂ and R₃ represents said substituent wherein each of R₁, R₂ and R₃ independently represent hydrogen or a substituent selected from the group comprising —OR, —NR₂, —NHR, —SR, —OCOR, —CO₂R, —NCOR, —CONR₂, wherein at least two of R₁, R₂ and R₃ represent the substituent, and wherein R is as described hereinabove; or (ii) by Formula IB: 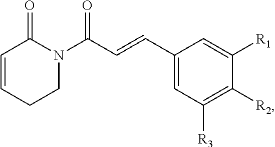
wherein each of R₁, R₂ and R₃ independently represent hydrogen or a substituent selected from the group comprising —OR, —NR₂, —NHR, —SR, —OCOR, —CO₂R, —NCOR, —CONR₂, wherein at least two of R₁, R₂ and R₃ represent the substituent, and wherein each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, and optionally substituted aryl or a combination thereof; thereby treating a subject afflicted with the disease selected from the group consisting of: an inflammatory disease, an infectious disease, and an amyloid aggregates-related disease.

21. The method of claim 20, wherein said infectious disease comprises a load of a microorganism, a biofilm derived therefrom, or both.

22. The method of claim 21, wherein said microorganism is a bacterium or a fungus.

23. The method of claim 22, wherein said bacterium belongs to a genus selected form the group consisting of: Vibrio, Salmonella, Staphylococcus, Pseudomonas, Chromobacterium Agrobacterium, Bacillus, Acinetobacter, Streptococcus, Salmonella, Erwinia, and Escherichia.

24. The method of claim 20, wherein said pharmaceutical composition is characterized by having a median lethal concentration (LC50) of 1-500 μM.

25. The method of claim 20, wherein said subject is afflicted with a disease selected from the group consisting of: Chron's disease, Ulcerative colitis, an auto-immune disease, Hodgkin lymphoma, cystic fibrosis, psoriasis, pneumonia, a blood stream infection, an infectious wound, an infectious burn wound, a urinary tract infection, a medical instrument related septic or aseptic complication, or any combination thereof.
